(19) United States
(12) Patent Application Publication
(10) Pub. No.: US 2024/0115718 A1
(43) Pub. Date: Apr. 11, 2024

(54) METHODS OF USING ANTI-CD79B IMMUNOCONJUGATES TO TREAT DIFFUSE LARGE B-CELL LYMPHOMA

(71) Applicant: Genentech, Inc., South San Francisco, CA (US)

(72) Inventors: Jamie Harue HIRATA, San Carlos, CA (US); Lisa Linnea MUSICK, Kentfield, CA (US)

(73) Assignee: Genentech, Inc., South San Francisco, CA (US)

(21) Appl. No.: 18/505,929

(22) Filed: Nov. 9, 2023

Related U.S. Application Data
(63) Continuation of application No. PCT/US2022/072267, filed on May 11, 2022.
(60) Provisional application No. 63/187,858, filed on May 12, 2021.

(51) Int. Cl. A61K 47/68; A61K 31/4412; A61P 35/00; C07K 16/28

(52) U.S. Cl. CPC A61K 47/6803; A61K 31/4412; A61K 47/6849; A61P 35/00; C07K 16/2803; C07K 16/2887; A61K 2039/505

(57) ABSTRACT

Provided herein are methods of treating B-cell proliferative disorders (such as diffuse large B-cell lymphoma “DLBCL”) using immunoconjugates comprising anti-CD79b antibodies in combination with an immunomodulatory agent (such as lenalidomide) and an anti-CD20 antibody (such as obinutuzumab or rituximab).

### CROSS-REFERENCE TO RELATED APPLICATIONS

This application claims the benefit of U.S. Provisional Application No. 63/187,858, filed May 12, 2021, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392054040SEQLIST.TXT, date recorded: May 4, 2021, size: 64 KB).

### FIELD OF THE INVENTION

The present disclosure relates to methods of treating B-cell proliferative disorders, e.g., diffuse large B cell lymphoma (DLBCL) by administering an immunoconjugate comprising an anti-CD79b antibody in combination with an immunomodulatory agent (e.g., lenalidomide) and an anti-CD20 antibody (e.g., obinutuzumab or rituximab).

### BACKGROUND OF THE INVENTION

Non-Hodgkin lymphoma (NHL) is the most common hematologic malignancy in the world and the thirteenth most common cancer overall (Bray et al., (2018) CA Cancer J Clin, 68:394-424). Diffuse large B-cell lymphoma (DLBCL) is an aggressive subtype of NHL, accounting for approximately 32.5% of all NHL cases. DLBCL originates from mature B-cells and has a median survival of <1 year in untreated patients (Rovira et al., (2015) Ann Hematol, 378:1396-1407). A majority of DLBCL cells express CD20, a membrane antigen that is important in cell cycle initiation and differentiation (Anderson et al., (1984) Blood, 63:1424-1433).

First-line treatment of DLBCL has consisted of an anti-CD20 monoclonal antibody treatment in combination with a multi-agent chemotherapy (National Comprehensive Cancer Network 2018; Shen et al., (2018) Lancet vol 5, e264). For patients who are not cured by first-line therapy, high-dose chemotherapy followed by autologous stem cell transplantation offers a second chance for long-term remission. For relapsed/refractory (R/R) DLBCL patients who are not eligible for stem cell transplantation due to age, comorbidities, or other factors, there are different treatment options, including various chemoimmunotherapies. These chemoimmunotherapies, however, tend to be used with the goal of palliation rather than long-term survival. Recently approved treatments for the R/R DLBCL setting include CAR-T therapies and polatuzumab vedotin-piiq in combination with bendamustine and rituximab.

Approximately half of patients with relapsed DLBCL fail to respond to second-line therapy because of refractory disease (Gisselbrecht et al., (2010) J Clin Oncol, 28:4184-4190). Patients who either relapse after or are ineligible for stem cell transplantation because of refractory disease or frailty have poor outcomes. In addition, a significant number of relapsed/refractory patients are ineligible for aggressive therapy because of age, comorbidities, or other factors. While salvage therapies for relapsed or refractory DLBCL have shown encouraging results with respect to rates of response to therapy, long term survival of patients with relapsed or refractory DLBCL remains limited (Lopez et al., (2007) European J of Haematology 80:127-32; Gnaoui et al., (2007) Ann Oncol 18:1363-68; Mounier et al., (2013) Haematologica 98(11)1726-31).

Accordingly, there is a need in the art for new therapeutic approaches in patients with relapsed or refractory DLBCL.

All references cited herein, including patent applications and publications, are hereby incorporated by reference in their entirety.

### SUMMARY

In some aspects, provided herein is a method for treating diffuse large B-cell lymphoma (DLBCL) in a human in need thereof, comprising administering to the human an effective amount of: (a) an immunoconjugate comprising the formula:

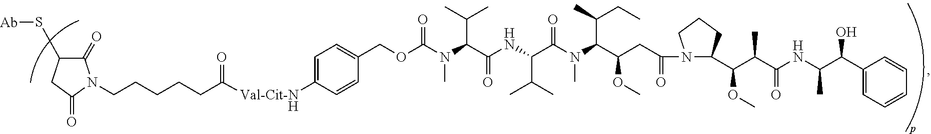

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) an immunomodulatory agent, and (c) an anti-CD20 antibody; and wherein the human achieves at least a complete response during or after treatment with the immunoconjugate, the immunomodulatory agent, and the anti-CD20 antibody. In some embodiments, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response during or after treatment with the immunoconjugate, the immunomodulatory agent, and the anti-CD20 antibody. In some embodiments, among a plurality of humans treated, at least about 70%, at least about 74%, at least about 80%, at least about 90%, or 100% of the humans achieve a best overall response during or after treatment with the immunoconjugate, the immunomodulatory agent, and the anti-CD20 antibody. In some embodiments, among a plurality of humans treated, at least about 30%, at least about 35%, at least about 40%, at least about 50%, at least about 60%, at least about 70%, at least about 80%, at least about 90%, or 100% of the humans achieve a best complete response during or after treatment with the immunoconjugate, the immunomodulatory agent, and the anti-CD20 antibody. In some embodiments, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response during or after treatment with the immunoconjugate, the immunomodulatory agent, and the anti-CD20 antibody. In some embodiments, the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response. In some embodiments, the human survives for at least about 4 months, at least about 5 months, at least about 6 months, at least about 7 months, at least about 8 months, at least about 9 months, at least about 10 months, or more, without disease progression, assessed from the start of treatment with the immunoconjugate, the immunomodulatory agent, and the anti-CD20 antibody. In some embodiments, the human survives for at least about 7 months, at least about 8 months, at least about 9 months, at least about 10 months, at least about 11 months, or more, assessed from the start of treatment with the immunoconjugate, the immunomodulatory agent, and the anti-CD20 antibody.

In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin. In some embodiments, the immunomodulatory agent is lenalidomide. In some embodiments, the anti-CD20 antibody is rituximab. In some embodiments, the polatuzumab vedotin is administered at a dose of about 1.8 mg/kg, the lenalidomide is administered at a dose between about 10 mg and about 20 mg, and the rituximab is administered at a dose of about 375 mg/m².

In some embodiments, the polatuzumab vedotin, the lenalidomide, and the rituximab are administered during an induction phase in 28-day cycles, wherein: the polatuzumab vedotin is administered intravenously at a dose of about 1.8 mg/kg on Day 1 of each 28-day cycle, the lenalidomide is administered orally at a dose between about 10 mg and about 20 mg on each of Days 1-21 of each 28-day cycle, and the rituximab is administered intravenously at a dose of about 375 mg/m² on Day 1 of each 28-day cycle; optionally, wherein the induction phase comprises at least six 28-day cycles. In some embodiments, the polatuzumab vedotin, the lenalidomide, and the rituximab are administered sequentially. In some embodiments, the lenalidomide is administered prior to the rituximab and the rituximab is administered prior to the polatuzumab vedotin on Day 1 of each 28-day cycle. In some embodiments, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response after six 28-day cycles. In some embodiments, among a plurality of humans treated, at least about 70%, at least about 74%, at least about 80%, at least about 90%, or 100% of the humans achieve a best overall response after six 28-day cycles. In some embodiments, among a plurality of humans treated, at least about 30%, at least about 35%, at least about 40%, at least about 50%, at least about 60%, at least about 70%, at least about 80%, at least about 90%, or 100% of the humans achieve a best complete response after six 28-day cycles. In some embodiments, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response after six 28-day cycles. In some embodiments, the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response. In some embodiments, the human survives for at least about 4 months, at least about 5 months, at least about 6 months, at least about 7 months, at least about 8 months, at least about 9 months, at least about 10 months, or more, without disease progression, assessed from the start of treatment with the polatuzumab vedotin, the lenalidomide, and the rituximab. In some embodiments, the human survives for at least about 7 months, at least about 8 months, at least about 9 months, at least about 10 months, at least about 11 months, or more, assessed from the start of treatment with the polatuzumab vedotin, the lenalidomide, and the rituximab.

In some embodiments, the lenalidomide and the rituximab are further administered during a consolidation phase after the sixth 28-day cycle of the induction phase. In some embodiments, the lenalidomide is administered orally at a dose of about 10 mg on each of Days 1-21 of each month during the consolidation phase, and the rituximab is administered intravenously at a dose of about 375 mg/m² on Day 1 of every other month during the consolidation phase. In some embodiments, the lenalidomide is administered for a maximum of 6 months during the consolidation phase. In some embodiments, the rituximab is administered on Day 1 of each of the first, third, and fifth months during the consolidation phase. In some embodiments, the lenalidomide and the rituximab are administered sequentially during the consolidation phase. In some embodiments, the lenalidomide is administered prior to the rituximab on Day 1 of each of the first, third, and fifth months during the consolidation phase.

In another aspect, provided herein is a method for treating diffuse large B-cell lymphoma (DLBCL) in a human in need thereof, comprising administering to the human an effective amount of: (a) an immunoconjugate comprising the formula:

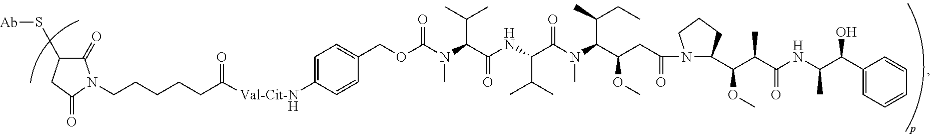

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) an immunomodulatory agent, and (c) an anti-CD20 antibody; and wherein the human does not demonstrate disease progression within at least about 4 months after the start of treatment with the immunoconjugate, the immunomodulatory agent and the anti-CD20 antibody. In some embodiments, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response during or after treatment with the immunoconjugate, the immunomodulatory agent, and the anti-CD20 antibody. In some embodiments, among a plurality of humans treated, at least about 70%, at least about 74%, at least about 80%, at least about 90%, or 100% of the humans achieve a best overall response during or after treatment with the immunoconjugate, the immunomodulatory agent, and the anti-CD20 antibody. In some embodiments, among a plurality of humans treated, at least about 30%, at least about 35%, at least about 40%, at least about 50%, at least about 60%, at least about 70%, at least about 80%, at least about 90%, or 100% of the humans achieve a best complete response during or after treatment with the immunoconjugate, the immunomodulatory agent, and the anti-CD20 antibody. In some embodiments, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response during or after treatment with the immunoconjugate, the immunomodulatory agent, and the anti-CD20 antibody. In some embodiments, the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response. In some embodiments, the human survives for at least about 4 months, at least about 5 months, at least about 6 months, at least about 7 months, at least about 8 months, at least about 9 months, at least about 10 months, or more, without disease progression, assessed from the start of treatment with the immunoconjugate, the immunomodulatory agent, and the anti-CD20 antibody. In some embodiments, the human survives for at least about 7 months, at least about 8 months, at least about 9 months, at least about 10 months, at least about 11 months, or more, assessed from the start of treatment with the immunoconjugate, the immunomodulatory agent, and the anti-CD20 antibody.

In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin. In some embodiments, the immunomodulatory agent is lenalidomide. In some embodiments, the anti-CD20 antibody is rituximab. In some embodiments, the polatuzumab vedotin is administered at a dose of about 1.8 mg/kg, the lenalidomide is administered at a dose between about 10 mg and about 20 mg, and the rituximab is administered at a dose of about 375 mg/m².

In some embodiments, the polatuzumab vedotin, the lenalidomide, and the rituximab are administered during an induction phase in 28-day cycles, wherein: the polatuzumab vedotin is administered intravenously at a dose of about 1.8 mg/kg on Day 1 of each 28-day cycle, the lenalidomide is administered orally at a dose between about 10 mg and about 20 mg on each of Days 1-21 of each 28-day cycle, and the rituximab is administered intravenously at a dose of about 375 mg/m² on Day 1 of each 28-day cycle; optionally, wherein the induction phase comprises at least six 28-day cycles. In some embodiments, the polatuzumab vedotin, the lenalidomide, and the rituximab are administered sequentially. In some embodiments, the lenalidomide is administered prior to the rituximab and the rituximab is administered prior to the polatuzumab vedotin on Day 1 of each 28-day cycle. In some embodiments, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response after six 28-day cycles. In some embodiments, among a plurality of humans treated, at least about 70%, at least about 74%, at least about 80%, at least about 90%, or 100% of the humans achieve a best overall response after six 28-day cycles. In some embodiments, among a plurality of humans treated, at least about 30%, at least about 35%, at least about 40%, at least about 50%, at least about 60%, at least about 70%, at least about 80%, at least about 90%, or 100% of the humans achieve a best complete response after six 28-day cycles. In some embodiments, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response after six 28-day cycles. In some embodiments, the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response. In some embodiments, the human survives for at least about 4 months, at least about 5 months, at least about 6 months, at least about 7 months, at least about 8 months, at least about 9 months, at least about 10 months, or more, without disease progression, assessed from the start of treatment with the polatuzumab vedotin, the lenalidomide, and the rituximab. In some embodiments, the human survives for at least about 7 months, at least about 8 months, at least about 9 months, at least about 10 months, at least about 11 months, or more, assessed from the start of treatment with the polatuzumab vedotin, the lenalidomide, and the rituximab.

In some embodiments, the lenalidomide and the rituximab are further administered during a consolidation phase after the sixth 28-day cycle of the induction phase. In some embodiments, the lenalidomide is administered orally at a dose of about 10 mg on each of Days 1-21 of each month during the consolidation phase, and the rituximab is administered intravenously at a dose of about 375 mg/m² on Day 1 of every other month during the consolidation phase. In some embodiments, the lenalidomide is administered for a maximum of 6 months during the consolidation phase. In some embodiments, the rituximab is administered on Day 1 of each of the first, third, and fifth months during the consolidation phase. In some embodiments, the lenalidomide and the rituximab are administered sequentially during the consolidation phase. In some embodiments, the lenalidomide is administered prior to the rituximab on Day 1 of each of the first, third, and fifth months during the consolidation phase.

In another aspect, provided herein is a method of treating diffuse large B-cell lymphoma (DLBCL) in a human in need thereof, comprising administering to the human an effective amount of: (a) an immunoconjugate comprising the formula:

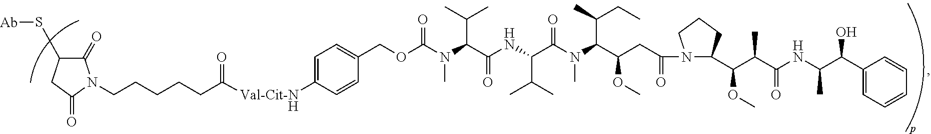

wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, (b) lenalidomide and (c) rituximab, wherein the immunoconjugate is administered at a dose of about 1.8 mg/kg, the lenalidomide is administered at a dose between about 10 mg and about 20 mg, and the rituximab is administered at a dose of about 375 mg/m², and wherein the human achieves at least a complete response during or after treatment with the immunoconjugate, the lenalidomide, and the rituximab. In some embodiments, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response during or after treatment with the immunoconjugate, the lenalidomide, and the rituximab. In some embodiments, among a plurality of humans treated, at least about 70%, at least about 74%, at least about 80%, at least about 90%, or 100% of the humans achieve a best overall response during or after treatment with the immunoconjugate, the lenalidomide, and the rituximab. In some embodiments, among a plurality of humans treated, at least about 30%, at least about 35%, at least about 40%, at least about 50%, at least about 60%, at least about 70%, at least about 80%, at least about 90%, or 100% of the humans achieve a best complete response during or after treatment with the immunoconjugate, the lenalidomide, and the rituximab. In some embodiments, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response during or after treatment with the immunoconjugate, the lenalidomide, and the rituximab. In some embodiments, the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response. In some embodiments, the human survives for at least about 4 months, at least about 5 months, at least about 6 months, at least about 7 months, at least about 8 months, at least about 9 months, at least about 10 months, or more, without disease progression, assessed from the start of treatment with the immunoconjugate, the lenalidomide, and the rituximab. In some embodiments, the human survives for at least about 7 months, at least about 8 months, at least about 9 months, at least about 10 months, at least about 11 months, or more, assessed from the start of treatment with the immunoconjugate, the lenalidomide, and the rituximab.

In some embodiments, p is between 3 and 4. In some embodiments, the antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin.

In some embodiments, the polatuzumab vedotin, the lenalidomide, and the rituximab are administered during an induction phase in 28-day cycles, wherein: the polatuzumab vedotin is administered intravenously at a dose of about 1.8 mg/kg on Day 1 of each 28-day cycle, the lenalidomide is administered orally at a dose between about 10 mg and about 20 mg on each of Days 1-21 of each 28-day cycle, and the rituximab is administered intravenously at a dose of about 375 mg/m² on Day 1 of each 28-day cycle; optionally, wherein the induction phase comprises at least six 28-day cycles. In some embodiments, the polatuzumab vedotin, the lenalidomide, and the rituximab are administered sequentially. In some embodiments, the lenalidomide is administered prior to the rituximab and the rituximab is administered prior to the polatuzumab vedotin on Day 1 of each 28-day cycle. In some embodiments, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response after six 28-day cycles. In some embodiments, among a plurality of humans treated, at least about 70%, at least about 74%, at least about 80%, at least about 90%, or 100% of the humans achieve a best overall response after six 28-day cycles. In some embodiments, among a plurality of humans treated, at least about 30%, at least about 35%, at least about 40%, at least about 50%, at least about 60%, at least about 70%, at least about 80%, at least about 90%, or 100% of the humans achieve a best complete response after six 28-day cycles. In some embodiments, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response after six 28-day cycles. In some embodiments, the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response. In some embodiments, the human survives for at least about 4 months, at least about 5 months, at least about 6 months, at least about 7 months, at least about 8 months, at least about 9 months, at least about 10 months, or more, without disease progression, assessed from the start of treatment with the polatuzumab vedotin, the lenalidomide, and the rituximab. In some embodiments, the human survives for at least about 7 months, at least about 8 months, at least about 9 months, at least about 10 months, at least about 11 months, or more, assessed from the start of treatment with the polatuzumab vedotin, the lenalidomide, and the rituximab.

In some embodiments, the lenalidomide and the rituximab are further administered during a consolidation phase after the sixth 28-day cycle of the induction phase. In some embodiments, the lenalidomide is administered orally at a dose of about 10 mg on each of Days 1-21 of each month during the consolidation phase, and the rituximab is administered intravenously at a dose of about 375 mg/m² on Day 1 of every other month during the consolidation phase. In some embodiments, the lenalidomide is administered for a maximum of 6 months during the consolidation phase. In some embodiments, the rituximab is administered on Day 1 of each of the first, third, and fifth months during the consolidation phase. In some embodiments, the lenalidomide and the rituximab are administered sequentially during the consolidation phase. In some embodiments, the lenalidomide is administered prior to the rituximab on Day 1 of each of the first, third, and fifth months during the consolidation phase.

In another aspect, provided herein is a method of treating diffuse large B-cell lymphoma (DLBCL) in a human in need thereof, comprising administering to the human an effective amount of: (a) polatuzumab vedotin; (b) lenalidomide; and (c) rituximab, during an induction phase in 28-day cycles, wherein, during the induction phase, the polatuzumab vedotin is administered at a dose of about 1.8 mg/kg, the lenalidomide is administered at a dose of about 20 mg, and the rituximab is administered at a dose of about 375 mg/m², and wherein the human achieves a complete response during or after the induction phase. In some embodiments, the induction phase comprises at least six 28-day cycles. In some embodiments, the polatuzumab vedotin is administered intravenously at a dose of about 1.8 mg/kg on Day 1 of each 28-day cycle, the lenalidomide is administered orally at a dose of about 20 mg on each of Days 1-21 of each 28-day cycle, and the rituximab is administered intravenously at a dose of about 375 mg/m² on Day 1 of each 28-day cycle. In some embodiments, the polatuzumab vedotin, the lenalidomide, and the rituximab are administered sequentially. In some embodiments, the lenalidomide is administered prior to the rituximab and the rituximab is administered prior to the polatuzumab vedotin on Day 1 of each 28-day cycle. In some embodiments, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response after six 28-day cycles. In some embodiments, among a plurality of humans treated, at least about 70%, at least about 74%, at least about 80%, at least about 90%, or 100% of the humans achieve a best overall response after six 28-day cycles. In some embodiments, among a plurality of humans treated, at least about 30%, at least about 35%, at least about 40%, at least about 50%, at least about 60%, at least about 70%, at least about 80%, at least about 90%, or 100% of the humans achieve a best complete response after six 28-day cycles. In some embodiments, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response after six 28-day cycles. In some embodiments, the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response. In some embodiments, the human survives for at least about 4 months, at least about 5 months, at least about 6 months, at least about 7 months, at least about 8 months, at least about 9 months, at least about 10 months, or more, without disease progression, assessed from the start of treatment with the polatuzumab vedotin, the lenalidomide, and the rituximab. In some embodiments, the human survives for at least about 7 months, at least about 8 months, at least about 9 months, at least about 10 months, at least about 11 months, or more, assessed from the start of treatment with the polatuzumab vedotin, the lenalidomide, and the rituximab.

In some embodiments, the induction phase is followed by a consolidation phase, wherein the lenalidomide is administered at a dose of about 10 mg and the rituximab is administered at a dose of about 375 mg/m² during the consolidation phase. In some embodiments, the lenalidomide is administered orally at a dose of about 10 mg on each of Days 1-21 of each month during the consolidation phase, and the rituximab is administered intravenously at a dose of about 375 mg/m² on Day 1 of every other month during the consolidation phase. In some embodiments, the lenalidomide is administered for a maximum of 6 months during the consolidation phase. In some embodiments, the rituximab is administered on Day 1 of each of the first, third, and fifth months during the consolidation phase. In some embodiments, the lenalidomide and the rituximab are administered sequentially during the consolidation phase. In some embodiments, the lenalidomide is administered prior to the rituximab on Day 1 of each of the first, third, and fifth months during the consolidation phase.

In another aspect, provided herein is a method of treating diffuse large B-cell lymphoma (DLBCL) in a plurality of humans in need thereof, comprising administering to the humans an effective amount of: (a) polatuzumab vedotin; (b) lenalidomide; and (c) rituximab, during an induction phase in 28-day cycles, wherein, during the induction phase, the polatuzumab vedotin is administered at a dose of about 1.8 mg/kg, the lenalidomide is administered at a dose of about 20 mg, and the rituximab is administered at a dose of about 375 mg/m², and wherein, at least about 25% of the humans in the plurality achieve a complete response during or after the induction phase. In some embodiments, the induction phase comprises at least six 28-day cycles. In some embodiments, the polatuzumab vedotin is administered intravenously at a dose of about 1.8 mg/kg on Day 1 of each 28-day cycle, the lenalidomide is administered orally at a dose of about 20 mg on each of Days 1-21 of each 28-day cycle, and the rituximab is administered intravenously at a dose of about 375 mg/m² on Day 1 of each 28-day cycle. In some embodiments, the polatuzumab vedotin, the lenalidomide, and the rituximab are administered sequentially. In some embodiments, the lenalidomide is administered prior to the rituximab and the rituximab is administered prior to the polatuzumab vedotin on Day 1 of each 28-day cycle. In some embodiments, at least about 27%, at least about 29%, at least about 30%, at least about 40%, at least about 50%, at least about 60%, at least about 70%, at least about 80%, at least about 90%, or 100% of the humans in the plurality achieve a complete response after six 28-day cycles. In some embodiments, at least about 70%, at least about 74%, at least about 80%, at least about 90%, or 100% of the humans in the plurality achieve a best overall response after six 28-day cycles. In some embodiments, at least about 30%, at least about 35%, at least about 40%, at least about 50%, at least about 60%, at least about 70%, at least about 80%, at least about 90%, or 100% of the humans in the plurality achieve a best complete response after six 28-day cycles. In some embodiments, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response after six 28-day cycles. In some embodiments, the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response.

In some embodiments, the induction phase is followed by a consolidation phase, wherein the lenalidomide is administered at a dose of about 10 mg and the rituximab is administered at a dose of about 375 mg/m² during the consolidation phase. In some embodiments, the lenalidomide is administered orally at a dose of about 10 mg on each of Days 1-21 of each month during the consolidation phase, and the rituximab is administered intravenously at a dose of about 375 mg/m² on Day 1 of every other month during the consolidation phase. In some embodiments, the lenalidomide is administered for a maximum of 6 months during the consolidation phase. In some embodiments, the rituximab is administered on Day 1 of each of the first, third, and fifth months during the consolidation phase. In some embodiments, the lenalidomide and the rituximab are administered sequentially during the consolidation phase. In some embodiments, the lenalidomide is administered prior to the rituximab on Day 1 of each of the first, third, and fifth months during the consolidation phase.

In some embodiments of any of the aspects or embodiments provided herein, the human or a human in the plurality of humans has received at least one prior therapy for DLBCL. In some embodiments, the human or a human in the plurality of humans has received at least two prior therapies for DLBCL. In some embodiments, the human or a human in the plurality of humans has received a prior therapy for DLBCL comprising a chemoimmunotherapy that included an anti-CD20 antibody. In some embodiments, the human or a human in the plurality of humans has been administered a prior bone marrow transplant for DLBCL. In some embodiments, the human or a human in the plurality of humans has been administered a prior chimeric antigen receptor (CAR)-T-cell therapy for DLBCL. In some embodiments, the human or a human in the plurality of humans has DLBCL that was refractory to the first prior treatment for DLBCL administered to the human or the human in the plurality of humans. In some embodiments, the human or a human in the plurality of humans has DLBCL that was refractory to the most recent prior therapy for DLBCL. In some embodiments, the DLBCL is relapsed/refractory DLBCL. In some embodiments, the DLBCL is relapsed/refractory DLBCL after treatment with at least one prior chemoimmunotherapy regimen that included an anti-CD20 antibody. In some embodiments, the human or a human in the plurality of humans experienced disease progression after treatment with high-dose chemotherapy and autologous stem-cell transplantation. In some embodiments, the DLBCL is CD20-positive DLBCL. In some embodiments, the DLBCL is a positron emission tomography (PET)-positive lymphoma. In some embodiments, the human or a human in the plurality of humans is not eligible for autologous stem-cell transplantation. In some embodiments, the human or a human in the plurality of humans does not have central nervous system (CNS) lymphoma or leptomeningeal infiltration. In some embodiments, the human or a human in the plurality of humans has at least one bi-dimensionally measurable lesion. In some embodiments, the at least one bi-dimensionally measurable lesion is greater than 1.5 cm in its largest dimension, assessed by computed tomography (CT) scan or magnetic resonance imaging (MRI). In some embodiments, the human or a human in the plurality of humans has not received a prior allogenic stem cell transplantation (SCT). In some embodiments, the human or a human in the plurality of humans does not have history of transformation of indolent disease to DLBCL. In some embodiments, the human or a human in the plurality of humans does not have Grade 2 or greater neuropathy. In some embodiments, the human or a human in the plurality of humans has an Eastern Cooperative Oncology Group (ECOG) Performance Status of 0, 1, or 2. In some embodiments, the human or a human in the plurality of humans has DLBCL with an Ann Arbor Stage III or IV. In some embodiments, the human or a human in the plurality of humans has DLBCL with an International Prognostic Index of between 3 and 5.

In another aspect, provided herein is a kit comprising an immunoconjugate comprising the formula:

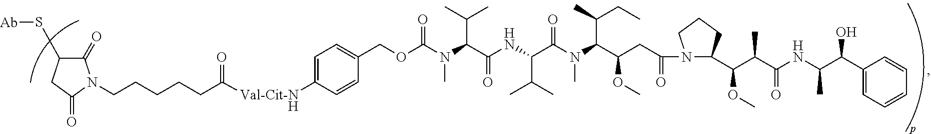

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with an immunomodulatory agent and an anti-CD20 antibody for treating a human in need thereof having diffuse large B-cell lymphoma (DLBCL) according to any of the methods provided herein. In some embodiments, p is between 3 and 4. In some embodiments, the antibody comprises (i) a heavy chain comprising the amino acid sequence of SEQ ID NO: 36 and (ii) a light chain comprising the amino acid sequence of SEQ ID NO: 35. In some embodiments, the DLBCL is relapsed/refractory DLBCL.

In another aspect, provided herein is a kit comprising an immunoconjugate comprising the formula:

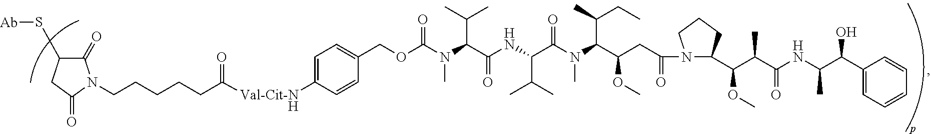

wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, for use in combination with lenalidomide and rituximab for treating a human in need thereof having diffuse large B-cell lymphoma (DLBCL) according to any of the methods provided herein. In some embodiments, p is between 3 and 4. In some embodiments, the antibody comprises (i) a heavy chain comprising the amino acid sequence of SEQ ID NO: 36 and (ii) a light chain comprising the amino acid sequence of SEQ ID NO: 35. In some embodiments, the DLBCL is relapsed/refractory DLBCL.

In another aspect, provided herein is a kit comprising polatuzumab vedotin for use in combination with lenalidomide and rituximab for treating a human in need thereof having diffuse large B-cell lymphoma (DLBCL) according to any of the methods provided herein. In some embodiments, the DLBCL is relapsed/refractory DLBCL.

In another aspect, provided herein is an immunoconjugate comprising the formula:

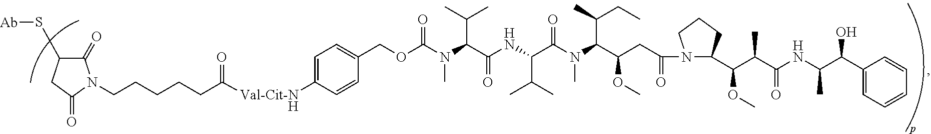

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in a method of treating diffuse large B-cell lymphoma (DLBCL) according to any of the methods provided herein. In some embodiments, the anti-CD79b antibody comprises (i) a heavy chain variable domain (VH) that comprises the amino acid sequence of SEQ ID NO: 19 and (ii) a light chain variable domain (VL) that comprises the amino acid sequence of SEQ ID NO: 20. In some embodiments, p is between 3 and 4. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the DLBCL is relapsed/refractory DLBCL.

In another aspect, provided herein is an immunoconjugate comprising the formula:

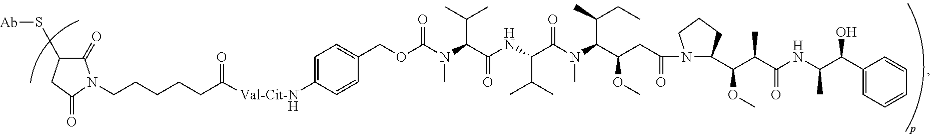

wherein Ab is an anti-CD79b antibody that comprises (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, for use in a method of treating diffuse large B-cell lymphoma (DLBCL) according to any one of the methods provided herein. In some embodiments, p is between 3 and 4. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the DLBCL is relapsed/refractory DLBCL.

In another aspect, provided herein is polatuzumab vedotin for use in a method of treating diffuse large B-cell lymphoma (DLBCL) according to any of the methods provided herein. In some embodiments, the DLBCL is relapsed/refractory DLBCL.

### BRIEF DESCRIPTION OF THE DRAWINGS

FIG. 1 is a diagram of the study design of the Phase Ib/II study described in Example 1. C=cycle; CR=complete response; D=day; DLBCL=diffuse large B-cell lymphoma; EOI=end of induction; Len=lenalidomide; PO=by mouth; Pola=polatuzumab vedotin; PR=partial response; QD=every day; Q2M=every 2 months; R=rituximab; RP2D=recommended Phase II dose; SD=stable disease.

FIG. 2 is a diagram of the 3+3 dose-escalation schema used during the dose escalation phase of the Phase Ib/II study described in Example 1.

FIG. 3 is a diagram providing an overview of the dosing regimens used in the Phase Ib/II study described in Example 1.

FIG. 4 provides an overview of the study design of the Phase Ib/II study described in Examples 1 and 2. CR, complete response; IV, intravenous; Len, lenalidomide; PO, oral; Pola, polatuzumab vedotin; PR, partial response; R, rituximab; RP2D, recommended phase II dose.

FIG. 5 provides an overview of the study populations in the primary analysis of the Phase Ib/II study described in Example 2. RP2D=recommended Phase II dose.

FIG. 6 is a Swimlane plot showing the time to response and duration of response for patients evaluated in the primary analysis of the Phase Ib/II study described in Example 2. Interim responses were assessed by CT according to Lugano 2014 criteria. EOI responses were assessed by PET-CT according to Modified Lugano 2014 criteria.

FIG. 7 provides Kaplan-Meier survival curves for progression-free survival (PFS) and overall survival (OS).

### DETAILED DESCRIPTION

As used herein, the term “polatuzumab vedotin” refers to an anti-CD79b immunoconjugate having the IUPHAR/BPS Number 8404, the KEGG Number D10761, or the CAS Registry Number 1313206-42-6. Polatuzumab vedotin is also interchangeably referred to as “polatuzumab vedotin-piiq”, “huMA79bv28-MC-vc-PAB-MMAE”, “DCDS4501A”, or “RG7596.”

Provided herein are methods for treating or delaying progression of lymphoma (such as diffuse large B-cell lymphoma (DLBCL), e.g., relapsed/refractory DLBCL) in an individual (e.g., a human), comprising administering to the individual an effective amount of an anti-CD79b immunoconjugate (e.g., huMA79bv28-MC-vc-PAB-MMAE, which is also known as polatuzumab vedotin), an immunomodulatory agent (e.g., lenalidomide), and an anti-CD20 agent (e.g., an anti-CD20 antibody such as obinutuzumab or rituximab). In some embodiments, the methods comprise treating an individual having diffuse large B-cell lymphoma (DLBCL), e.g., relapsed/refractory DLBCL, by administering to the individual: (a) an immunoconjugate comprising the formula:

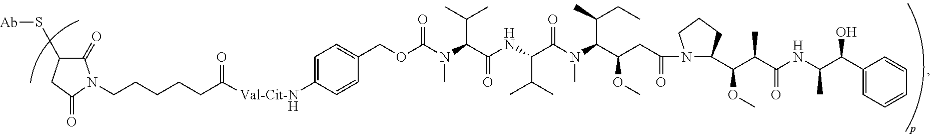

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 (e.g., between 2 and 5, or between 3 and 4), (b) an immunomodulatory agent (e.g., lenalidomide), and (c) an anti-CD20 antibody (e.g., obinutuzumab or rituximab). In some embodiments, the immunoconjugate is administered at a dose between about 1.4 mg/kg and about 1.8 mg/kg, the immunomodulatory agent (e.g., lenalidomide) is administered at a dose between about 10 mg and about 20 mg, and the anti-CD20 antibody (e.g., rituximab) is administered at a dose of about 375 mg/m². In some embodiments, the immunoconjugate is administered at a dose between about 1.4 mg/kg and about 1.8 mg/kg, the immunomodulatory agent (e.g., lenalidomide) is administered at a dose between about 10 mg and about 20 mg, and the anti-CD20 antibody (e.g., obinutuzumab) is administered at a dose of about 1000 mg. In some embodiments, the individual achieves a response of at least stable disease (SD) (such as at least SD, at least partial response (PR), or a complete response/complete remission (CR)) during or after treatment with the immunoconjugate, the immunomodulatory agent, and the anti-CD20 antibody.
In some embodiments, the individual achieves an objective response, a best overall response, a best complete response, a best partial response, or a complete response during or after treatment with the immunoconjugate, the immunomodulatory agent, and the anti-CD20 antibody.

### I. General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

### II. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The term “CD79b,” as used herein, refers to any native CD79b from any vertebrate source, including mammals such as primates (e.g., humans, cynomologus monkey (“cyno”)) and rodents (e.g., mice and rats), unless otherwise indicated. Human CD79b is also referred to herein as “Igβ,” “B29,” “DNA225786” or “PRO36249.” An exemplary CD79b sequence including the signal sequence is shown in SEQ ID NO: 1. An exemplary CD79b sequence without the signal sequence is shown in SEQ ID NO: 2. The term “CD79b” encompasses “full-length,” unprocessed CD79b as well as any form of CD79b that results from processing in the cell. The term also encompasses naturally occurring variants of CD79b, e.g., splice variants, allelic variants and isoforms. The CD79b polypeptides described herein may be isolated from a variety of sources, such as from human tissue types or from another source, or prepared by recombinant or synthetic methods. A “native sequence CD79b polypeptide” comprises a polypeptide having the same amino acid sequence as the corresponding CD79b polypeptide derived from nature. Such native sequence CD79b polypeptides can be isolated from nature or can be produced by recombinant or synthetic means. The term “native sequence CD79b polypeptide” specifically encompasses naturally-occurring truncated or secreted forms of the specific CD79b polypeptide (e.g., an extracellular domain sequence), naturally-occurring variant forms (e.g., alternatively spliced forms) and naturally-occurring allelic variants of the polypeptide.

“CD20” as used herein refers to the human B-lymphocyte antigen CD20 (also known as CD20, B-lymphocyte surface antigen B1, Leu-16, Bp35, BM5, and LF5; the sequence is characterized by the SwissProt database entry P11836) is a hydrophobic transmembrane protein with a molecular weight of approximately 35 kD located on pre-B and mature B lymphocytes. (Valentine, M. A., et al., J. Biol. Chem. 264(19) (1989 11282-11287; Tedder, T. F., et al, Proc. Natl. Acad. Sci. U.S.A. 85 (1988) 208-12; Stamenkovic, I., et al., J. Exp. Med. 167 (1988) 1975-80; Einfeld, D. A. et al., EMBO J. 7 (1988) 711-7; Tedder, T. F., et al., J. Immunol. 142 (1989) 2560-8). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein.

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediates the killing of cells expressing CD20 (e.g., a tumor cell) by inactivating CD20. The killing of the cells expressing CD20 may occur by one or more of the following mechanisms: Cell death/apoptosis induction, ADCC and CDC. Synonyms of CD20, as recognized in the art, include B-lymphocyte antigen CD20, B-lymphocyte surface antigen B1, Leu-16, Bp35, BM5, and LF5.

The term “expression of the CD20” antigen is intended to indicate a significant level of expression of the CD20 antigen in a cell, e.g., a T- or B-Cell. In one embodiment, patients to be treated according to the methods of this invention express significant levels of CD20 on a B-cell tumor or cancer. Patients having a “CD20 expressing cancer” can be determined by standard assays known in the art. E.g., CD20 antigen expression is measured using immunohistochemical (IHC) detection, FACS or via PCR-based detection of the corresponding mRNA.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” herein is used in the broadest sense and encompasses various antibody structures, including but not limited to, monoclonal antibodies, polyclonal antibodies, multispecific antibodies (e.g., bispecific antibodies), and antibody fragments, so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “epitope” refers to the particular site on an antigen molecule to which an antibody binds.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “anti-CD79b antibody” or “an antibody that binds to CD79b” refers to an antibody that is capable of binding CD79b with sufficient affinity such that the antibody is useful as a diagnostic and/or therapeutic agent in targeting CD79b. Preferably, the extent of binding of an anti-CD79b antibody to an unrelated, non-CD79b protein is less than about 10% of the binding of the antibody to CD79b as measured, e.g., by a radioimmunoassay (RIA). In certain embodiments, an antibody that binds to CD79b has a dissociation constant (Kd) of ≤1 μM, ≤100 nM, ≤10 nM, ≤1 nM, or ≤0.1 nM. In certain embodiments, an anti-CD79b antibody binds to an epitope of CD79b that is conserved among CD79b from different species.

The term “anti-CD20 antibody” according to the invention refers to an antibody that is capable of binding CD20 with sufficient affinity such that the antibody is useful as a diagnostic and/or therapeutic agent in targeting CD20. Preferably, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of ≤1 μM, ≤100 nM, ≤10 nM, ≤1 nM, or ≤0.1 nM. In certain embodiments, anti-CD20 antibody binds to an epitope of CD20 that is conserved among CD20 from different species.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For review of methods for assessment of antibody purity, see, e.g., Flatman et al., J. Chromatogr. B 848:79-87 (2007). The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

“Isolated nucleic acid encoding an anti-CD79b antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell, or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat et al., Sequences of Proteins of Immunological Interest, 5th Ed. Public Health Service, National Institutes of Health, Bethesda, MD (1991). With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008). Unless otherwise indicated, HVR residues and other residues in the variable domain (e.g., FR residues) are numbered herein according to Kabat et al., supra.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs) (See, e.g., Kindt et al. Kuby Immunology, 6^{th} ed., W.H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B-cell receptor); and B-cell activation.

“CD79b polypeptide variant” means a CD79b polypeptide, preferably an active CD79b polypeptide, as defined herein having at least about 80% amino acid sequence identity with a full-length native sequence CD79b polypeptide sequence as disclosed herein, a CD79b polypeptide sequence lacking the signal peptide as disclosed herein, an extracellular domain of a CD79b polypeptide, with or without the signal peptide, as disclosed herein or any other fragment of a full-length CD79b polypeptide sequence as disclosed herein (such as those encoded by a nucleic acid that represents only a portion of the complete coding sequence for a full-length CD79b polypeptide). Such CD79b polypeptide variants include, for instance, CD79b polypeptides wherein one or more amino acid residues are added, or deleted, at the N- or C-terminus of the full-length native amino acid sequence. Ordinarily, a CD79b polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence CD79b polypeptide sequence as disclosed herein, a CD79b polypeptide sequence lacking the signal peptide as disclosed herein, an extracellular domain of a CD79b polypeptide, with or without the signal peptide, as disclosed herein or any other specifically defined fragment of a full-length CD79b polypeptide sequence as disclosed herein. Ordinarily, CD79b variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, CD79b variant polypeptides will have no more than one conservative amino acid substitution as compared to the native CD79b polypeptide sequence, alternatively no more than 2, 3, 4, 5, 6, 7, 8, 9, or 10 conservative amino acid substitution as compared to the native CD79b polypeptide sequence.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including but not limited to a cytotoxic agent.

In the context of the formulas provided herein, “p” refers to the average number of drug moieties per antibody, which can range, e.g., from about 1 to about 20 drug moieties per antibody, and in certain embodiments, from 1 to about 8 drug moieties per antibody. The invention includes a composition comprising a mixture of antibody-drug compounds of Formula I where the average drug loading per antibody is about 2 to about 5, or about 3 to about 4, (e.g., about 3.4 or about 3.5).

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The terms “cancer” and “cancerous” refer to or describe the physiological condition in mammals that is typically characterized by unregulated cell growth. Examples of cancer include but are not limited to, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), follicular lymphoma (e.g., relapsed/refractory follicular lymphoma), intermediate grade diffuse NHL, diffuse large B-cell lymphoma (DLBCL; e.g., relapsed/refractory DLBCL), aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “effective amount” of an agent, e.g., a pharmaceutical formulation, refers to an amount effective, at dosages and for periods of time necessary, to achieve the desired therapeutic or prophylactic result.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, reduction of free light chain, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, the methods described herein are used to delay development of a disease or to slow the progression of a disease.

The term “CD79b-positive cancer” refers to a cancer comprising cells that express CD79b on their surface. In some embodiments, expression of CD79b on the cell surface is determined, for example, using antibodies to CD79b in a method such as immunohistochemistry, FACS, etc. Alternatively, CD79b mRNA expression is considered to correlate to CD79b expression on the cell surface and can be determined by a method selected from in situ hybridization and RT-PCR (including quantitative RT-PCR).

As used herein, “in conjunction with” refers to administration of one treatment modality in addition to another treatment modality. As such, “in conjunction with” refers to administration of one treatment modality before, during, or after administration of the other treatment modality to the individual.

A “chemotherapeutic agent” is a chemical compound useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, everolimus, sotrataurin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. Additional examples of chemotherapeutic agents include bendamustine (or bendamustine-HCl) (TREANDA®), ibrutinib, lenalidomide, and/or idelalisib (GS-1101).

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine.

In some embodiments, the chemotherapeutic agent includes a topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

Chemotherapeutic agents also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ublituximab, ofatumumab, ibritumomab tiuxetan, pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds or agents of the disclosure include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1λ antibody genetically modified to recognize interleukin-12 p40 protein.

The term “package insert” is used to refer to instructions customarily included in commercial packages of therapeutic products, that contain information about the indications, usage, dosage, administration, combination therapy, contraindications and/or warnings concerning the use of such therapeutic products.

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃) 3), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The term “C₁-C₅ alkyl,” as used herein refers to a straight chain or branched, saturated or unsaturated hydrocarbon having from 1 to 8 carbon atoms. Representative “C1-C8 alkyl” groups include, but are not limited to, -methyl, -ethyl, -n-propyl, -n-butyl, -n-pentyl, -n-hexyl, -n-heptyl, -n-octyl, -n-nonyl and -n-decyl; while branched C₁-C₅ alkyls include, but are not limited to, -isopropyl, -sec-butyl, -isobutyl, -tert-butyl, -isopentyl, 2-methylbutyl, unsaturated C₁-C₅ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, -3-methyl-1 butynyl. A C₁-C₅ alkyl group can be unsubstituted or substituted with one or more groups including, but not limited to, —C₁-C₈ alkyl, —O—(C₁-C₅ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

The term “C₁-C₁₂ alkyl,” as used herein refers to a straight chain or branched, saturated or unsaturated hydrocarbon having from 1 to 12 carbon atoms. A C₁-C₁₂ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

The term “C₁-C₆ alkyl,” as used herein refers to a straight chain or branched, saturated or unsaturated hydrocarbon having from 1 to 6 carbon atoms. Representative “C₁-C₆ alkyl” groups include, but are not limited to, -methyl, -ethyl, -n-propyl, -n-butyl, -n-pentyl, -and n-hexyl; while branched C₁-C₆ alkyls include, but are not limited to, -isopropyl, -sec-butyl, -isobutyl, -tert-butyl, -isopentyl, and 2-methylbutyl; unsaturated C₁-C₆ alkyls include, but are not limited to, -vinyl, -allyl, -1-butenyl, -2-butenyl, and -isobutylenyl, -1-pentenyl, -2-pentenyl, -3-methyl-1-butenyl, -2-methyl-2-butenyl, -2,3-dimethyl-2-butenyl, 1-hexyl, 2-hexyl, and 3-hexyl. A C₁-C₆ alkyl group can be unsubstituted or substituted with one or more groups, as described above for C₁-C₈ alkyl group.

The term “C₁-C₄ alkyl,” as used herein refers to a straight chain or branched, saturated or unsaturated hydrocarbon having from 1 to 4 carbon atoms. Representative “C₁-C₄ alkyl” groups include, but are not limited to, -methyl, -ethyl, -n-propyl, -n-butyl; while branched C₁-C₄ alkyls include, but are not limited to, -isopropyl, -sec-butyl, -isobutyl, -tert-butyl; unsaturated C₁-C₄ alkyls include, but are not limited to, -vinyl, -allyl, -1-butenyl, -2-butenyl, and -isobutylenyl. A C₁-C₄ alkyl group can be unsubstituted or substituted with one or more groups, as described above for C₁-C₈ alkyl group.

“Alkoxy” is an alkyl group singly bonded to an oxygen. Exemplary alkoxy groups include, but are not limited to, methoxy (—OCH₃) and ethoxy (—OCH₂CH₃). A “C₁-C₈ alkoxy” is an alkoxy group with 1 to 5 carbon atoms. Alkoxy groups may can be unsubstituted or substituted with one or more groups, as described above for alkyl groups.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H7), and 5-hexenyl (—CH₂ CH₂CH₂CH₂CH═CH₂). A “C₂-C₅ alkenyl” is a hydrocarbon containing 2 to 8 normal, secondary, tertiary or cyclic carbon atoms with at least one site of unsaturation, i.e. a carbon-carbon, sp² double bond.

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH). A “C₂-C₅ alkynyl” is a hydrocarbon containing 2 to 8 normal, secondary, tertiary or cyclic carbon atoms with at least one site of unsaturation, i.e. a carbon-carbon, sp triple bond.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

A “C₁-C₁₀ alkylene” is a straight chain, saturated hydrocarbon group of the formula —(CH₂)₁₋₁₀—. Examples of a C₁-C₁₀ alkylene include methylene, ethylene, propylene, butylene, pentylene, hexylene, heptylene, ocytylene, nonylene and decalene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene (—C≡C—), propargyl (—CH₂C≡C—), and 4-pentynyl (—CH₂CH₂CH₂C≡C—).

“Aryl” refers to a carbocyclic aromatic group. Examples of aryl groups include, but are not limited to, phenyl, naphthyl and anthracenyl. A carbocyclic aromatic group or a heterocyclic aromatic group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

A “C₅-C₂₀ aryl” is an aryl group with 5 to 20 carbon atoms in the carbocyclic aromatic rings. Examples of C₅-C₂₀ aryl groups include, but are not limited to, phenyl, naphthyl and anthracenyl. A C₅-C₂₀ aryl group can be substituted or unsubstituted as described above for aryl groups. A “C₅-C₁₄ aryl” is an aryl group with 5 to 14 carbon atoms in the carbocyclic aromatic rings. Examples of C₅-C₁₄ aryl groups include, but are not limited to, phenyl, naphthyl and anthracenyl. A C₅-C₁₄ aryl group can be substituted or unsubstituted as described above for aryl groups.

An “arylene” is an aryl group which has two covalent bonds and can be in the ortho, meta, or para configurations as shown in the following structures:

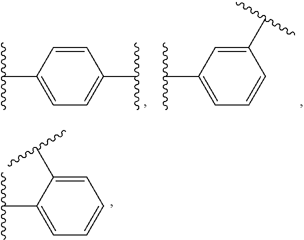

in which the phenyl group can be unsubstituted or substituted with up to four groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo [4,5], [5,5], [5,6], or [6,6] system.

“Substituted alkyl,” “substituted aryl,” and “substituted arylalkyl” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃⁻, —SO₃H, —S(═O)₂R, —OS(═O)₂OR, —S(═O)₂NR, —S(═O)R, —OP(═O)(OR)₂, —P(═O)(OR)₂, —PO⁻₃, —PO₃H₂, —C(═O)R, —C(═O)X, —C(═S)R, —CO₂R, —CO₂⁻, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently —H, C₂-C₁₈ alkyl, C₆-C₂₀ aryl, C₃-C₁₄ heterocycle, protecting group or prodrug moiety. Alkylene, alkenylene, and alkynylene groups as described above may also be similarly substituted.

“Heteroaryl” and “heterocycle” refer to a ring system in which one or more ring atoms is a heteroatom, e.g., nitrogen, oxygen, and sulfur. The heterocycle radical comprises 3 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system.

Exemplary heterocycles are described, e.g., in Paquette, Leo A., “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

A “C₃-C₈ heterocycle” refers to an aromatic or non-aromatic C₃-C₈ carbocycle in which one to four of the ring carbon atoms are independently replaced with a heteroatom from the group consisting of O, S and N. Representative examples of a C₃-C₈ heterocycle include, but are not limited to, benzofuranyl, benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A C₃-C₈ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₈ heterocyclo” refers to a C₃-C₈ heterocycle group defined above wherein one of the heterocycle group's hydrogen atoms is replaced with a bond. A C₃-C₈ heterocyclo can be unsubstituted or substituted with up to six groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

A “C₃-C₂₀ heterocycle” refers to an aromatic or non-aromatic C₃-C₈ carbocycle in which one to four of the ring carbon atoms are independently replaced with a heteroatom from the group consisting of O, S and N. A C₃-C₂₀ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₂₀ heterocyclo” refers to a C₃-C₂₀ heterocycle group defined above wherein one of the heterocycle group's hydrogen atoms is replaced with a bond.

“Carbocycle” means a saturated or unsaturated ring having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cycloheptyl, and cyclooctyl.

A “C₃-C₈ carbocycle” is a 3-, 4-, 5-, 6-, 7- or 8-membered saturated or unsaturated non-aromatic carbocyclic ring. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

A “C₃-C₈ carbocyclo” refers to a C₃-C₈ carbocycle group defined above wherein one of the carbocycle groups' hydrogen atoms is replaced with a bond.

“Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)ₙO(CR₂)ₙ—, repeating units of alkyloxy (e.g., polyethylenoxy, PEG, polymethyleneoxy) and alkylamino (e.g., polyethyleneamino, Jeffamine™); and diacid ester and amides including succinate, succinamide, diglycolate, malonate, and caproamide. In various embodiments, linkers can comprise one or more amino acid residues, such as valine, phenylalanine, lysine, and homolysine.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Leaving group” refers to a functional group that can be substituted by another functional group. Certain leaving groups are well known in the art, and examples include, but are not limited to, a halide (e.g., chloride, bromide, iodide), methanesulfonyl (mesyl), p-toluenesulfonyl (tosyl), trifluoromethylsulfonyl (triflate), and trifluoromethylsulfonate.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see T. W. Greene, Protective Groups in Organic Synthesis, John Wiley & Sons, New York, 1991, or a later edition.

### III. Methods

Provided herein are methods of treating a B-cell proliferative disorder (such as diffuse large B-cell lymphoma (DLBCL), e.g., relapsed/refractory DLBCL) in an individual (a human individual) in need thereof comprising administering to the individual an effective amount of: (a) an immunoconjugate comprising an antibody which binds CD79b linked to a cytotoxic agent, and (b) at least one additional therapeutic agent. In some embodiments, the at least one additional therapeutic agent is a chemotherapeutic agent. In some embodiments, the at least one additional therapeutic agent is cytotoxic agent. In some embodiments, the at least one additional therapeutic agent is an immunomodulatory agent. In some embodiments, the at least one additional therapeutic agent is an anti-CD20 agent, such as an anti-CD20 antibody.

In some embodiments, the methods comprise administering to the individual an effective amount of: (a) an immunoconjugate comprising an anti-CD79b antibody linked to a cytotoxic agent (i.e., anti-CD79b immunoconjugate), (b) an immunomodulatory agent, and (c) an anti-CD20 antibody.

Also provided herein are methods of treating diffuse large B-cell lymphoma (DLBCL, e.g., relapsed/refractory DLBCL) in an individual (a human individual) in need thereof comprising administering to the individual an effective amount of: (a) an immunoconjugate comprising the formula:

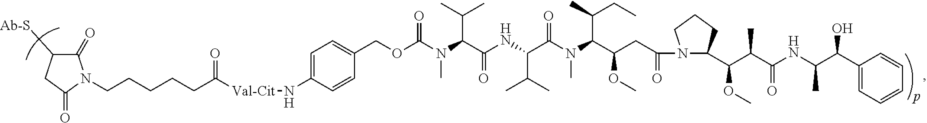

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8; (b) an immunomodulatory agent, and (c) an anti-CD20 antibody.

In some embodiments, the immunoconjugate comprises an anti-CD79b antibody that comprises a heavy chain variable domain (VH) comprising the amino acid sequence of SEQ ID NO: 19 and a light chain variable domain (VL) comprising the amino acid sequence of SEQ ID NO: 20. In some embodiments, the immunoconjugate comprises an anti-CD79b antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 38. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 35. In some embodiments, p is between 2 and 7, between 2 and 6, between 2 and 5, between 3 and 5, or between 3 and 4. In some embodiments, p is 3.4. In some embodiments, p is 3.5. In some embodiments, the anti-CD79b immunoconjugate is huMA79bv28-MC-vc-PAB-MMAE. In some embodiments, the immunoconjugate is polatuzumab vedotin (CAS Registry Number 1313206-42-6).

In some embodiments, the immunomodulatory agent is lenalidomide.

In some embodiments, the anti-CD20 antibody is a humanized B-Ly1 antibody. In some embodiments, the humanized B-Ly1 antibody is obinutuzumab. In some embodiments, the anti-CD20 antibody is rituximab. In some embodiments, the anti-CD20 antibody is ofatumumab, ublituximab, and/or ibritumomab tiuxetan.

In some embodiments, treatment of the individual, e.g., the human, according to any of the methods of the disclosure results in a response of at least stable disease (SD) (such as at least SD, at least partial response (PR), or a complete response/complete remission (CR)) during or after treatment (e.g., during or after a treatment regimen described herein). In some embodiments, treatment of the individual, e.g., the human, according to any of the methods of the disclosure results in an objective response, a best overall response, a best complete response, best partial response, or a complete response during or after treatment (e.g., during or after a treatment regimen described herein). Additional details regarding objective response, best overall response, best complete response, best partial response, complete response and other therapeutic responses are provided herein below.

A. Dosing and Administration

Anti-CD79b immunoconjugates and additional therapeutic agents (e.g., an immunomodulatory agent and an anti-CD20 agent) provided herein for use in any of the therapeutic methods described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immunoconjugate need not be, but is optionally formulated with one or more agents currently used to prevent or treat the disorder in question. The amount of the anti-CD79b immunoconjugate and the additional therapeutic agents (e.g., an immunomodulatory agent and an anti-CD20 agent), and the timing of co-administration will depend on the type (species, gender, age, weight, etc.) and condition of the patient being treated and the severity of the disease or condition being treated. The anti-CD79b immunoconjugate and the additional therapeutic agents (e.g., an immunomodulatory agent and an anti-CD20 agent) are suitably co-administered to the patient at one time or over a series of treatments, e.g., according to any of the treatment regimens described below.

In some embodiments, the dosage of the anti-CD79b immunoconjugate (such as huMA79bv28-MC-vc-PAB-MMAE or polatuzumab vedotin) is between about any of 1.4-5 mg/kg, 1.4-4 mg/kg, 1.4-3.2 mg/kg, 1.4-2.4 mg/kg, or 1.4-1.8 mg/kg. In some embodiments of any of the methods, the dosage of anti-CD79 immunoconjugate is about any of 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, and/or 4.8 mg/kg. In some embodiments, the dosage of anti-CD79b immunoconjugate is about 1.4 mg/kg. In some embodiments, the dosage of anti-CD79b immunoconjugate is about 1.8 mg/kg. In some embodiments, the dosage of anti-CD79b immunoconjugate is about 2.4 mg/kg. In some embodiments, the dosage of anti-CD79b immunoconjugate is about 3.2 mg/kg. In some embodiments, the dosage of anti-CD79b immunoconjugate is about 3.6 mg/kg. In some embodiments of any of the methods, the anti-CD79b immunoconjugate is administered q3w (i.e., once every 3 weeks). In some embodiments of any of the methods, the anti-CD79b immunoconjugate is administered q4w (i.e., once every 4 weeks). In some embodiments of any of the methods, the anti-CD79b immunoconjugate is administered once per month. In some embodiments of any of the methods, a month is 28 days. In some embodiments of any of the methods, the anti-CD79b immunoconjugate is administered once every 28 days. In some embodiments, the anti-CD79b immunoconjugate is administered via intravenous infusion. In some embodiments, the dosage administered via infusion is in the range of about 1 mg to about 1,500 mg per dose. Alternatively, the dosage range is of about 1 mg to about 1,500 mg, about 1 mg to about 1,000 mg, about 400 mg to about 1200 mg, about 600 mg to about 1000 mg, about 10 mg to about 500 mg, about 10 mg to about 300 mg, about 10 mg to about 200 mg, and about 1 mg to about 200 mg. In some embodiments, the dosage administered via infusion is in the range of about 1 μg/m² to about 10,000 μg/m² per dose. Alternatively, the dosage range is of about 1 μg/m² to about 1000 μg/m², about 1 μg/m² to about 800 μg/m², about 1 μg/m² to about 600 μg/m², about 1 μg/m² to about 400 μg/m², about 10 μg/m² to about 500 μg/m², about 10 μg/m² to about 300 μg/m², about 10 μg/m² to about 200 μg/m², and about 1 μg/m² to about 200 μg/m². The dose may be administered once per day, once per week, multiple times per week, but less than once per day, multiple times per month but less than once per day, multiple times per month but less than once per week, once per month, once every 28 days, or intermittently to relieve or alleviate symptoms of the disease. In some embodiments, the dosage of the immunoconjugate is 1.8 mg/kg, administered on day 1 of each 28-day cycle, or on day 1 of every month, wherein a month is 28 days. Administration may continue at any of the disclosed intervals until remission of the tumor or symptoms of the B-cell proliferative disorder being treated. Administration may continue after remission or relief of symptoms is achieved where such remission or relief is prolonged by such continued administration.

In some embodiments, the dosage of the anti-CD20 agent (e.g., an anti-CD20 antibody, such as rituximab or obinutuzumab) is between about 300-1600 mg/m² and/or 300-2000 mg. In some embodiments, the dosage of the anti-CD20 antibody is about any of 300, 375, 600, 1000, or 1250 mg/m² and/or 300, 1000, or 2000 mg. In some embodiments, the anti-CD20 antibody is rituximab and the dosage administered is 375 mg/m². In some embodiments, the anti-CD20 antibody is obinutuzumab and the dosage administered is 1000 mg. In some embodiments, the anti-CD20 antibody is administered q3w (i.e., every 3 weeks). In some embodiments, the anti-CD20 antibody is administered q4w (i.e., once every 4 weeks). In some embodiments, the anti-CD20 antibody is administered once per month. In some embodiments, a month is 28 days. In some embodiments, the anti-CD20 antibody is administered once every 28 days. In some embodiments, the dosage of an afucosylated anti-CD20 antibody (preferably the afucosylated humanized B-Ly1 antibody) may be 800 to 1600 mg (in one embodiment 800 to 1200 mg, such as 1000 mg) on days 1, 8, 15 of a 3- to 6-week dosage cycle and then in a dosage of 400 to 1200 mg (in one embodiment 800 to 1200 mg) on day 1 of up to nine 3- to 4-week dosage cycles. In some embodiments, the dose is a flat 1000 mg dose in a three-weeks-dosage schedule, with the possibility of an additional cycle of a flat dose of 1000 mg in the second week. In some embodiments, the dosage of rituximab is 375 mg/m², administered on day 1 of each month, or on day 1 of every two months. In some embodiments, the dosage of rituximab is 375 mg/m², administered on day 1 of each 28-day cycle, or on day 1 of every two months, wherein a month is 28 days. In some embodiments, the anti-CD20 antibody is administered via intravenous infusion.

In some embodiments, the dosage of the immunomodulatory agent, e.g., lenalidomide, is between about 5 mg and about 10 mg, between about 10 mg and about 15 mg, or between about 15 mg and about 20 mg. In some embodiments, the dosage of the immunomodulatory agent, e.g., lenalidomide, is between about 10 mg and about 20 mg. In some embodiments, the dosage of the immunomodulatory agent, e.g., lenalidomide, is about 5 mg, about 10 mg, about 15 mg, or about 20 mg. In some embodiments, the dosage of the immunomodulatory agent, e.g., lenalidomide, is about 5 mg. In some embodiments, the dosage of the immunomodulatory agent, e.g., lenalidomide, is about 10 mg. In some embodiments, the dosage of the immunomodulatory agent, e.g., lenalidomide, is about 15 mg. In some embodiments, the dosage of the immunomodulatory agent, e.g., lenalidomide, is about 20 mg. In some embodiments, the immunomodulatory agent, e.g., lenalidomide, is administered orally, e.g., in the form of capsules (e.g., capsules comprising 2.5 mg, 5 mg, 10 mg, 15 mg, 20 mg, or 25 mg of the immunomodulatory agent). In some embodiments, the immunomodulatory agent, e.g., lenalidomide, is administered daily (e.g., once per day). In some embodiments, the immunomodulatory agent, e.g., lenalidomide, may be administered daily (e.g., once per day) at a dose of about 5 mg, about 10 mg, about 15 mg, or about 20 mg on days 1-21 of each 28-day cycle of a treatment regimen, e.g., a treatment regimen described herein. In some embodiments, the immunomodulatory agent, e.g., lenalidomide, may be administered daily (e.g., once per day) at a dose of about 5 mg, about 10 mg, about 15 mg, or about 20 mg on days 1-21 of each month during a treatment regimen, e.g., a treatment regimen described herein.

An exemplary dosing regimen for the combination therapy of anti-CD79b immunoconjugates (such as huMA79bv28-MC-vc-PAB-MMAE or polatuzumab vedotin) and one or more additional therapeutic agents includes the anti-CD79 immunoconjugate (such as huMA79bv28-MC-vc-PAB-MMAE or polatuzumab vedotin) administered at a dose of about 1.4-5 mg/kg q4w, rituximab at a dose of about 375 mg/m² q4w, and immunomodulatory agent (e.g., lenalidomide) at a dose of about 10-20 mg on Days 1-21 of each 28-day cycle (e.g., each of days 1-21 q4w). Another exemplary dosing regimen for the combination therapy of anti-CD79b immunoconjugates (such as huMA79bv28-MC-vc-PAB-MMAE or polatuzumab vedotin) and one or more additional therapeutic agents includes anti-CD79b immunoconjugate (such as huMA79bv28-MC-vc-PAB-MMAE or polatuzumab vedotin) administered at a dose of about 1.4-5 mg/kg once every 28 days (e.g., on day 1 of each 28-day cycle), rituximab at a dose of about 375 mg/m² once every 28 days (e.g., on day 1 of each 28-day cycle), and immunomodulatory agent (e.g., lenalidomide) at a dose of about 10-20 mg on Days 1-21 of each 28-day cycle. In some embodiments, the anti-CD79b immunoconjugate is administered at a dose of about any of 1.4 mg/kg, 1.8 mg/kg, 2.0 mg/kg, 2.2 mg/kg, 2.4 mg/kg, 3.2 mg/kg, or 4.0 mg/kg. In some embodiments, the anti-CD79b immunoconjugate is administered at a dose of about 1.4 mg/kg. In some embodiments, the anti-CD79b immunoconjugate is administered at a dose of about 1.8 mg/kg. In some embodiments, the anti-CD79b immunoconjugate is administered at a dose of about 2.4 mg/kg. In some embodiments, the immunomodulatory agent (e.g., lenalidomide) is administered at a dose of about 10 mg. In some embodiments, the immunomodulatory agent (e.g., lenalidomide) is administered at a dose of about 15 mg. In some embodiments, the immunomodulatory agent (e.g., lenalidomide) is administered at a dose of about 20 mg.

Another exemplary dosing regimen for the combination therapy of anti-CD79b immunoconjugates (such as huMA79bv28-MC-vc-PAB-MMAE or polatuzumab vedotin) and one or more additional therapeutic agents includes the anti-CD79 immunoconjugate (such as huMA79bv28-MC-vc-PAB-MMAE or polatuzumab vedotin) administered at a dose of about 1.4-5 mg/kg q4w, obinutuzumab at a dose of about 1000 mg q4w, and immunomodulatory agent (e.g., lenalidomide) at a dose of about 10-20 mg on Days 1-21 of each 28-day cycle (e.g., each of days 1-21 q4w). Another exemplary dosing regimen for the combination therapy of anti-CD79b immunoconjugates (such as huMA79bv28-MC-vc-PAB-MMAE or polatuzumab vedotin) and one or more additional therapeutic agents includes anti-CD79 immunoconjugate (such as huMA79bv28-MC-vc-PAB-MMAE or polatuzumab vedotin) administered at a dose of about 1.4-5 mg/kg once every 28 days (e.g., on day 1 of each 28-day cycle), obinutuzumab at a dose of about 1000 mg once every 28 days (e.g., on day 1 of each 28-day cycle) or on days 1, 8, and 15 of each 28-day cycle, and immunomodulatory agent (e.g., lenalidomide) at a dose of about 10-20 mg on Days 1-21 of each 28-day cycle. In some embodiments, the anti-CD79 immunoconjugate is administered at a dose of about any of 1.4 mg/kg, 1.8 mg/kg, 2.0 mg/kg, 2.2 mg/kg, 2.4 mg/kg, 3.2 mg/kg, or 4.0 mg/kg. In some embodiments, the anti-CD79b immunoconjugate is administered at a dose of about 1.4 mg/kg. In some embodiments, the anti-CD79b immunoconjugate is administered at a dose of about 1.8 mg/kg. In some embodiments, the anti-CD79b immunoconjugate is administered at a dose of about 2.4 mg/kg. In some embodiments, the immunomodulatory agent (e.g., lenalidomide) is administered at a dose of about 10 mg. In some embodiments, the immunomodulatory agent (e.g., lenalidomide) is administered at a dose of about 15 mg. In some embodiments, the immunomodulatory agent (e.g., lenalidomide) is administered at a dose of about 20 mg.

The terms “co-administration,” “co-administering,” “combination,” or “in combination,” with respect to administration of two or more therapeutic agents, such as the anti-CD79b immunoconjugate and the at least one additional therapeutic agent (e.g., an immunomodulatory agent and an anti-CD20 agent), refer to the administration of the two or more therapeutic agents as two (or more) separate formulations, or as one single formulation comprising the two or more therapeutic agents. Where separate formulations are used, the co-administration can be simultaneous (i.e., at the same time) or sequential in any order, wherein preferably there is a time period while all active agents simultaneously exert their biological activities. In some embodiments, the two or more therapeutic agents are co-administered either simultaneously or sequentially. In some embodiments, when all therapeutic agents are co-administered sequentially, the dose of each agent is administered either on the same day in two or more separate administrations, or one of the agents is administered on day 1, the other agent(s) are co-administered on subsequent days, e.g., according to any of the treatment regimens described herein.

An immunoconjugate provided herein (and any additional therapeutic agents, e.g., an immunomodulatory agent and an anti-CD20 agent) for use in any of the therapeutic methods described herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including, but not limited to, single or multiple administrations over various time-points, bolus administration, and pulse infusion are contemplated herein. The anti-CD79b immunoconjugate (e.g., huMA79bv28-MC-vc-PAB-MMAE or polatuzumab vedotin), the immunomodulatory agent (such as lenalidomide) and the anti-CD20 antibody (such as obinutuzumab or rituximab) may be administered by the same route of administration or by different routes of administration. In some embodiments, the anti-CD79b immunoconjugate is administered intravenously, intramuscularly, subcutaneously, topically, orally, transdermally, intraperitoneally, intraorbitally, by implantation, by inhalation, intrathecally, intraventricularly, or intranasally. In some embodiments, the immunomodulatory agent (such as lenalidomide) is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD20 antibody (such as obinutuzumab or rituximab) is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD79b immunoconjugate and the anti-CD20 antibody (such as obinutuzumab or rituximab) are each administered via intravenous infusion, and the immunomodulatory agent (such as lenalidomide) is administered orally. An effective amount of the anti-CD79b immunoconjugate, the immunomodulatory agent (such as lenalidomide) and the anti-CD20 antibody (such as obinutuzumab or rituximab) may be administered for prevention or treatment of a disease, e.g., R/R DLBCL.

(i) Induction Phases

In some embodiments, the anti-CD79b immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab) are administered during an induction phase. An “induction phase” refers to a phase of treatment wherein the anti-CD79b immunoconjugate (such as huMA79bv28-MC-vc-PAB-MMAE or polatuzumab vedotin) is administered to an individual, e.g., a human.

In some embodiments, during the induction phase, the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab) are administered in 28-day cycles. In some embodiments, the induction phase comprises less than one complete 28-day cycle. In some embodiments, the induction phase comprises between one and six (e.g., any of 1, 2, 3, 4, 5, or 6) 28-day cycles. In some embodiments, the induction phase comprises at least six 28-day cycles.

In some embodiments, during the induction phase, the immunoconjugate (such as huMA79bv28-MC-vc-PAB-MMAE or polatuzumab vedotin) is administered intravenously at a dose of about 1.4 mg/kg on Day 1 of the first 28 day cycle, the immunomodulatory agent (e.g., lenalidomide) is administered orally at a dose of about 10 mg on each of Days 1-21 of the first 28 day cycle, and the anti-CD20 antibody is obinutuzumab, and the obinutuzumab is administered intravenously at a dose of about 1000 mg on each of Days 1, 8, and 15 of the first 28 day cycle; and the immunoconjugate (such as huMA79bv28-MC-vc-PAB-MMAE or polatuzumab vedotin) is administered intravenously at a dose of about 1.4 mg/kg on Day 1 of each of the second, third, fourth, fifth, and sixth 28-day cycles, the immunomodulatory agent (e.g., lenalidomide) is administered orally at a dose of about 10 mg on each of Days 1-21 of each of the second, third, fourth, fifth, and sixth 28-day cycles, and the obinutuzumab is administered intravenously at a dose of about 1000 mg on Day 1 of each of the second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.4 mg/kg on Day 1 of the first 28 day cycle, the immunomodulatory agent (e.g., lenalidomide) is administered orally at a dose of about 15 mg on each of Days 1-21 of the first 28 day cycle, and the anti-CD20 antibody is obinutuzumab, and the obinutuzumab is administered intravenously at a dose of about 1000 mg on each of Days 1, 8, and 15 of the first 28 day cycle; and the immunoconjugate (such as huMA79bv28-MC-vc-PAB-MMAE or polatuzumab vedotin) is administered intravenously at a dose of about 1.4 mg/kg on Day 1 of each of the second, third, fourth, fifth, and sixth 28-day cycles, the immunomodulatory agent (e.g., lenalidomide) is administered orally at a dose of about 15 mg on each of Days 1-21 of each of the second, third, fourth, fifth, and sixth 28-day cycles, and the obinutuzumab is administered intravenously at a dose of about 1000 mg on Day 1 of each of the second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.4 mg/kg on Day 1 of the first 28 day cycle, the immunomodulatory agent (e.g., lenalidomide) is administered orally at a dose of about 20 mg on each of Days 1-21 of the first 28 day cycle, and the anti-CD20 antibody is obinutuzumab, and the obinutuzumab is administered intravenously at a dose of about 1000 mg on each of Days 1, 8, and 15 of the first 28 day cycle; and the immunoconjugate (such as huMA79bv28-MC-vc-PAB-MMAE or polatuzumab vedotin) is administered intravenously at a dose of about 1.4 mg/kg on Day 1 of each of the second, third, fourth, fifth, and sixth 28-day cycles, the immunomodulatory agent (e.g., lenalidomide) is administered orally at a dose of about 20 mg on each of Days 1-21 of each of the second, third, fourth, fifth, and sixth 28-day cycles, and the obinutuzumab is administered intravenously at a dose of about 1000 mg on Day 1 of each of the second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.8 mg/kg on Day 1 of the first 28 day cycle, the immunomodulatory agent (e.g., lenalidomide) is administered orally at a dose of about 10 mg on each of Days 1-21 of the first 28 day cycle, and the anti-CD20 antibody is obinutuzumab, and the obinutuzumab is administered intravenously at a dose of about 1000 mg on each of Days 1, 8, and 15 of the first 28 day cycle; and the immunoconjugate (such as huMA79bv28-MC-vc-PAB-MMAE or polatuzumab vedotin) is administered intravenously at a dose of about 1.8 mg/kg on Day 1 of each of the second, third, fourth, fifth, and sixth 28-day cycles, the immunomodulatory agent (e.g., lenalidomide) is administered orally at a dose of about 10 mg on each of Days 1-21 of each of the second, third, fourth, fifth, and sixth 28-day cycles, and the obinutuzumab is administered intravenously at a dose of about 1000 mg on Day 1 of each of the second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.8 mg/kg on Day 1 of the first 28 day cycle, the immunomodulatory agent (e.g., lenalidomide) is administered orally at a dose of about 15 mg on each of Days 1-21 of the first 28 day cycle, and the anti-CD20 antibody is obinutuzumab, and the obinutuzumab is administered intravenously at a dose of about 1000 mg on each of Days 1, 8, and 15 of the first 28 day cycle; and the immunoconjugate (such as huMA79bv28-MC-vc-PAB-MMAE or polatuzumab vedotin) is administered intravenously at a dose of about 1.8 mg/kg on Day 1 of each of the second, third, fourth, fifth, and sixth 28-day cycles, the immunomodulatory agent (e.g., lenalidomide) is administered orally at a dose of about 15 mg on each of Days 1-21 of each of the second, third, fourth, fifth, and sixth 28-day cycles, and the obinutuzumab is administered intravenously at a dose of about 1000 mg on Day 1 of each of the second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.8 mg/kg on Day 1 of the first 28 day cycle, the immunomodulatory agent (e.g., lenalidomide) is administered orally at a dose of about 20 mg on each of Days 1-21 of the first 28 day cycle, and the anti-CD20 antibody is obinutuzumab, and the obinutuzumab is administered intravenously at a dose of about 1000 mg on each of Days 1, 8, and 15 of the first 28 day cycle; and the immunoconjugate (such as huMA79bv28-MC-vc-PAB-MMAE or polatuzumab vedotin) is administered intravenously at a dose of about 1.8 mg/kg on Day 1 of each of the second, third, fourth, fifth, and sixth 28-day cycles, the immunomodulatory agent (e.g., lenalidomide) is administered orally at a dose of about 20 mg on each of Days 1-21 of each of the second, third, fourth, fifth, and sixth 28-day cycles, and the obinutuzumab is administered intravenously at a dose of about 1000 mg on Day 1 of each of the second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.4 mg/kg on Day 1 of each 28-day cycle, the immunomodulatory agent (e.g., lenalidomide) is administered orally at a dose of about 10 mg on each of Days 1-21 of each 28-day cycle, and the anti-CD20 antibody is rituximab, and the rituximab is administered intravenously at a dose of about 375 mg/m² on Day 1 of each 28-day cycle.

In some embodiments, during the induction phase, the immunoconjugate (such as huMA79bv28-MC-vc-PAB-MMAE or polatuzumab vedotin) is administered intravenously at a dose of about 1.4 mg/kg on Day 1 of each 28-day cycle, the immunomodulatory agent (e.g., lenalidomide) is administered orally at a dose of about 15 mg on each of Days 1-21 of each 28-day cycle, and the anti-CD20 antibody is rituximab, and the rituximab is administered intravenously at a dose of about 375 mg/m² on Day 1 of each 28-day cycle.

In some embodiments, during the induction phase, the immunoconjugate (such as huMA79bv28-MC-vc-PAB-MMAE or polatuzumab vedotin) is administered intravenously at a dose of about 1.4 mg/kg on Day 1 of each 28-day cycle, the immunomodulatory agent (e.g., lenalidomide) is administered orally at a dose of about 20 mg on each of Days 1-21 of each 28-day cycle, and the anti-CD20 antibody is rituximab, and the rituximab is administered intravenously at a dose of about 375 mg/m² on Day 1 of each 28-day cycle.

In some embodiments, during the induction phase, the immunoconjugate (such as huMA79bv28-MC-vc-PAB-MMAE or polatuzumab vedotin) is administered intravenously at a dose of about 1.8 mg/kg on Day 1 of each 28-day cycle, the immunomodulatory agent (e.g., lenalidomide) is administered orally at a dose of about 10 mg on each of Days 1-21 of each 28-day cycle, and the anti-CD20 antibody is rituximab, and the rituximab is administered intravenously at a dose of about 375 mg/m² on Day 1 of each 28-day cycle.

In some embodiments, during the induction phase, the immunoconjugate (such as huMA79bv28-MC-vc-PAB-MMAE or polatuzumab vedotin) is administered intravenously at a dose of about 1.8 mg/kg on Day 1 of each 28-day cycle, the immunomodulatory agent (e.g., lenalidomide) is administered orally at a dose of about 15 mg on each of Days 1-21 of each 28-day cycle, and the anti-CD20 antibody is rituximab, and the rituximab is administered intravenously at a dose of about 375 mg/m² on Day 1 of each 28-day cycle.

In some embodiments, during the induction phase, the immunoconjugate (such as huMA79bv28-MC-vc-PAB-MMAE or polatuzumab vedotin) is administered intravenously at a dose of about 1.8 mg/kg on Day 1 of each 28-day cycle, the immunomodulatory agent (e.g., lenalidomide) is administered orally at a dose of about 20 mg on each of Days 1-21 of each 28-day cycle, and the anti-CD20 antibody is rituximab, and the rituximab is administered intravenously at a dose of about 375 mg/m² on Day 1 of each 28-day cycle.

In some embodiments, the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab) are administered for at least one 28-day cycle. In some embodiments, the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab) are administered for 1, 2, 3, 4, 5, 6, or more 28-day cycles. In some embodiments, the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab) are administered for up to six 28-day cycles. In some embodiments, the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab) are administered for six 28-day cycles.

In some embodiments, during the induction phase, the immunoconjugate (such as huMA79bv28-MC-vc-PAB-MMAE or polatuzumab vedotin) is administered intravenously at a dose of about 1.4 mg/kg on Day 1 of each of the first, second, third, fourth, fifth, and sixth 28-day cycles, the immunomodulatory agent (e.g., lenalidomide) is administered orally at a dose of about 10 mg on each of Days 1-21 of each of the first, second, third, fourth, fifth, and sixth 28-day cycles, and the anti-CD20 antibody is rituximab, and the rituximab is administered intravenously at a dose of about 375 mg/m² on Day 1 of each of the first, second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.4 mg/kg on Day 1 of each of the first, second, third, fourth, fifth, and sixth 28-day cycles, the immunomodulatory agent (e.g., lenalidomide) is administered orally at a dose of about 15 mg on each of Days 1-21 of each of the first, second, third, fourth, fifth, and sixth 28-day cycles, and the anti-CD20 antibody is rituximab, and the rituximab is administered intravenously at a dose of about 375 mg/m² on Day 1 of each of the first, second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.4 mg/kg on Day 1 of each of the first, second, third, fourth, fifth, and sixth 28-day cycles, the immunomodulatory agent (e.g., lenalidomide) is administered orally at a dose of about 20 mg on each of Days 1-21 of each of the first, second, third, fourth, fifth, and sixth 28-day cycles, and the anti-CD20 antibody is rituximab, and the rituximab is administered intravenously at a dose of about 375 mg/m² on Day 1 of each of the first, second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.8 mg/kg on Day 1 of each of the first, second, third, fourth, fifth, and sixth 28-day cycles, the immunomodulatory agent (e.g., lenalidomide) is administered orally at a dose of about 10 mg on each of Days 1-21 of each of the first, second, third, fourth, fifth, and sixth 28-day cycles, and the anti-CD20 antibody is rituximab, and the rituximab is administered intravenously at a dose of about 375 mg/m² on Day 1 of each of the first, second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.8 mg/kg on Day 1 of each of the first, second, third, fourth, fifth, and sixth 28-day cycles, the immunomodulatory agent (e.g., lenalidomide) is administered orally at a dose of about 15 mg on each of Days 1-21 of each of the first, second, third, fourth, fifth, and sixth 28-day cycles, and the anti-CD20 antibody is rituximab, and the rituximab is administered intravenously at a dose of about 375 mg/m² on Day 1 of each of the first, second, third, fourth, fifth, and sixth 28-day cycles.

In some embodiments, during the induction phase, the immunoconjugate (such as huMA79bv28-MC-vc-PAB-MMAE or polatuzumab vedotin) is administered intravenously at a dose of about 1.8 mg/kg on Day 1 of each of the first, second, third, fourth, fifth, and sixth 28-day cycles, the immunomodulatory agent (e.g., lenalidomide) is administered orally at a dose of about 20 mg on each of Days 1-21 of each of the first, second, third, fourth, fifth, and sixth 28-day cycles, and the anti-CD20 antibody is rituximab, and the rituximab is administered intravenously at a dose of about 375 mg/m² on Day 1 of each of the first, second, third, fourth, fifth, and sixth 28-day cycles.

The dosing and administration schedules for exemplary induction phases are provided in Tables A-L below:

Tables A-L: Dosing and Administration Schedules for Exemplary Induction Phases

TABLE A

| Drugs | Cycle 1 (28 days) | Cycles 2-6 (28 days each) |
|---|---|---|
| Anti-CD79b | 1.4 mg/kg on Day 1 | 1.4 mg/kg on Day 1 |
| immunoconjugate | | |
| (polatuzumab vedotin) | | |
| Immunomodulatory | 10 mg on each of Days 1-21 | 10 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE B

| Drugs | Cycle 1 (28 days) | Cycles 2-6 (28 days each) |
|---|---|---|
| Anti-CD79b | 1.4 mg/kg on Day 1 | 1.4 mg/kg on Day 1 |
| immunoconjugate | | |
| (polatuzumab vedotin) | | |
| Immunomodulatory | 15 mg on each of Days 1-21 | 15 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE C

| Drugs | Cycle 1 (28 days) | Cycles 2-6 (28 days each) |
|---|---|---|
| Anti-CD79b | 1.4 mg/kg on Day 1 | 1.4 mg/kg on Day 1 |
| immunoconjugate | | |
| (polatuzumab vedotin) | | |
| Immunomodulatory | 20 mg on each of Days 1-21 | 20 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE D

| Drugs | Cycle 1 (28 days) | Cycles 2-6 (28 days each) |
|---|---|---|
| Anti-CD79b | 1.8 mg/kg on Day 1 | 1.8 mg/kg on Day 1 |
| immunoconjugate | | |
| (polatuzumab vedotin) | | |
| Immunomodulatory | 10 mg on each of Days 1-21 | 10 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE E

| Drugs | Cycle 1 (28 days) | Cycles 2-6 (28 days each) |
|---|---|---|
| Anti-CD79b | 1.8 mg/kg on Day 1 | 1.8 mg/kg on Day 1 |
| immunoconjugate | | |
| (polatuzumab vedotin) | | |
| Immunomodulatory | 15 mg on each of Days 1-21 | 15 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE F

| Drugs | Cycle 1 (28 days) | Cycles 2-6 (28 days each) |
|---|---|---|
| Anti-CD79b | 1.8 mg/kg on Day 1 | 1.8 mg/kg on Day 1 |
| immunoconjugate | | |
| (polatuzumab vedotin) | | |
| Immunomodulatory | 20 mg on each of Days 1-21 | 20 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE G

| Drugs | Cycles 1-6 (28 days each) |
|---|---|
| Anti-CD79b immunoconjugate | 1.4 mg/kg on Day 1 |
| (polatuzumab vedotin) | |
| Immunomodulatory Agent | 10 mg on each of Days 1-21 |
| (lenalidomide) | |
| Anti-CD20 Antibody | 375 mg/m² on Day 1 |
| (rituximab) | |

TABLE H

| Drugs | Cycles 1-6 (28 days each) |
|---|---|
| Anti-CD79b immunoconjugate | 1.4 mg/kg on Day 1 |
| (polatuzumab vedotin) | |
| Immunomodulatory Agent | 15 mg on each of Days 1-21 |
| (lenalidomide) | |
| Anti-CD20 Antibody | 375 mg/m² on Day 1 |
| (rituximab) | |

TABLE I

| Drugs | Cycles 1-6 (28 days each) |
|---|---|
| Anti-CD79b immunoconjugate | 1.4 mg/kg on Day 1 |
| (polatuzumab vedotin) | |
| Immunomodulatory Agent | 20 mg on each of Days 1-21 |
| (lenalidomide) | |
| Anti-CD20 Antibody | 375 mg/m² on Day 1 |
| (rituximab) | |

TABLE J

| Drugs | Cycles 1-6 (28 days each) |
|---|---|
| Anti-CD79b immunoconjugate | 1.8 mg/kg on Day 1 |
| (polatuzumab vedotin) | |
| Immunomodulatory Agent | 10 mg on each of Days 1-21 |
| (lenalidomide) | |
| Anti-CD20 Antibody | 375 mg/m² on Day 1 |
| (rituximab) | |

TABLE K

| Drugs | Cycles 1-6 (28 days each) |
|---|---|
| Anti-CD79b immunoconjugate | 1.8 mg/kg on Day 1 |
| (polatuzumab vedotin) | |
| Immunomodulatory Agent | 15 mg on each of Days 1-21 |
| (lenalidomide) | |
| Anti-CD20 Antibody | 375 mg/m² on Day 1 |
| (rituximab) | |

TABLE L

| Drugs | Cycles 1-6 (28 days each) |
|---|---|
| Anti-CD79b immunoconjugate | 1.8 mg/kg on Day 1 |
| (polatuzumab vedotin) | |
| Immunomodulatory Agent | 20 mg on each of Days 1-21 |
| (lenalidomide) | |
| Anti-CD20 Antibody | 375 mg/m² on Day 1 |
| (rituximab) | |

In some embodiments, the anti-CD79b immunoconjugate (e.g., huMA79bv28-MC-vc-PAB-MMAE or polatuzumab vedotin), the immunomodulatory agent (e.g., lenalidomide), and the anti-CD20 antibody (e.g., obinutuzumab or rituximab) are administered sequentially during the induction phase, e.g., in the first, second, third, fourth, fifth, and sixth 28-day cycles. In some embodiments, the immunomodulatory agent (e.g., lenalidomide) is administered prior to the anti-CD20 antibody (e.g., obinutuzumab or rituximab), and the anti-CD20 antibody (e.g., obinutuzumab or rituximab) is administered prior to the immunoconjugate (e.g., huMA79bv28-MC-vc-PAB-MMAE or polatuzumab vedotin). In some embodiments, the immunomodulatory agent (e.g., lenalidomide) is administered prior to the anti-CD20 antibody (e.g., obinutuzumab or rituximab) and the anti-CD20 antibody (e.g., obinutuzumab or rituximab) is administered prior to the immunoconjugate (e.g., huMA79bv28-MC-vc-PAB-MMAE or polatuzumab vedotin) on Day 1 of each 28-day cycle.

(ii) Consolidation Phases

In some embodiments, the immunomodulatory agent (e.g., lenalidomide) and the anti-CD20 antibody (e.g., obinutuzumab or rituximab) are further administered during a consolidation phase after an induction phase described herein, e.g., after the last 28-day cycle of an induction phase described herein, for example, after the sixth 28-day cycle of an induction phase described herein. The “consolidation phase” refers to a treatment phase following an induction phase. In some embodiments, the consolidation phase begins immediately after the end of the induction phase. In some embodiments, the induction phase and the consolidation phase are separated by an interval of time. In some embodiments, the consolidation phase begins at least about 1, 2, 3, 4, 5, 6, 7, 8, 9, or 10 weeks after the end of the induction phase. In some embodiments, the consolidation phase begins about 7, about 8, or about 9 weeks after Day 1 of the final cycle of the induction phase. In some embodiments, the consolidation phase begins about 8 weeks after Day 1 of the final cycle of the induction phase. In some embodiments, the consolidation phase begins about 7, about 8, or about 9 weeks after Day 1 of the sixth 28-day cycle of the induction phase. In some embodiments, the consolidation phase begins about 8 weeks after Day 1 of the sixth 28-day cycle of the induction phase.

In some embodiments, the immunomodulatory agent (e.g., lenalidomide) is administered orally at a dose between about 10 mg and about 20 mg on each of Days 1-21 of each month during the consolidation phase, the anti-CD20 antibody is obinutuzumab, and the obinutuzumab is administered intravenously at a dose of about 1000 mg on Day 1 of every other month during the consolidation phase. In some embodiments, the immunomodulatory agent (e.g., lenalidomide) is administered orally at a dose of about 10 mg on each of Days 1-21 of each month during the consolidation phase, the anti-CD20 antibody is obinutuzumab, and the obinutuzumab is administered intravenously at a dose of about 1000 mg on Day 1 of every other month during the consolidation phase. In some embodiments, the immunomodulatory agent (e.g., lenalidomide) is administered orally at a dose of about 15 mg on each of Days 1-21 of each month during the consolidation phase, the anti-CD20 antibody is obinutuzumab, and the obinutuzumab is administered intravenously at a dose of about 1000 mg on Day 1 of every other month during the consolidation phase. In some embodiments, the immunomodulatory agent (e.g., lenalidomide) is administered orally at a dose of about 20 mg on each of Days 1-21 of each month during the consolidation phase, the anti-CD20 antibody is obinutuzumab, and the obinutuzumab is administered intravenously at a dose of about 1000 mg on Day 1 of every other month during the consolidation phase. In some embodiments, a month includes 28 days. In some embodiments, the immunomodulatory agent (e.g., lenalidomide) is administered for any of 1, 2, 3, 4, 5, 6, or more months during the consolidation phase. In some embodiments, the immunomodulatory agent (e.g., lenalidomide) is administered for up to 6 months during the consolidation phase. In some embodiments, the anti-CD20 antibody (e.g., obinutuzumab) is administered beginning with month 1 of the consolidation phase. In some embodiments, the anti-CD20 antibody (e.g., obinutuzumab) is administered on day 1 of each of months 1, 3, and 5 of the consolidation phase.

In some embodiments, the immunomodulatory agent (e.g., lenalidomide) is administered orally at a dose between about 10 mg and about 20 mg on each of Days 1-21 of each month during the consolidation phase, the anti-CD20 antibody is rituximab, and the rituximab is administered intravenously at a dose of about 375 mg/m² on Day 1 of every other month during the consolidation phase. In some embodiments, the immunomodulatory agent (e.g., lenalidomide) is administered orally at a dose of about 10 mg on each of Days 1-21 of each month during the consolidation phase, the anti-CD20 antibody is rituximab, and the rituximab is administered intravenously at a dose of about 375 mg/m² on Day 1 of every other month during the consolidation phase. In some embodiments, the immunomodulatory agent (e.g., lenalidomide) is administered orally at a dose of about 15 mg on each of Days 1-21 of each month during the consolidation phase, the anti-CD20 antibody is rituximab, and the rituximab is administered intravenously at a dose of about 375 mg/m² on Day 1 of every other month during the consolidation phase. In some embodiments, the immunomodulatory agent (e.g., lenalidomide) is administered orally at a dose of about 20 mg on each of Days 1-21 of each month during the consolidation phase, the anti-CD20 antibody is rituximab, and the rituximab is administered intravenously at a dose of about 375 mg/m² on Day 1 of every other month during the consolidation phase. In some embodiments, a month includes 28 days. In some embodiments, the immunomodulatory agent (e.g., lenalidomide) is administered for any of 1, 2, 3, 4, 5, 6, or more months during the consolidation phase. In some embodiments, the immunomodulatory agent (e.g., lenalidomide) is administered for up to 6 months during the consolidation phase. In some embodiments, the anti-CD20 antibody (e.g., rituximab) is administered beginning with month 1 of the consolidation phase. In some embodiments, the anti-CD20 antibody (e.g., rituximab) is administered on day 1 of each of months 1, 3, and 5 of the consolidation phase.

In some embodiments, the immunomodulatory agent (e.g., lenalidomide) and the anti-CD20 antibody (e.g., obinutuzumab or rituximab) are administered sequentially during the consolidation phase. In some embodiments, the immunomodulatory agent (e.g., lenalidomide) is administered prior to the anti-CD20 antibody (e.g., obinutuzumab or rituximab) during the consolidation phase. In some embodiments, the immunomodulatory agent (e.g., lenalidomide) is administered prior to the anti-CD20 antibody (e.g., obinutuzumab or rituximab) on Day 1 of each of the first, third, and fifth months during the consolidation phase.

The dosing and administration schedules for exemplary consolidation phases are provided in Tables M-R below:

Tables M-R: Dosing and Administration Schedules for Exemplary Consolidation Phases

TABLE M

| Drugs | Dose and Frequency of Administration |
|---|---|
| Immunomodulatory | 10 mg on each of Days 1-21 every month |
| Agent | |
| (lenalidomide) | |
| Anti-CD20 Antibody | 1000 mg on Day 1 of every other month |
| (obinutuzumab) | |

TABLE N

| Drugs | Dose and Frequency of Administration |
|---|---|
| Immunomodulatory | 15 mg on each of Days 1-21 every month |
| Agent | |
| (lenalidomide) | |
| Anti-CD20 Antibody | 1000 mg on Day 1 of every other month |
| (obinutuzumab) | |

TABLE O

| Drugs | Dose and Frequency of Administration |
|---|---|
| Immunomodulatory | 20 mg on each of Days 1-21 every month |
| Agent | |
| (lenalidomide) | |
| Anti-CD20 Antibody | 1000 mg on Day 1 of every other month |
| (obinutuzumab) | |

TABLE P

| Drugs | Dose and Frequency of Administration |
|---|---|
| Immunomodulatory | 10 mg on each of Days 1-21 every month |
| Agent | |
| (lenalidomide) | |
| Anti-CD20 Antibody | 375 mg/m² on Day 1 of every other month |
| (rituximab) | |

TABLE Q

| Drugs | Dose and Frequency of Administration |
|---|---|
| Immunomodulatory | 15 mg on each of Days 1-21 every month |
| Agent | |
| (lenalidomide) | |
| Anti-CD20 Antibody | 375 mg/m² on Day 1 of every other month |
| (rituximab) | |

TABLE R

| Drugs | Dose and Frequency of Administration |
|---|---|
| Immunomodulatory | 20 mg on each of Days 1-21 every month |
| Agent | |
| (lenalidomide) | |
| Anti-CD20 Antibody | 375 mg/m² on Day 1 of every other month |
| (rituximab) | |

B. Exemplary Treatment Regimens

Any one of the exemplary induction phases described herein or shown in Tables A-L may be followed by any one of the exemplary consolidation phases described herein or shown in Tables M-R.

In some embodiments, the methods for treating diffuse large B-cell lymphoma (DLBCL) in an individual, e.g., a human, in need thereof provided herein comprise administering (a) an immunoconjugate comprising the formula:

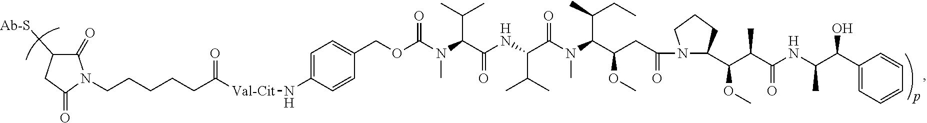

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) an immunomodulatory agent, and (c) an anti-CD20 antibody.

In some embodiments, the methods for treating diffuse large B-cell lymphoma (DLBCL) in an individual, e.g., a human, in need thereof comprise administering to the individual an effective amount of:
(a) an immunoconjugate comprising the formula:

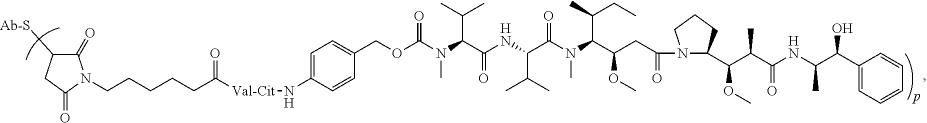

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) an immunomodulatory agent, and (c) an anti-CD20 antibody. In some embodiments, p is between 2 and 5. In some embodiments, p is between 3 and 4. In some embodiments, p is 3.4. In some embodiments, p is 3.5. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35.

In some embodiments, the methods for treating diffuse large B-cell lymphoma (DLBCL) in an individual, e.g., a human, in need thereof comprise administering to the individual an effective amount of: (a) an immunoconjugate comprising the formula:

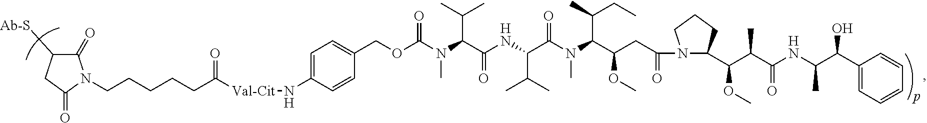

wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, (b) an immunomodulatory agent, and (c) an anti-CD20 antibody. In some embodiments, p is between 3 and 4. In some embodiments, p is 3.4. In some embodiments, p is 3.5. In some embodiments, the antibody comprises (i) a heavy chain comprising the amino acid sequence of SEQ ID NO: 36 and (ii) a light chain comprising the amino acid sequence of SEQ ID NO: 35.

In some embodiments, the immunoconjugate is administered at a dose of about 1.8 mg/kg, the immunomodulatory agent is administered at a dose between about 10 mg and about 20 mg, and the anti-CD20 antibody is rituximab administered at a dose of about 375 mg/m². In some embodiments, the immunoconjugate, the immunomodulatory agent, and the rituximab are administered during an induction phase in 28-day cycles, wherein: the immunoconjugate is administered intravenously at a dose of about 1.8 mg/kg on Day 1 of each 28-day cycle, the immunomodulatory agent is administered orally at a dose between about 10 mg and about 20 mg on each of Days 1-21 of each 28-day cycle, and the rituximab is administered intravenously at a dose of about 375 mg/m² on Day 1 of each 28-day cycle. In some embodiments, the immunomodulatory agent is administered at a dose of about 20 mg. In some embodiments, the induction phase comprises less than one complete 28-day cycle. In some embodiments, the induction phase comprises between one and six (e.g., any of 1, 2, 3, 4, 5, or 6) 28-day cycles. In some embodiments, the induction phase comprises at least six 28-day cycles. In some embodiments, the induction phase comprises six 28-day cycles. In some embodiments, the immunoconjugate, the immunomodulatory agent, and the rituximab are administered sequentially. In some embodiments, the immunomodulatory agent is administered prior to the rituximab and the rituximab is administered prior to the immunoconjugate on Day 1 of each 28-day cycle. In some embodiments, the immunomodulatory agent and the rituximab are further administered during a consolidation phase after the sixth 28-day cycle of the induction phase. In some embodiments, the immunomodulatory agent is administered orally at a dose of about 10 mg on each of Days 1-21 of each month during the consolidation phase, and the rituximab is administered intravenously at a dose of about 375 mg/m² on Day 1 of every other month during the consolidation phase. In some embodiments, the immunomodulatory agent is administered for a maximum of 6 months during the consolidation phase. In some embodiments, the rituximab is administered on Day 1 of each of the first, third, and fifth months during the consolidation phase. In some embodiments, the immunomodulatory agent and the rituximab are administered sequentially during the consolidation phase. In some embodiments, the immunomodulatory agent is administered prior to the rituximab on Day 1 of each of the first, third, and fifth months during the consolidation phase. In some embodiments, a month during the consolidation phase comprises 28 days. In some embodiments, the consolidation phase begins immediately after the end of the induction phase. In some embodiments, the induction phase and the consolidation phase are separated by an interval of time. In some embodiments, the consolidation phase begins at least about 1, 2, 3, 4, 5, 6, 7, 8, 9, or 10 weeks after the end of the induction phase. In some embodiments, the consolidation phase begins about 7, about 8, or about 9 weeks after Day 1 of the final cycle of the induction phase. In some embodiments, the consolidation phase begins about 8 weeks after Day 1 of the final cycle of the induction phase. In some embodiments, the consolidation phase begins about 7, about 8, or about 9 weeks after Day 1 of the sixth 28-day cycle of the induction phase. In some embodiments, the consolidation phase begins about 8 weeks after Day 1 of the sixth 28-day cycle of the induction phase.

In some embodiments, the immunoconjugate is polatuzumab vedotin. In some embodiments, the immunomodulatory agent is lenalidomide. In some embodiments, the anti-CD20 antibody is rituximab. In some embodiments, the polatuzumab vedotin is administered at a dose of about 1.8 mg/kg, the lenalidomide is administered at a dose between about 10 mg and about 20 mg, and the rituximab is administered at a dose of about 375 mg/m². In some embodiments, the lenalidomide is administered at a dose of about 20 mg. In some embodiments, the lenalidomide is administered at a dose of about 10 mg. In some embodiments, the polatuzumab vedotin, the lenalidomide, and the rituximab are administered during an induction phase in 28-day cycles, wherein: the polatuzumab vedotin is administered intravenously at a dose of about 1.8 mg/kg on Day 1 of each 28-day cycle, the lenalidomide is administered orally at a dose between about 10 mg and about 20 mg on each of Days 1-21 of each 28-day cycle, and the rituximab is administered intravenously at a dose of about 375 mg/m² on Day 1 of each 28-day cycle. In some embodiments, the lenalidomide is administered at a dose of about 20 mg. In some embodiments, the induction phase comprises less than one complete 28-day cycle. In some embodiments, the induction phase comprises between one and six (e.g., any of 1, 2, 3, 4, 5, or 6) 28-day cycles. In some embodiments, the induction phase comprises at least six 28-day cycles. In some embodiments, the induction phase comprises six 28-day cycles. In some embodiments, the polatuzumab vedotin, the lenalidomide, and the rituximab are administered sequentially. In some embodiments, the lenalidomide is administered prior to the rituximab and the rituximab is administered prior to the polatuzumab vedotin on Day 1 of each 28-day cycle. In some embodiments, the lenalidomide and the rituximab are further administered during a consolidation phase after the sixth 28-day cycle of the induction phase. In some embodiments, the lenalidomide is administered orally at a dose of about 10 mg on each of Days 1-21 of each month during the consolidation phase, and the rituximab is administered intravenously at a dose of about 375 mg/m² on Day 1 of every other month during the consolidation phase. In some embodiments, the lenalidomide is administered for a maximum of 6 months during the consolidation phase. In some embodiments, the rituximab is administered on Day 1 of each of the first, third, and fifth months during the consolidation phase. In some embodiments, the lenalidomide and the rituximab are administered sequentially during the consolidation phase. In some embodiments, the lenalidomide is administered prior to the rituximab on Day 1 of each of the first, third, and fifth months during the consolidation phase. In some embodiments, a month during the consolidation phase comprises 28 days. In some embodiments, the consolidation phase begins immediately after the end of the induction phase. In some embodiments, the induction phase and the consolidation phase are separated by an interval of time. In some embodiments, the consolidation phase begins at least about 1, 2, 3, 4, 5, 6, 7, 8, 9, or 10 weeks after the end of the induction phase. In some embodiments, the consolidation phase begins about 7, about 8, or about 9 weeks after Day 1 of the final cycle of the induction phase. In some embodiments, the consolidation phase begins about 8 weeks after Day 1 of the final cycle of the induction phase. In some embodiments, the consolidation phase begins about 7, about 8, or about 9 weeks after Day 1 of the sixth 28-day cycle of the induction phase. In some embodiments, the consolidation phase begins about 8 weeks after Day 1 of the sixth 28-day cycle of the induction phase.

In some embodiments, the methods for treating diffuse large B-cell lymphoma (DLBCL) in an individual, e.g., a human, in need thereof comprise administering to the individual an effective amount of: (a) polatuzumab vedotin; (b) lenalidomide; and (c) rituximab. In some embodiments, the polatuzumab vedotin, lenalidomide, and rituximab are administered during an induction phase, e.g., an induction phase described herein, in 28-day cycles. In some embodiments, the induction phase comprises less than one complete 28-day cycle. In some embodiments, the induction phase comprises between one and six (e.g., any of 1, 2, 3, 4, 5, or 6) 28-day cycles. In some embodiments, the induction phase comprises at least six 28-day cycles. In some embodiments, the induction phase comprises six 28-day cycles. In some embodiments, the polatuzumab vedotin is administered intravenously at a dose of about 1.8 mg/kg on Day 1 of each 28-day cycle, the lenalidomide is administered orally at a dose between about 10 mg and about 20 mg on each of Days 1-21 of each 28-day cycle, and the rituximab is administered intravenously at a dose of about 375 mg/m² on Day 1 of each 28-day cycle. In some embodiments, the lenalidomide is administered at a dose of about 20 mg. In some embodiments, the polatuzumab vedotin, the lenalidomide, and the rituximab are administered sequentially. In some embodiments, the lenalidomide is administered prior to the rituximab and the rituximab is administered prior to the polatuzumab vedotin on Day 1 of each 28-day cycle. In some embodiments, the induction phase is followed by a consolidation phase, wherein the lenalidomide is administered at a dose of about 10 mg and the rituximab is administered at a dose of about 375 mg/m² during the consolidation phase. In some embodiments, the lenalidomide is administered orally at a dose of about 10 mg on each of Days 1-21 of each month during the consolidation phase, and the rituximab is administered intravenously at a dose of about 375 mg/m² on Day 1 of every other month during the consolidation phase. In some embodiments, the lenalidomide is administered for a maximum of 6 months during the consolidation phase. In some embodiments, the rituximab is administered on Day 1 of each of the first, third, and fifth months during the consolidation phase. In some embodiments, the lenalidomide and the rituximab are administered sequentially during the consolidation phase. In some embodiments, the lenalidomide is administered prior to the rituximab on Day 1 of each of the first, third, and fifth months during the consolidation phase. In some embodiments, a month during the consolidation phase comprises 28 days. In some embodiments, the consolidation phase begins immediately after the end of the induction phase. In some embodiments, the induction phase and the consolidation phase are separated by an interval of time. In some embodiments, the consolidation phase begins at least about 1, 2, 3, 4, 5, 6, 7, 8, 9, or 10 weeks after the end of the induction phase. In some embodiments, the consolidation phase begins about 7, about 8, or about 9 weeks after Day 1 of the final cycle of the induction phase. In some embodiments, the consolidation phase begins about 8 weeks after Day 1 of the final cycle of the induction phase. In some embodiments, the consolidation phase begins about 7, about 8, or about 9 weeks after Day 1 of the sixth 28-day cycle of the induction phase. In some embodiments, the consolidation phase begins about 8 weeks after Day 1 of the sixth 28-day cycle of the induction phase.

C. Responses

In some embodiments, a human treated according to any of the methods described herein achieves at least stable disease (SD) (e.g., at least SD, at least partial response (PR) or a complete response (CR)) during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab). In some embodiments, a human treated according to any of the methods described herein achieves at least a partial response (PR) (e.g., at least PR or a complete response (CR)) during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab). In some embodiments, a human treated according to any of the methods described herein achieves a complete response (CR) during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab). In some embodiments, a human treated according to any of the methods described herein does not demonstrate disease progression within at least about 4 months after the start of treatment with the immunoconjugate (such as huMA79bv28-MC-vc-PAB-MMAE or polatuzumab vedotin), the immunomodulatory agent (e.g., lenalidomide) and the anti-CD20 antibody (e.g., obinutuzumab or rituximab). In some embodiments, a human treated according to any of the methods described herein achieves an improved response compared to a human treated with a treatment comprising a single agent, e.g., a treatment with only an immunoconjugate (such as huMA79bv28-MC-vc-PAB-MMAE or polatuzumab vedotin), a treatment with only an immunomodulatory agent (e.g., lenalidomide), or a treatment with only an anti-CD20 antibody (e.g., obinutuzumab or rituximab). In some embodiments, a human treated according to any of the methods described herein achieves an improved response compared to a human treated with a treatment comprising a double combination of an immunoconjugate (such as huMA79bv28-MC-vc-PAB-MMAE or polatuzumab vedotin) and an immunomodulatory agent (e.g., lenalidomide). In some embodiments, a human treated according to any of the methods described herein achieves an improved response compared to a human treated with a treatment comprising a double combination of an immunoconjugate (such as huMA79bv28-MC-vc-PAB-MMAE or polatuzumab vedotin) and an anti-CD20 antibody (e.g., obinutuzumab or rituximab). In some embodiments, a human treated according to any of the methods described herein achieves an improved response compared to a human treated with a treatment comprising a double combination of an immunomodulatory agent (e.g., lenalidomide) and an anti-CD20 antibody (e.g., obinutuzumab or rituximab).

In some embodiments, among a plurality of humans treated according to any of the methods described herein, at least about 25%, at least about 27%, at least about 29%, at least about 30%, at least about 40%, at least about 50%, at least about 60%, at least about 70%, at least about 80%, at least about 90%, or 100% of the humans achieve stable disease during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any of the methods described herein, at least about 25%, at least about 27%, at least about 29%, at least about 30%, at least about 40%, at least about 50%, at least about 60%, at least about 70%, at least about 80%, at least about 90%, or 100% of the humans achieve a partial response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any of the methods described herein, at least about 25%, at least about 27%, at least about 29%, at least about 30%, at least about 40%, at least about 50%, at least about 60%, at least about 70%, at least about 80%, at least about 90%, or 100% of the humans achieve a complete response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any of the methods described herein, at least about 70%, at least about 74%, at least about 80%, at least about 90%, or 100% of the humans achieve a best overall response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any of the methods described herein, at least about 30%, at least about 35%, at least about 39%, at least about 40%, at least about 50%, at least about 60%, at least about 70%, at least about 80%, at least about 90%, or 100% of the humans achieve an objective response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any of the methods described herein, at least about 30%, at least about 35%, at least about 40%, at least about 50%, at least about 60%, at least about 70%, at least about 80%, at least about 90%, or 100% of the humans achieve a best partial response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any of the methods described herein, at least about 30%, at least about 35%, at least about 40%, at least about 50%, at least about 60%, at least about 70%, at least about 80%, at least about 90%, or 100% of the humans achieve a best complete response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, the duration of the response (i.e., of the stable disease response, partial response, complete response, objective response, best overall response, best complete response, or best partial response) is at least about 4 months, at least about 5 months, at least about 6 months, at least about 7 months, at least about 8 months, at least about 9 months, or more.

In some embodiments, a human treated according to any of the methods described herein survives for at least about 4 months, at least about 5 months, at least about 6 months, at least about 7 months, at least about 8 months, at least about 9 months, at least about 10 months, or more, without disease progression, assessed from the start of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any of the methods described herein, the median progression-free survival (PFS) is at least about 4 months, at least about 5 months, at least about 6 months, at least about 7 months, at least about 8 months, at least about 9 months, or more.

In some embodiments, a human treated according to any of the methods described herein survives for at least about 7 months, at least about 8 months, at least about 9 months, at least about 10 months, at least about 11 months, or more, assessed from the start of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any of the methods described herein, the median overall survival is at least about 7 months, at least about 8 months, at least about 9 months, at least about 10 months, at least about 11 months, or more, assessed from the start of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab) according to any of the methods described herein does not result in tumor lysis syndrome in the human.

In some embodiments, treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab) according to any of the methods described herein does not result in a second malignancy in the human.

In some embodiments, a human treated according to an induction phase described herein achieves at least stable disease (SD) (e.g., at least SD, at least partial response (PR) or a complete response (CR)) during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab), e.g., during or after the induction phase, such as after less than one 28-day cycle, or after at least any of 1, 2, 3, 4, 5, 6, or more 28-day cycles. In some embodiments, a human treated according to an induction phase described herein achieves at least stable disease (SD) (e.g., at least SD, at least partial response (PR) or a complete response (CR)) after six 28-day cycles. In some embodiments, a human treated according to an induction phase described herein achieves at least partial response (PR) during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab), e.g., during or after the induction phase, such as after less than one 28-day cycle, or after at least any of 1, 2, 3, 4, 5, 6, or more 28-day cycles. In some embodiments, a human treated according to an induction phase described herein achieves at least partial response (PR) after six 28-day cycles. In some embodiments, a human treated according to an induction phase described herein achieves complete response (CR) during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab), e.g., during or after the induction phase, such as after less than one 28-day cycle, or after at least any of 1, 2, 3, 4, 5, 6, or more 28-day cycles. In some embodiments, a human treated according to an induction phase described herein achieves at least complete response (CR) after six 28-day cycles. In some embodiments, a human treated according to any induction phase described herein does not demonstrate disease progression within at least about 4 months after the start of treatment with the immunoconjugate (such as huMA79bv28-MC-vc-PAB-MMAE or polatuzumab vedotin), the immunomodulatory agent (e.g., lenalidomide) and the anti-CD20 antibody (e.g., obinutuzumab or rituximab). In some embodiments, a human treated according to any induction phase described herein achieves an improved response compared to a human treated with a treatment comprising a single agent, e.g., a treatment with only an immunoconjugate (such as huMA79bv28-MC-vc-PAB-MMAE or polatuzumab vedotin), a treatment with only an immunomodulatory agent (e.g., lenalidomide), or a treatment with only an anti-CD20 antibody (e.g., obinutuzumab or rituximab). In some embodiments, a human treated according to any induction phase described herein achieves an improved response compared to a human treated with a treatment comprising a double combination of an immunoconjugate (such as huMA79bv28-MC-vc-PAB-MMAE or polatuzumab vedotin) and an immunomodulatory agent (e.g., lenalidomide). In some embodiments, a human treated according to any induction phase described herein achieves an improved response compared to a human treated with a treatment comprising a double combination of an immunoconjugate (such as huMA79bv28-MC-vc-PAB-MMAE or polatuzumab vedotin) and an anti-CD20 antibody (e.g., obinutuzumab or rituximab). In some embodiments, a human treated according to any induction phase described herein achieves an improved response compared to a human treated with a treatment comprising a double combination of an immunomodulatory agent (e.g., lenalidomide) and an anti-CD20 antibody (e.g., obinutuzumab or rituximab).

In some embodiments, among a plurality of humans treated according to any induction phase described herein, at least about 25%, at least about 27%, at least about 29%, at least about 30%, at least about 40%, at least about 50%, at least about 60%, at least about 70%, at least about 80%, at least about 90%, or 100% of the humans achieve a stable disease during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab), e.g., during or after the induction phase, such as after less than one 28-day cycle, or after at least any of 1, 2, 3, 4, 5, 6, or more 28-day cycles. In some embodiments, among a plurality of humans treated according to any induction phase described herein, at least about 25%, at least about 27%, at least about 29%, at least about 30%, at least about 40%, at least about 50%, at least about 60%, at least about 70%, at least about 80%, at least about 90%, or 100% of the humans achieve a stable disease after six 28-day cycles of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any induction phase described herein, at least about 25%, at least about 27%, at least about 29%, at least about 30%, at least about 40%, at least about 50%, at least about 60%, at least about 70%, at least about 80%, at least about 90%, or 100% of the humans achieve a partial response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab), e.g., during or after the induction phase, such as after less than one 28-day cycle, or after at least any of 1, 2, 3, 4, 5, 6, or more 28-day cycles. In some embodiments, among a plurality of humans treated according to any induction phase described herein, at least about 25%, at least about 27%, at least about 29%, at least about 30%, at least about 40%, at least about 50%, at least about 60%, at least about 70%, at least about 80%, at least about 90%, or 100% of the humans achieve a partial response after six 28-day cycles of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any induction phase described herein, at least about 25%, at least about 27%, at least about 29%, at least about 30%, at least about 40%, at least about 50%, at least about 60%, at least about 70%, at least about 80%, at least about 90%, or 100% of the humans achieve a complete response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab), e.g., during or after the induction phase, such as after less than one 28-day cycle, or after at least any of 1, 2, 3, 4, 5, 6, or more 28-day cycles. In some embodiments, among a plurality of humans treated according to any induction phase described herein, at least about 25%, at least about 27%, at least about 29%, at least about 30%, at least about 40%, at least about 50%, at least about 60%, at least about 70%, at least about 80%, at least about 90%, or 100% of the humans achieve a complete response after six 28-day cycles of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any induction phase described herein, at least about 70%, at least about 74%, at least about 80%, at least about 90%, or 100% of the humans achieve a best overall response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab), e.g., during or after the induction phase, such as after less than one 28-day cycle, or after at least any of 1, 2, 3, 4, 5, 6, or more 28-day cycles. In some embodiments, among a plurality of humans treated according to any induction phase described herein, at least about 70%, at least about 74%, at least about 80%, at least about 90%, or 100% of the humans achieve a best overall response after six 28-day cycles of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any induction phase described herein, at least about 30%, at least about 35%, at least about 39%, at least about 40%, at least about 50%, at least about 60%, at least about 70%, at least about 80%, at least about 90%, or 100% of the humans achieve an objective response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab), e.g., during or after the induction phase, such as after less than one 28-day cycle, or after at least any of 1, 2, 3, 4, 5, 6, or more 28-day cycles. In some embodiments, among a plurality of humans treated according to any induction phase described herein, at least about 30%, at least about 35%, at least about 39%, at least about 40%, at least about 50%, at least about 60%, at least about 70%, at least about 80%, at least about 90%, or 100% of the humans achieve an objective response after six 28-day cycles of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any induction phase described herein, at least about 30%, at least about 35%, at least about 40%, at least about 50%, at least about 60%, at least about 70%, at least about 80%, at least about 90%, or 100% of the humans achieve a best partial response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab), e.g., during or after the induction phase, such as after less than one 28-day cycle, or after at least any of 1, 2, 3, 4, 5, 6, or more 28-day cycles. In some embodiments, among a plurality of humans treated according to any induction phase described herein, at least about 30%, at least about 35%, at least about 40%, at least about 50%, at least about 60%, at least about 70%, at least about 80%, at least about 90%, or 100% of the humans achieve a best partial response after six 28-day cycles of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any induction phase described herein, at least about 30%, at least about 35%, at least about 40%, at least about 50%, at least about 60%, at least about 70%, at least about 80%, at least about 90%, or 100% of the humans achieve a best complete response during or after treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab), e.g., during or after the induction phase, such as after less than one 28-day cycle, or after at least any of 1, 2, 3, 4, 5, 6, or more 28-day cycles. In some embodiments, among a plurality of humans treated according to any induction phase described herein, at least about 30%, at least about 35%, at least about 40%, at least about 50%, at least about 60%, at least about 70%, at least about 80%, at least about 90%, or 100% of the humans achieve a best complete response after six 28-day cycles of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, the duration of the response (i.e., of the stable disease response, partial response, complete response, objective response, best overall response, best complete response, or best partial response) is at least about 4 months, at least about 5 months, at least about 6 months, at least about 7 months, at least about 8 months, at least about 9 months, or more.

In some embodiments, a human treated according to any induction phase described herein survives for at least about 4 months, at least about 5 months, at least about 6 months, at least about 7 months, at least about 8 months, at least about 9 months, at least about 10 months, or more, without disease progression, assessed from the start of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any induction phase described herein, the median progression-free survival (PFS) is at least about 4 months, at least about 5 months, at least about 6 months, at least about 7 months, at least about 8 months, at least about 9 months, or more.

In some embodiments, a human treated according to any induction phase described herein survives for at least about 7 months, at least about 8 months, at least about 9 months, at least about 10 months, at least about 11 months, or more, assessed from the start of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, among a plurality of humans treated according to any induction phase described herein, the median overall survival is at least about 7 months, at least about 8 months, at least about 9 months, at least about 10 months, at least about 11 months, or more, assessed from the start of treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab).

In some embodiments, treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab) according to any induction phase described herein does not result in tumor lysis syndrome in the human.

In some embodiments, treatment with the immunoconjugate (such as huMA79bv28-MC-vc-PAB-MMAE or polatuzumab vedotin), the immunomodulatory agent (e.g., lenalidomide), and the anti-CD20 antibody (e.g., obinutuzumab or rituximab) according to any induction phase described herein does not result in a second malignancy in the human.

In some embodiments, responses (i.e., stable disease response, partial response, complete response, objective response, best overall response, best complete response, best partial response, survival, progression-free survival, or overall survival) are assessed according to the Modified Lugano Response Criteria for Malignant Lymphoma (Cheson et al. (2014) “Recommendations for Initial Evaluation, Staging and Response Assessment of Hodgkin and Non-Hodgkin Lymphoma: The Lugano Classification.” J. Clin Oncol. 32:1-9). In some embodiments, the Modified Lugano Response criteria include that the designation of a complete response (CR) using positron emission tomography and computed tomography (PET-CT) requires normal bone marrow by morphology (if indeterminate by morphology, immunohistochemistry [IHC] should be negative) for humans with bone marrow involvement prior to the start of treatment according to any of the methods described herein. In some embodiments, the Modified Lugano Response criteria include that the designation of PET-CT based partial response (PR) requires that CT-based response criteria for a CR or PR be met in addition to the PET-CT based response criteria for a PR. In some embodiments, the therapeutic response is assessed according to the Modified Lugano Response Criteria for Malignant Lymphoma (Cheson et al. 2014), as described in Example 1 herein.

In some embodiments, a complete response according to the Modified Lugano Response Criteria for Malignant Lymphoma (Cheson et al. 2014) based on positron emission tomography-computed tomography (PET-CT) includes one or all of the following: (i) a score of 1, 2, or 3 with or without a residual mass on 5-point scale (5PS) at lymph nodes and extralymphatic sites. A score of 3 in many patients indicates a good prognosis with standard treatment, especially if at the time of an interim scan (e.g., during treatment). However, in trials involving PET where de-escalation is investigated, it may be preferable to consider a score of 3 as inadequate response (to avoid undertreatment). Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (e.g., liver, spleen, kidneys, lungs), GI involvement, cutaneous lesions, or those noted on palpation. Non-measured lesions: Any disease not selected as measured; dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors). It is recognized that in Waldeyer's ring or extranodal sites with high physiologic uptake or with activation within spleen or marrow, e.g., with chemotherapy or myeloid colony-stimulating factors, uptake may be greater than normal mediastinum and/or liver. In this circumstance, complete metabolic response may be inferred if uptake at sites of initial involvement is no greater than surrounding normal tissue even if the tissue has high physiologic uptake; PET 5PS: 1=no uptake above background; 2=uptake ≤mediastinum; 3=uptake >mediastinum but ≤liver; 4=uptake moderately >liver; 5=uptake markedly higher than liver and/or new lesions; X=new areas of uptake unlikely to be related to lymphoma; (ii) no new lesions; and (iii) in the bone marrow, no evidence of FDG-avid disease in marrow. In some embodiments, a complete response according to the Modified Lugano Response Criteria for Malignant Lymphoma (Cheson et al. 2014) based on PET-CT is referred to as complete metabolic response. In some embodiments, a complete response according to the Modified Lugano Response Criteria for Malignant Lymphoma (Cheson et al. 2014) based on computed tomography (CT) includes all of the following: (i) at lymph nodes and extralymphatic sites, target nodes/nodal masses must regress to ≤1.5 cm in longest transverse diameter of a lesion (LDi); (ii) at lymph nodes and extralymphatic sites, no extralymphatic sites of disease; (iii) absent non-measured lesions; (iv) organ enlargement regressed to normal; (v) no new lesions; and (vi) normal bone marrow by morphology; if indeterminate, IHC negative. In some embodiments, a complete response according to the Modified Lugano Response Criteria for Malignant Lymphoma (Cheson et al. 2014) based on CT is referred to as complete radiologic response. In some embodiments, designation of a complete response using positron emission tomography and computed tomography (PET-CT) requires normal bone marrow by morphology for patients with bone marrow involvement at baseline (if indeterminate by morphology, immunohistochemistry should be negative).

In some embodiments, a response of stable disease according to the Modified Lugano Response Criteria for Malignant Lymphoma (Cheson et al. 2014) based on positron emission tomography-computed tomography (PET-CT) includes one or all of the following: (i) a score 4 or 5 with no significant change in fluorodeoxyglucose (FDG) uptake from prior to the start of treatment at target nodes/nodal masses, extranodal lesions; PET 5-point scale (5PS): 1=no uptake above background; 2=uptake ≤mediastinum; 3=uptake >mediastinum but ≤liver; 4=uptake moderately >liver; 5=uptake markedly higher than liver and/or new lesions; X=new areas of uptake unlikely to be related to lymphoma; (ii) no new lesions; and (iii) no changes from baseline in bone marrow. In some embodiments, a response of stable disease according to the Modified Lugano Response Criteria for Malignant Lymphoma (Cheson et al. 2014) based on PET-CT is referred to as no metabolic response. In some embodiments, a response of stable disease according to the Modified Lugano Response Criteria for Malignant Lymphoma (Cheson et al. 2014) based on computed tomography (CT) includes one or all of the following: (i) <50% decrease from baseline in sum of the product of the perpendicular diameters for multiple (SPD) of up to 6 dominant, measurable nodes and extranodal sites, and no criteria for progressive disease being met at target nodes/nodal masses, extranodal lesions; (ii) no increases consistent with progression at non-measured lesions; (iii) no increases consistent with progression of organ enlargements; and (iv) no new lesions. In some embodiments, a response of stable disease according to the Modified Lugano Response Criteria for Malignant Lymphoma (Cheson et al. 2014) based on CT is referred to as stable disease.

In some embodiments, a partial response according to the Modified Lugano Response Criteria for Malignant Lymphoma (Cheson et al. 2014) based on positron emission tomography-computed tomography (PET-CT) includes one or all of the following: (i) a score of 4 or 5 with reduced uptake compared with mass(es) of any size prior to treatment or with residual masses at lymph nodes and extralymphatic sites (during treatment, these findings suggest responding disease; at end of treatment, these findings indicate residual disease); PET 5PS: 1=no uptake above background; 2=uptake ≤mediastinum; 3=uptake >mediastinum but ≤liver; 4=uptake moderately >liver; 5=uptake markedly higher than liver and/or new lesions; X=new areas of uptake unlikely to be related to lymphoma; (ii) no new lesions; and (iii) in bone marrow, residual uptake higher than uptake in normal marrow but reduced compared with prior to treatment (diffuse uptake compatible with reactive changes from chemotherapy is allowed). If there are persistent focal changes in the marrow in the context of a nodal response, consideration is given to further evaluation with MRI or biopsy or an interval scan. In some embodiments, a partial response according to the Modified Lugano Response Criteria for Malignant Lymphoma (Cheson et al. 2014) based on PET-CT is referred to as partial metabolic response. In some embodiments, a partial response according to the Modified Lugano Response Criteria for Malignant Lymphoma (Cheson et al. 2014) based on computed tomography (CT) includes all of the following: (i) ≥50% decrease in SPD of up to 6 target measurable nodes and extranodal sites in lymph nodes and extralymphatic sites (when a lesion is too small to measure on CT, 5 mm×5 mm is assigned as the default value; when a lesion is no longer visible, 0×0 mm is assigned; for a node >5 mm×5 mm but smaller than normal, the actual measurement is used for calculation); (ii) absent/normal or regressed non-measured lesions, but no increase in non-measured lesions; (iii) spleen must have regressed by >50% in length beyond normal; and (iv) no new lesions. In some embodiments, a partial response according to the Modified Lugano Response Criteria for Malignant Lymphoma (Cheson et al. 2014) based on CT is referred to as partial remission. In some embodiments, designation of PET-CT based partial response requires that CT-based response criteria for a complete response or partial response be met in addition to the PET-CT based response criteria for a partial response.

In some embodiments, disease progression according to the Modified Lugano Response Criteria for Malignant Lymphoma (Cheson et al. 2014) based on positron emission tomography-computed tomography (PET-CT) includes one or all of the following: (i) a score of 4 or 5 with an increase in intensity of uptake from prior to treatment at individual target nodes/nodal masses and/or new FDG-avid foci consistent with lymphoma during treatment or at the end of treatment at extranodal lesions; PET 5PS: 1=no uptake above background; 2=uptake ≤mediastinum; 3=uptake >mediastinum but ≤liver; 4=uptake moderately >liver; 5=uptake markedly higher than liver and/or new lesions; X=new areas of uptake unlikely to be related to lymphoma; (ii) new FDG-avid foci consistent with lymphoma rather than another etiology (e.g., infection, inflammation), if uncertain regarding etiology of new lesions, biopsy or interval scan may be considered; and (iii) in the bone marrow, new or recurrent FDG-avid foci. In some embodiments, disease progression according to the Modified Lugano Response Criteria for Malignant Lymphoma (Cheson et al. 2014) based on PET-CT is referred to as progressive metabolic disease. In some embodiments, disease progression according to the Modified Lugano Response Criteria for Malignant Lymphoma (Cheson et al. 2014) based on computed tomography (CT) includes at least one of the following: (i) cross product of the LDi and perpendicular diameter (PPD) progression at individual target nodes/nodal masses; (ii) at extranodal lesions, an individual node/lesion must be abnormal with: LDi>1.5 cm, and increase by >50% from PPD nadir, and an increase in LDi or shortest axis perpendicular to the LDi (SDi) from nadir, 0.5 cm for lesions ≤2 cm, 1.0 cm for lesions >2 cm; (iii) in the setting of splenomegaly, the splenic length must increase by >50% of the extent of its prior increase beyond baseline (e.g., a 15-cm spleen must increase to >16 cm). If no prior splenomegaly, must increase by at least 2 cm from baseline; (iv) new or recurrent splenomegaly; (v) new or clear progression of preexisting non-measured lesions; (vi) regrowth of previously resolved lesions; (vii) a new node >1.5 cm in any axis; (viii) a new extranodal site >1.0 cm in any axis; if <1.0 cm in any axis, its presence must be unequivocal and must be attributable to lymphoma; (ix) new lesions of assessable disease of any size unequivocally attributable to lymphoma; (x) at bone marrow, new or recurrent involvement. In some embodiments, disease progression according to the Modified Lugano Response Criteria for Malignant Lymphoma (Cheson et al. 2014) based on CT is referred to as progressive disease. In some embodiments, disease progression is determined on the basis of CT-scans alone or death from any cause.

In some embodiments, a best overall response refers to the best response of a complete response or a partial response during or after treatment according to any of the methods described herein. Thus, a human that achieves a best overall response, has achieved a best response of a complete response (i.e., a best complete response) or a partial response (i.e., a best partial response) during or after treatment according to any of the methods described herein. In some embodiments, best complete response is assessed according to the criteria described herein for the assessment of complete responses. In some embodiments, best partial response is assessed according to the criteria described herein for the assessment of partial responses.

In some embodiments, an objective response refers to a complete response or a partial response during or after treatment according to any of the methods described herein. Thus, a human that achieves an objective response, has achieved a complete response or a partial response during or after treatment according to any of the methods described herein. In some embodiments, objective responses are assessed according to the criteria described herein for the assessment of complete responses or partial responses.

In some embodiments, the duration of a response (i.e., of a stable disease response, partial response, complete response, objective response, best overall response, best complete response, or best partial response) is assessed from the time of the first occurrence of the response (i.e., the stable disease response, partial response, complete response, objective response, best overall response, best complete response, or best partial response) to the time of an occurrence of one or all of treatment failure, including disease progression or relapse, initiation of new anti-lymphoma therapy, and/or death from any cause (whichever occurs first).

In some embodiments, progression-free survival (PFS) or absence of disease progression is assessed as the time from initiation of treatment according to the methods provided herein, to first occurrence of disease progression or relapse, or death from any cause.

In some embodiments, survival is assessed as the time from initiation of treatment according to the methods provided herein, to death from any cause. In some embodiments, overall survival is assessed as the time from initiation of treatment according to the methods provided herein, to death from any cause.

Further details regarding clinical staging of and response criteria for lymphomas such as DLBCL are provided in, e.g., Van Heertum et al. (2017) Drug Des. Devel. Ther. 11: 1719-1728; Cheson et al. (2016) Blood. 128: 2489-2496; Cheson et al. (2014) J. Clin. Oncol. 32(27): 3059-3067; Barrington et al. (2017) J. Clin. Oncol. 32(27): 3048-3058; Gallamini et al. (2014) Haematologica. 99(6): 1107-1113; Barrinton et al. (2010) Eur. J. Nucl. Med. Mol. Imaging. 37(10): 1824-33; Moskwitz (2012) Hematology Am Soc. Hematol. Educ. Program 2012: 397-401; and Follows et al. (2014) Br. J. Haematology 166: 34-49. The progress of any one of the methods of treatment provided herein can be monitored by techniques known in the art.

In some embodiments, the human is an adult. In some embodiments, the human has received one therapy for DLBCL prior to the start of treatment according to any of the methods described herein. In some embodiments, the human has received at least one therapy for DLBCL prior to the start of treatment according to any of the methods described herein. In some embodiments, the human has received at least two therapies for DLBCL prior to the start of treatment according to any of the methods described herein. In some embodiments, the human has received a therapy for DLBCL comprising a chemoimmunotherapy that included an anti-CD20 antibody prior to the start of treatment according to any of the methods described herein. In some embodiments, the human has been administered a prior bone marrow transplant for DLBCL prior to the start of treatment according to any of the methods described herein. In some embodiments, the human has been administered a chimeric antigen receptor (CAR)-T-cell therapy for DLBCL prior to the start of treatment according to any of the methods described herein. In some embodiments, the human has DLBCL that was refractory to the first treatment for DLBCL administered to the human prior to the start of treatment according to any of the methods described herein. In some embodiments, the human has DLBCL that was refractory to the most recent prior therapy for DLBCL prior to the start of treatment according to any of the methods described herein. In some embodiments, the human has DLBCL that was not refractory to the most recent prior therapy for DLBCL prior to the start of treatment according to any of the methods described herein. In some embodiments, the human has an Eastern Cooperative Oncology Group (ECOG) Performance Status of 0, 1, or 2 prior to the start of treatment according to any of the methods described herein. In some embodiments, the human has DLBCL with an Ann Arbor Stage III or IV prior to the start of treatment according to any of the methods described herein. In some embodiments, the human has DLBCL with an International Prognostic Index of between 3 and 5 prior to the start of treatment according to any of the methods described herein. In some embodiments, the human has relapsed or refractory DLBCL (R/R DLBCL) prior to the start of treatment according to any of the methods described herein. In some embodiments, the human has bulky disease (e.g., ≥7 cm). In some embodiments, the human has DLBCL with a cell of origin (COO) of germinal center B-cell (GCB). In some embodiments, the human has DLBCL with a cell of origin (COO) of activated B cell (ABC). In some embodiments COO is assessed using any suitable method known in the art, such as gene expression profiling (e.g., using microarrays), immunohistochemistry, or digital gene expression profiling (e.g., NanoString). In some embodiments, the human has DLBCL that overexpresses B-cell lymphoma 2 (BCL-2). In some embodiments, the human has DLBCL that overexpresses MYC. In some embodiments, the human has DLBCL that overexpresses MYC and BCL-2 (i.e., double-expressor or DEL). In some embodiments, the human does not have double-expressor DLBCL. In some embodiments, expression of MYC and/or BCL-2 is assessed using any suitable method known in the art, such as ELISA, immunoblots, flow cytometry, mass spectrometry or immunohistochemistry. In some embodiments, the human has R/R DLBCL after treatment with at least one prior chemoimmunotherapy regimen that included an anti-CD20 antibody (e.g., a monoclonal anti-CD20 antibody) prior to the start of treatment according to any of the methods described herein. In some embodiments, the human has R/R DLBCL and is not eligible for autologous stem-cell transplantation prior to the start of treatment according to any of the methods described herein. In some embodiments, the human has R/R DLBCL and experienced disease progression following treatment with high-dose chemotherapy plus autologous stem-cell transplantation prior to the start of treatment according to any of the methods described herein. In some embodiments, the human has histologically documented CD20-positive B-cell lymphoma prior to the start of treatment according to any of the methods described herein. In some embodiments, the human has fluorodeoxyglucose (FDG)-avid lymphoma (i.e., PET-positive lymphoma) prior to the start of treatment according to any of the methods described herein. In some embodiments, the human has at least one bi-dimensionally measurable lesion (e.g., greater than 1.5 cm in its largest dimension by computed tomography [CT] scan or magnetic resonance imaging [MRI]) prior to the start of treatment according to any of the methods described herein. In some embodiments, the human does not have Grade 3b follicular lymphoma prior to the start of treatment according to any of the methods described herein. In some embodiments, the human does not have history of transformation of indolent disease to DLBCL prior to the start of treatment according to any of the methods described herein. In some embodiments, the human does not have known CD20-negative status at relapse or progression prior to the start of treatment according to any of the methods described herein. In some embodiments, the human does not have central nervous system lymphoma or leptomeningeal infiltration. In some embodiments, the human has not had an allogeneic stem cell transplantation (SCT) prior to the start of treatment according to any of the methods described herein. In some embodiments, the human has not completed an autologous SCT within 100 days prior to the start of treatment according to any of the methods described herein. In some embodiments, the human does not have history of resistance to lenalidomide prior to the start of treatment according to any of the methods described herein. In some embodiments, the human does not have history of response to lenalidomide treatment with a duration of less than 1 year, prior to the start of treatment according to any of the methods described herein. In some embodiments, the human is not taking or has not been administered lenalidomide, fludarabine, or alemtuzumab within 12 months prior to the start of treatment according to any of the methods described herein. In some embodiments, the human is not taking or has not been administered a radioimmunoconjugate within 12 weeks prior to the start of treatment according to any of the methods described herein. In some embodiments, the human is not taking or has not been administered a monoclonal antibody or antibody-drug conjugate (ADC) therapy within 5 half-lives or 4 weeks prior to the start of treatment according to any of the methods described herein. In some embodiments, the human is not taking or has not been administered a radiotherapy, a chemotherapy, a hormonal therapy, or a targeted small-molecule therapy within 2 weeks prior to the start of treatment according to any of the methods described herein. In some embodiments, the human does not have a clinically significant toxicity (other than alopecia) from a prior therapy that has not resolved to Grade ≤2 (per NCI CTCAE, Version 4.0) prior to the start of treatment according to any of the methods described herein. In some embodiments, the human is not taking or has not been administered a systemic immunosuppressive medication, e.g., prednisone, azathioprine, methotrexate, thalidomide, or anti-tumor necrosis factor agents, within 2 weeks prior to the start of treatment according to any of the methods described herein. In some embodiments, the human does not have history of severe allergic or anaphylactic reaction to humanized or murine monoclonal antibodies prior to the start of treatment according to any of the methods described herein. In some embodiments, the human does not have known sensitivity or allergy to murine products or any component of rituximab, polatuzumab vedotin, or lenalidomide formulations prior to the start of treatment according to any of the methods described herein. In some embodiments, the human does not have history of erythema multiforme, Grade ≥3 rash, or desquamation (blistering) following prior treatment with immunomodulatory derivatives such as thalidomide and lenalidomide prior to the start of treatment according to any of the methods described herein. In some embodiments, the human does not have an active bacterial, viral, fungal, or other infection prior to the start of treatment according to any of the methods described herein. In some embodiments, the human is not positive for hepatitis B surface antigen (HBsAg), total hepatitis B core antibody (HBcAb), or hepatitis C virus (HCV) antibody prior to the start of treatment according to any of the methods described herein. In some embodiments, the human does not have known history of HIV positive status prior to the start of treatment according to any of the methods described herein. In some embodiments, the human has not been administered a vaccination with a live virus vaccine prior to the start of treatment according to any of the methods described herein. In some embodiments, the human does not have history of progressive multifocal leukoencephalopathy prior to the start of treatment according to any of the methods described herein. In some embodiments, the human does not have contraindication to treatment for thromboembolism (TE) prophylaxis prior to the start of treatment according to any of the methods described herein. In some embodiments, the human does not have Grade ≥2 neuropathy prior to the start of treatment according to any of the methods described herein. In some embodiments, the human does not have inadequate hematologic function (e.g., hemoglobin <9 g/dL; absolute neutrophil count (ANC)<1.5×10⁹/L; and/or platelet count <75×10⁹/L), unless due to underlying lymphoma, prior to the start of treatment according to any of the methods described herein. In some embodiments, the human does not have calculated creatinine clearance <50 mL/min (using the Cockcroft-Gault formula), unless due to underlying lymphoma, prior to the start of treatment according to any of the methods described herein. In some embodiments, the human does not have aspartate aminotransferase (AST) or alanine transaminase (ALT)>2.5×upper limit of normal (ULN), unless due to underlying lymphoma, prior to the start of treatment according to any of the methods described herein. In some embodiments, the human does not have serum total bilirubin >1.5×ULN (or >3×ULN for humans with Gilbert syndrome), unless due to underlying lymphoma, prior to the start of treatment according to any of the methods described herein. In some embodiments, the human does not have international normalized ratio (INR) or prothrombin time (PT)>1.5×ULN in the absence of therapeutic anticoagulation, unless due to underlying lymphoma, prior to the start of treatment according to any of the methods described herein. In some embodiments, the human does not have evidence of significant, uncontrolled concomitant disease, including significant cardiovascular disease (e.g., such as New York Heart Association Class III or IV cardiac disease, myocardial infarction within the previous 6 months, unstable arrhythmia, or unstable angina), or significant pulmonary disease (e.g., obstructive pulmonary disease or history of bronchospasm) prior to the start of treatment according to any of the methods described herein. In some embodiments, the human does not have another malignancy prior to the start of treatment according to any of the methods described herein, except for curatively treated carcinoma in situ of the cervix, good-prognosis ductal carcinoma in situ of the breast, basal- or squamous-cell skin cancer, Stage I melanoma, low-grade and early-stage localized prostate cancer, or any previously treated malignancy that has been in remission without treatment for ≥2 years prior to start of treatment according to any of the methods described herein. In some embodiments, the human does not have partial thromboplastin time (PTT) or activated partial thromboplastin time (aPTT)>1.5×ULN in the absence of a lupus anticoagulant, unless due to underlying lymphoma, prior to the start of treatment according to any of the methods described herein.

### IV. Immunoconjugates Comprising an Anti-CD79b Antibody and a Drug/Cytotoxic Agent (“Anti-CD79b Immunoconjugates”)

In some embodiments, the anti-CD79b immunoconjugate comprises an anti-CD79b antibody (Ab) which targets a cancer cell (such as a diffuse large B-cell lymphoma (DLBCL) cell), a drug moiety (D), and a linker moiety (L) that attaches Ab to D. In some embodiments, the anti-CD79b antibody is attached to the linker moiety (L) through one or more amino acid residues, such as lysine and/or cysteine. In some formula Ab-(L-D)p, wherein: (a) Ab is the anti-CD79b antibody which binds CD79b on the surface of a cancer cell (e.g., a DLBCL cell); (b) L is a linker; (c) D is a cytotoxic agent; and (d) p ranges from 1-8.

An exemplary anti-CD79b immunoconjugate comprises Formula I:

Ab-(L-D)ₚ (I)

wherein p is 1 to about 20 (e.g., 1 to 15, 1 to 10, 1 to 8, 2 to 5, or 3 to 4). In some embodiments, the number of drug moieties that can be conjugated to the anti-CD79b antibody is limited by the number of free cysteine residues. In some embodiments, free cysteine residues are introduced into the antibody amino acid sequence by the methods described elsewhere herein. Exemplary anti-CD79b immunoconjugates of Formula I comprise, but are not limited to, anti-CD79b antibodies that comprise 1, 2, 3, or 4 engineered cysteine amino acids (Lyon, R. et al (2012) Methods in Enzym. 502:123-138). In some embodiments, one or more free cysteine residues are already present in the anti-CD79b antibody, without the use of engineering, in which case the existing free cysteine residues may be used to conjugate the anti-CD79b antibody to the drug/cytotoxic agent. In some embodiments, the anti-CD79b antibody is exposed to reducing conditions prior to conjugation of the antibody to the drug/cytotoxic agent in order to generate one or more free cysteine residues.

A. Exemplary Linkers

A “linker” (L) is a bifunctional or multifunctional moiety that can be used to link one or more drug moieties (D) to the anti-CD79b antibody (Ab) to form an anti-CD79b immunoconjugate of Formula I. In some embodiments, anti-CD79b immunoconjugate can be prepared using a linker having reactive functionalities for covalently attaching to the drug and to the anti-CD79b antibody. For example, in some embodiments, a cysteine thiol of the anti-CD79b antibody (Ab) can form a bond with a reactive functional group of a linker or a drug-linker intermediate to make the anti-CD79b immunoconjugate.

In one aspect, a linker has a functionality that is capable of reacting with a free cysteine present on the anti-CD79b antibody to form a covalent bond. Exemplary reactive functionalities include, without limitation, e.g., maleimide, haloacetamides, a-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a linker has a functionality that is capable of reacting with an electrophilic group present on the anti-CD79b antibody. Exemplary electrophilic groups include, without limitation, e.g., aldehyde and ketone carbonyl groups. In some embodiments, a heteroatom of the reactive functionality of the linker can react with an electrophilic group on an antibody and form a covalent bond to an antibody unit. Exemplary reactive functionalities include, but are not limited to, e.g., hydrazide, oxime, amino, hydrazine, thiosemicarbazone, hydrazine carboxylate, and arylhydrazide.

In some embodiments, the linker comprises one or more linker components. Exemplary linker components include, e.g., 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl) cyclohexane-1 carboxylate (“MCC”). Various linker components are known in the art, some of which are described below.

In some embodiments, the linker is a “cleavable linker,” facilitating release of a drug. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020).

In certain embodiments, a linker (L) has the following Formula II:

-Aₐ-W_{w}-Y_{y}- (II)

wherein A is a “stretcher unit,” and a is an integer from 0 to 1; W is an “amino acid unit,” and w is an integer from 0 to 12; Y is a “spacer unit,” and y is 0, 1, or 2; and Ab, D, and p are defined as above for Formula I. Exemplary embodiments of such linkers are described in U.S. Pat. No. 7,498,298, which is expressly incorporated herein by reference.

In some embodiments, a linker component comprises a “stretcher unit” that links an antibody to another linker component or to a drug moiety. Nonlimiting exemplary stretcher units are shown below (wherein the wavy line indicates sites of covalent attachment to an antibody, drug, or additional linker components):

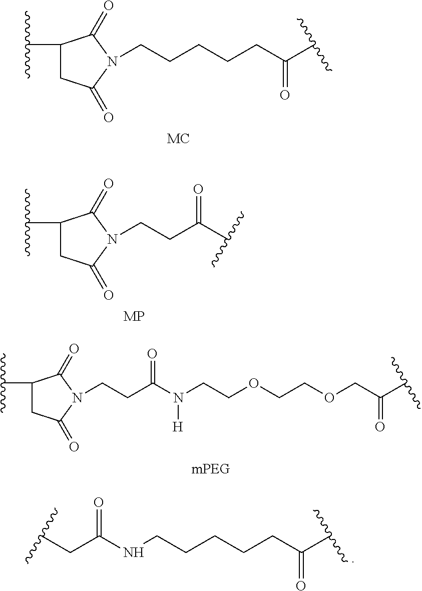 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component comprises an “amino acid unit.” In some such embodiments, the amino acid unit allows for cleavage of the linker by a protease, thereby facilitating release of the drug/cytotoxic agent from the anti-CD79b immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, cathepsin B, C and D, or a plasmin protease.

In some embodiments, a linker component comprises a “spacer” unit that links the antibody to a drug moiety, either directly or through a stretcher unit and/or an amino acid unit. A spacer unit may be “self-immolative” or a “non-self-immolative.” A “non-self-immolative” spacer unit is one in which part or all of the spacer unit remains bound to the drug moiety upon cleavage of the ADC. Examples of non-self-immolative spacer units include, but are not limited to, a glycine spacer unit and a glycine-glycine spacer unit. In some embodiments, enzymatic cleavage of an ADC containing a glycine-glycine spacer unit by a tumor-cell associated protease results in release of a glycine-glycine-drug moiety from the remainder of the ADC. In some such embodiments, the glycine-glycine-drug moiety is subjected to a hydrolysis step in the tumor cell, thus cleaving the glycine-glycine spacer unit from the drug moiety.

A “self-immolative” spacer unit allows for release of the drug moiety. In certain embodiments, a spacer unit of a linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the spacer unit is p-aminobenzyloxycarbonyl (PAB). In some embodiments, an anti-CD79b immunoconjugate comprises a self-immolative linker that comprises the structure:

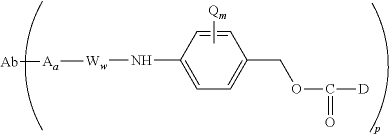

wherein Q is —C₁-C₅ alkyl, —O—(C₁-C₅ alkyl), -halogen, -nitro, or -cyno; m is an integer ranging from 0 to 4; and p ranges from 1 to about 20. In some embodiments, p ranges from 1 to 10, 1 to 7, 1 to 5, or 1 to 4.

Other examples of self-immolative spacers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Linkage of a drug to the a-carbon of a glycine residue is another example of a self-immolative spacer that may be useful in ADC (Kingsbury et al (1984) J. Med. Chem. 27:1447).

In some embodiments, linker L may be a dendritic type linker for covalent attachment of more than one drug moiety to an antibody through a branching, multifunctional linker moiety (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where an antibody bears only one reactive cysteine thiol group, a multitude of drug moieties may be attached through a dendritic linker.

Nonlimiting exemplary linkers are shown below in the context of an anti-CD79 immunoconjugates of Formulas III, IV, V:

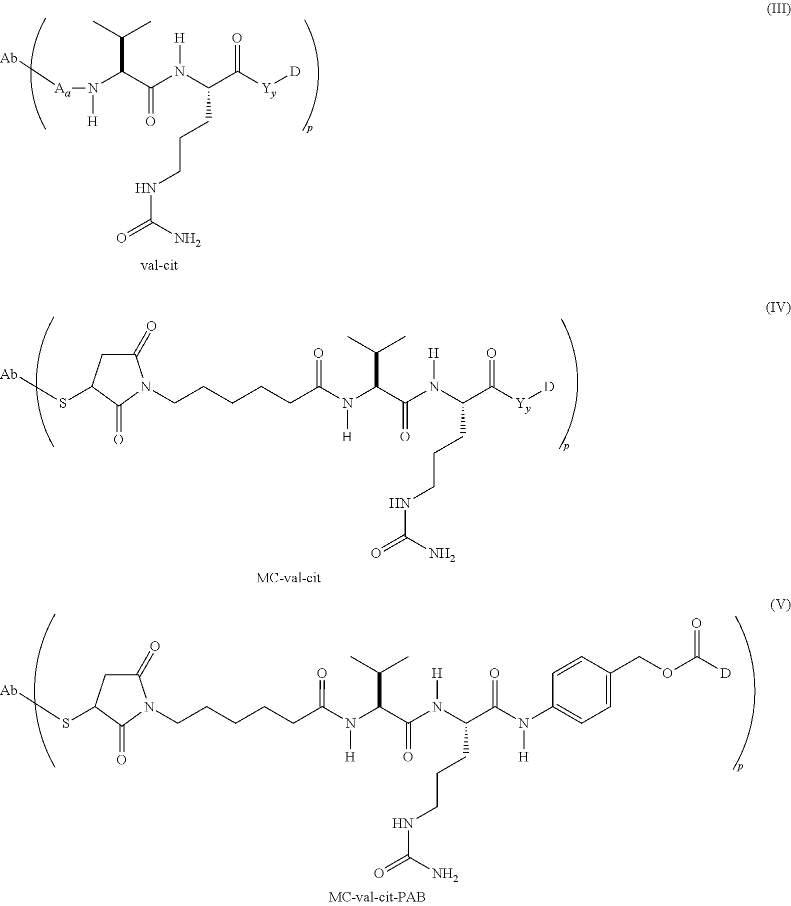

Wherein (Ab) is an anti-CD79b antibody, (D) is a drug/cytotoxic agent, “Val-Cit” is a valine-citrulline dipeptide, MC is 6-maleimidocaproyl, PAB is p-aminobenzyloxycarbonyl, and p is 1 to about 20 (e.g., 1 to 15, 1 to 10, 1 to 8, 2 to 5, or 3 to 4).

In some embodiments, the anti-CD79b immunoconjugate comprises a structure of any one of formulas VI-V below:

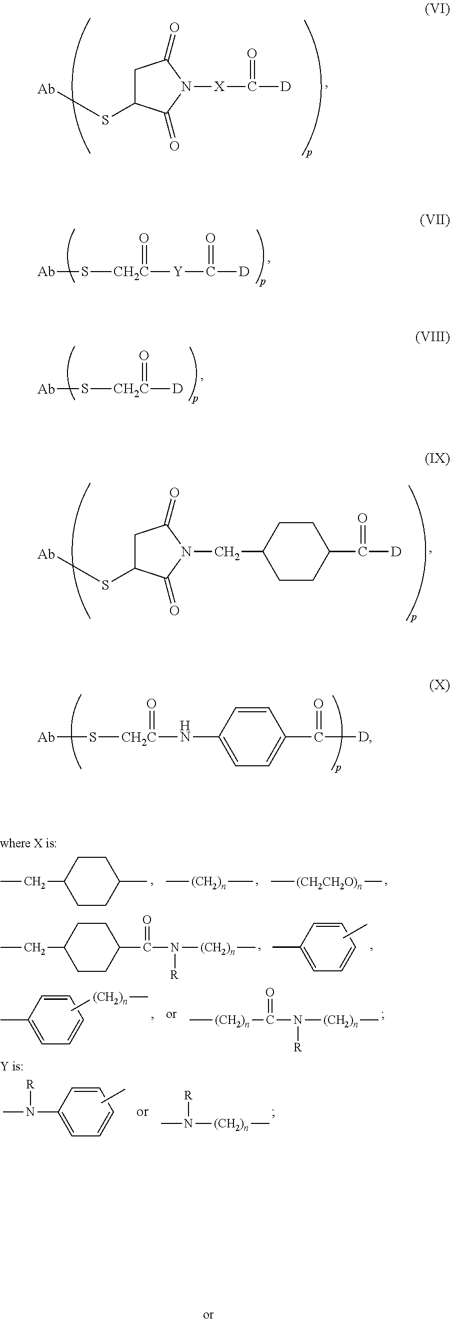

each R is independently H or C₁-C₆ alkyl; and n is 1 to 12.

Typically, peptide-type linkers can be prepared by forming a peptide bond between two or more amino acids and/or peptide fragments. Such peptide bonds can be prepared, for example, according to a liquid phase synthesis method (e.g., E. Schröder and K. Lübke (1965) “The Peptides”, volume 1, pp 76-136, Academic Press).

In some embodiments, a linker is substituted with groups that modulate solubility and/or reactivity. As a non-limiting example, a charged substituent such as sulfonate (—SO₃—) or ammonium may increase water solubility of the linker reagent and facilitate the coupling reaction of the linker reagent with the antibody and/or the drug moiety, or facilitate the coupling reaction of Ab-L (anti-CD79b antibody-linker intermediate) with D, or D-L (drug/cytotoxic agent-linker intermediate) with Ab, depending on the synthetic route employed to prepare the anti-CD79b immunoconjugate. In some embodiments, a portion of the linker is coupled to the antibody and a portion of the linker is coupled to the drug, and then the anti-CD79 Ab-(linker portion)^{a} is coupled to drug/cytotoxic agent-(linker portion)b to form the anti-CD79b immunoconjugate of Formula I. In some such embodiments, the anti-CD79b antibody comprises more than one (linker portion)^{a} substituents, such that more than one drug/cytotoxic agent is coupled to the anti-CD79b antibody in the anti-CD79b immunoconjugate of Formula I.

The anti-CD79b immunoconjugates provided herein expressly contemplate, but are not limited to, anti-CD79b immunoconjugates prepared with the following linker reagents: bis-maleimido-trioxyethylene glycol (BMPEO), N-(D-maleimidopropyloxy)-N-hydroxy succinimide ester (BMPS), N-(E-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(beta-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, and sulfo-SMPB, and succinimidyl-(4-vinylsulfone)benzoate (SVSB), and including bis-maleimide reagents: dithiobismaleimidoethane (DTME), 1,4-Bismaleimidobutane (BMB), 1,4 Bismaleimidyl-2,3-dihydroxybutane (BMDB), bismaleimidohexane (BMH), bismaleimidoethane (BMOE), BM(PEG)₂ (shown below), and BM(PEG)₃ (shown below); bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In some embodiments, bis-maleimide reagents allow the attachment of the thiol group of a cysteine in the antibody to a thiol-containing drug moiety, linker, or linker-drug intermediate. Other functional groups that are reactive with thiol groups include, but are not limited to, iodoacetamide, bromoacetamide, vinyl pyridine, disulfide, pyridyl disulfide, isocyanate, and isothiocyanate.

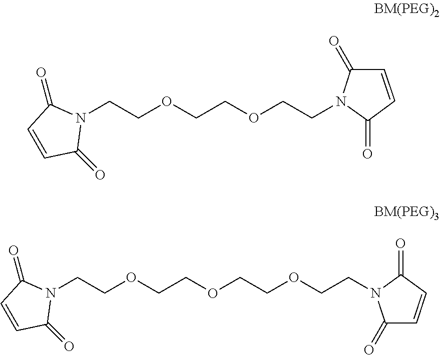 [SMILES: O=C1C=CC(=O)N1CCOCCOCCN1C(=O)C=CC1=O ; O=C1C=CC(=O)N1CCOCCOCCOCCN1C(=O)C=CC1=O]

Certain useful linker reagents can be obtained from various commercial sources, such as Pierce Biotechnology, Inc. (Rockford, IL), Molecular Biosciences Inc. (Boulder, CO), or synthesized in accordance with procedures described in the art; for example, in Toki et al (2002) J. Org. Chem. 67:1866-1872; Dubowchik, et al. (1997) Tetrahedron Letters, 38:5257-60; Walker, M. A. (1995) J. Org. Chem. 60:5352-5355; Frisch et al (1996) Bioconjugate Chem. 7:180-186; U.S. Pat. No. 6,214,345; WO 02/088172; US 2003130189; US2003096743; WO 03/026577; WO 03/043583; and WO 04/032828.

Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026.

B. Anti-CD79b Antibodies

In some embodiments, the immunoconjugate (e.g., anti-CD79b immunoconjugate) comprises an anti-CD79b antibody that comprises at least one, two, three, four, five, or six HVRs selected from (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some such embodiments, the immunoconjugate comprises an anti-CD79 antibody comprising at least one of: (i) HVR-H3 comprising the amino acid sequence of SEQ ID NO: 23, and/or (ii) HVR-L1 comprising an amino acid sequence of SEQ ID NO: 24. In some embodiments, the immunoconjugate comprises an anti-CD79 antibody comprising at least one of: (i) HVR-H3 comprising the amino acid sequence of SEQ ID NO: 23, and/or (ii) HVR-L1 comprising the amino acid sequence of SEQ ID NO: 24. In some embodiments, the immunoconjugate comprises an anti-CD79b antibody comprising at least one, at least two, or all three VH HVR sequences selected from (a) HVR-H1 comprising the amino acid sequence of SEQ ID NO: 21; (b) HVR-H2 comprising the amino acid sequence of SEQ ID NO: 22; and (c) HVR-H3 comprising the amino acid sequence of SEQ ID NO: 23. In some embodiments, the immunoconjugate comprises an anti-CD79b antibody that comprises an HVR-H3 comprising the amino acid sequence of SEQ ID NO: 23. In some embodiments, the immunoconjugate comprises an anti-CD79b antibody that comprises an HVR-H3 comprising the amino acid sequence of SEQ ID NO: 23 and an HVR-L3 comprising the amino acid sequence of SEQ ID NO: 26. In some embodiments, the immunoconjugate comprises an anti-CD79b antibody that comprises an HVR-H3 comprising the amino acid sequence of SEQ ID NO: 23, an HVR-L3 comprising the amino acid sequence of SEQ ID NO: 26, and an HVR-H2 comprising the amino acid sequence of SEQ ID NO: 22. In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; and (c) HVR-H3 comprising the amino acid sequence of SEQ ID NO: 23.

In some embodiments, the immunoconjugate comprises an anti-CD79b antibody comprising at least one, at least two, or all three VL HVR sequences selected from (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises an anti-CD79b antibody that comprises at least one, at least two, or all three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises an anti-CD79b antibody that comprises an HVR-L1 comprising the amino acid sequence of SEQ ID NO: 24 In some embodiments, the immunoconjugate comprises an anti-CD79b antibody that comprises (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, the immunoconjugate comprises an anti-CD79b antibody comprising (a) a VH domain comprising at least one, at least two, or all three VH HVR sequences selected from (i) HVR-H1 comprising the amino acid sequence of SEQ ID NO: 21, (ii) HVR-H2 comprising the amino acid sequence of SEQ ID NO: 22, and (iii) HVR-H3 comprising an amino acid sequence selected from SEQ ID NO:23; and (b) a VL domain comprising at least one, at least two, or all three VL HVR sequences selected from (i) HVR-L1 comprising an amino acid sequence of SEQ ID NO: 24, (ii) HVR-L2 comprising the amino acid sequence of SEQ ID NO: 25, and (iii) HVR-L3 comprising the amino acid sequence of SEQ ID NO: 26. In some embodiments, the immunoconjugate comprises an anti-CD79b antibody that comprises at least one of: (i) HVR-H3 comprising the amino acid sequence of SEQ ID NO: 23, and/or (ii) HVR-L1 comprising the amino acid sequence of SEQ ID NO: 24.

In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some embodiments, the immunoconjugate comprises at least one of: HVR-H3 comprising the amino acid sequence of SEQ ID NO: 23 and/or HVR-L1 comprising an amino acid sequence of SEQ ID NO: 24. In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, the anti-CD79b immunoconjugates comprises a humanized anti-CD79b antibody. In some embodiments, an anti-CD79b antibody comprises HVRs as in any of the embodiments provided herein, and further comprises a human acceptor framework, e.g., a human immunoglobulin framework or a human consensus framework. In some embodiments, the human acceptor framework is the human VL kappa 1 (VL_{KI}) framework and/or the VH framework VH_{III}. In some embodiments, a humanized anti-CD79b antibody comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some embodiments, a humanized anti-CD79b antibody comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, the immunoconjugate (e.g., the anti-CD79b immunoconjugate) comprises an anti-CD79 antibody comprising a heavy chain variable domain (VH) sequence having at least 90%, 91%, 92%, 93%, 94%, 95%, 96%, 97%, 98%, 99%, or 100% sequence identity to the amino acid sequence of SEQ ID NO: 19. In some embodiments, a VH sequence having at least 90%, 91%, 92%, 93%, 94%, 95%, 96%, 97%, 98%, or 99% identity to the amino acid sequence of SEQ ID NO: 19 contains substitutions (e.g., conservative substitutions), insertions, or deletions relative to the reference sequence, but an anti-CD79b immunoconjugate comprising that sequence retains the ability to bind to CD79b. In some embodiments, a total of 1 to 10 amino acids have been substituted, inserted and/or deleted in SEQ ID NO: 19. In some embodiments, a total of 1 to 5 amino acids have been substituted, inserted and/or deleted in SEQ ID NO: 19. In some embodiments, substitutions, insertions, or deletions occur in regions outside the HVRs (i.e., in the FRs). In some embodiments, the immunoconjugate (e.g., the anti-CD79b immunoconjugate) comprises the VH sequence of SEQ ID NO: 19, including post-translational modifications of that sequence. In some embodiments, the VH comprises one, two or three HVRs selected from: (a) HVR-H1 comprising the amino acid sequence of SEQ ID NO: 21, (b) HVR-H2 comprising the amino acid sequence of SEQ ID NO: 22, and (c) HVR-H3 comprising the amino acid sequence of SEQ ID NO: 23.

In some embodiments, the immunoconjugate (e.g., the anti-CD79b immunoconjugate) comprises an anti-CD79b antibody that comprises a light chain variable domain (VL) having at least 90%, 91%, 92%, 93%, 94%, 95%, 96%, 97%, 98%, 99%, or 100% sequence identity to the amino acid sequence of SEQ ID NO: 20. In certain embodiments, a VL sequence having at least 90%, 91%, 92%, 93%, 94%, 95%, 96%, 97%, 98%, or 99% identity to the amino acid sequence of SEQ ID NO: 20 contains substitutions (e.g., conservative substitutions), insertions, or deletions relative to the reference sequence, but an anti-CD79b immunoconjugate comprising that sequence retains the ability to bind to CD79b. In certain embodiments, a total of 1 to 10 amino acids have been substituted, inserted and/or deleted in SEQ ID NO: 20. In certain embodiments, a total of 1 to 5 amino acids have been substituted, inserted and/or deleted in SEQ ID NO: 20. In certain embodiments, the substitutions, insertions, or deletions occur in regions outside the HVRs (i.e., in the FRs). In some embodiments, the anti-CD79b immunoconjugate comprises an anti-CD79b antibody that comprises the VL sequence of SEQ ID NO: 20, including post-translational modifications of that sequence. In some embodiments, the VL comprises one, two or three HVRs selected from (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the VL comprises one, two or three HVRs selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, the immunoconjugate (e.g., the anti-CD79b immunoconjugate) comprises an anti-CD79b antibody that comprises VH as in any of the embodiments provided herein, and a VL as in any of the embodiments provided herein. In some embodiments, the immunoconjugate comprises an anti-CD79b antibody that comprises the VH and VL sequences in SEQ ID NO: 19 and SEQ ID NO: 20, respectively, including post-translational modifications of those sequences.

In some embodiments, the immunoconjugate (e.g., anti-CD79b immunoconjugate) comprises an anti-CD79b antibody that binds to the same epitope as an anti-CD79b antibody described herein. For example, in some embodiments, the immunoconjugate (e.g., anti-CD79b immunoconjugate) comprises an anti-CD79b antibody that binds to the same epitope as an anti-CD79b antibody comprising a VH sequence of SEQ ID NO: 19 and a VL sequence of SEQ ID NO: 20.

In some embodiments, the immunoconjugate comprises an anti-CD79b antibody that is a monoclonal antibody, a chimeric antibody, humanized antibody, or human antibody. In some embodiments, immunoconjugate comprises an antigen-binding fragment of an anti-CD79b antibody described herein, e.g., a Fv, Fab, Fab′, scFv, diabody, or F(ab′)₂ fragment. In some embodiments, the immunoconjugate comprises a substantially full length anti-CD79b antibody, e.g., an IgG1 antibody or other antibody class or isotype as described elsewhere herein.

In some embodiments, the immunoconjugate comprises an anti-CD79b antibody comprising a heavy chain comprising the amino acid sequence of SEQ ID NO: 36, and wherein the light chain comprises the amino acid sequence of SEQ ID NO: 35. In some embodiments, the immunoconjugate comprises an anti-CD79 antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 38.

In some embodiments, the immunoconjugate is polatuzumab vedotin, as described in WHO Drug Information, Vol. 26, No. 4, 2012 (Proposed INN: List 108), which is expressly incorporated by reference herein in its entirety. As shown in WHO Drug Information, Vol. 26, No. 4, 2012, polatuzumab vedotin has the following structure: immunoglobulin G1-kappa auristatin E conjugate, anti-[Homo sapiens CD79B (immunoglobulin-associated CD79 beta)], humanized monoclonal antibody conjugated to auristatin E; gamma1 heavy chain (1-447) [humanized VH (Homo sapiens IGHV3-66*01 (79.60%)—(IGHD)-IGHJ4*01) [8.8.13] (1-120) -Homo sapiens IGHG1*03 (CHI R120>K (214) (121-218), hinge (219-233), CH2 (234-343), CH3 (344-448), CHS (449-450)) (121-450)], (220-218′)-disulfide (if not conjugated) with kappa light chain (1′-218′) [humanized V-KAPPA (Homo sapiens IGKV1-39*01 (80.00%) -IGKJ1*01) [11.3.9] (1′-112′) -Homo sapiens IGKC*01 (113′-218′)]; dimer (226-226″:229-229″)-bisdisulfide; conjugated, on an average of 3 to 4 cysteinyl, to monomethylauristatin E (MMAE), via a cleavable maleimidecaproyl-valyl-citrullinyl-p-aminobenzylcarbamate (mc-val-cit-PABC) linker; the heavy chain of polatuzumab vedotin has the following sequence:

| (SEQ ID NO: 56) | |
|---|---|
| EVQLVESGGG LVQPGGSLRL SCAASGYTFS SYWIEWVRQA PGKGLEWIGE | 50 |
| ILPGGGDTNY NEIFKGRATF SADTSKNTAY LQMNSLRAED TAVYYCTRRV | 100 |
| PIRLDYWGQG TLVTVSSAST KGPSVFPLAP SSKSTSGGTA ALGCLVKDYF | 150 |
| PEPVTVSWNS GALTSGVHTF PAVLQSSGLY SLSSVVTVPS SSLGTQTYIC | 200 |
| NVNHKPSNTK VDKKVEPKSC DKTHTCPPCP APELLGGPSV FLFPPKPKDT | 250 |
| LMISRTPEVT CVVVDVSHED PEVKFNWYVD GVEVHNAKTK PREEQYNSTY | 300 |
| RVVSVLTVLH QDWLNGKEYK CKVSNKALPA PIEKTISKAK GQPREPQVYT | 350 |
| LPPSREEMTK NQVSLTCLVK GFYPSDIAVE WESNGQPENN YKTTPPVLDS | 400 |
| DGSFFLYSKL TVDKSRWQQG NVFSCSVMHE ALHNHYTQKS LSLSPGK; | 447 |
| the light chain of polatuzumab vedotin has the following sequence: | |
| (SEQ ID NO: 35) | |
| DIQLTQSPSS LSASVGDRVT ITCKASQSVD YEGDSFLNWY QQKPGKAPKL | 50 |
| LIYAASNLES GVPSRFSGSG SGTDFTLTIS SLQPEDFATY YCQQSNEDPL | 100 |
| TFGQGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC; | 218 |

the disulfide bridge locations are:
Intra-H 22-96 144-200 261-321 367-425
   22″-96″ 147″-203″ 261″-321″ 367″-425″
Intra-L 23′-92′ 138′-198′
   23′″-92′″138′″-198′″
Inter-H-L* 220-218′ 220″-218′″
Inter-H-H* 226-226″ 229-229″
*Two or three of the inter-chain disulfide bridges are not present, the antibody being conjugated to an average of 3 to 4 drug linkers each via a thioether bond;
the N-glycosylation sites are H CH2 N84.4: 297, 297″ but lacking carbohydrate;
and other post-translational modifications are: lacking H chain C-terminal lysine. Thus, in some embodiments, the heavy chain of polatuzumab vedotin has the sequence of SEQ ID NO: 36.

C. Drugs/Cytotoxic Agents

Anti-CD79 immunoconjugates comprise an anti-CD79b antibody (e.g., an anti-CD79b antibody described herein) conjugated to one or more drugs/cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes (i.e., a radioconjugate). Such immunoconjugates are targeted chemotherapeutic molecules which combine properties of both antibodies and cytotoxic drugs by targeting potent cytotoxic drugs to antigen-expressing cancer cells (such as tumor cells) (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107. That is, the anti-CD79 immunoconjugates selectively deliver an effective dose of a drug to cancerous cells/tissues whereby greater selectivity, i.e. a lower efficacious dose, may be achieved while increasing the therapeutic index (“therapeutic window”) (Polakis P. (2005) Current Opinion in Pharmacology 5:382-387).

Anti-CD79 immunoconjugates used in the methods provided herein include those with anticancer activity. In some embodiments, the anti-CD79 immunoconjugate comprises an anti-CD79b antibody conjugated, i.e. covalently attached, to the drug moiety. In some embodiments, the anti-CD79b antibody is covalently attached to the drug moiety through a linker. The drug moiety (D) of t the anti-CD79 immunoconjugate may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, dolastatin, auristatin, calicheamicin, anthracycline, duocarmycin, vinca alkaloid, taxane, trichothecene, CC1065, camptothecin, elinafide, and stereoisomers, isosteres, analogs, and derivatives thereof that have cytotoxic activity.

(i) Maytansine and Maytansinoids

In some embodiments, an anti-CD79b immunoconjugate comprises an anti-CD79b antibody conjugated to one or more maytansinoid molecules. Maytansinoids are derivatives of maytansine, and are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinoids are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533. Maytansinoid drug moieties are attractive drug moieties in antibody-drug conjugates because they are: (i) relatively accessible to prepare by fermentation or chemical modification or derivatization of fermentation products, (ii) amenable to derivatization with functional groups suitable for conjugation through non-disulfide linkers to antibodies, (iii) stable in plasma, and (iv) effective against a variety of tumor cell lines.

Certain maytansinoids suitable for use as maytansinoid drug moieties are known in the art and can be isolated from natural sources according to known methods or produced using genetic engineering techniques (see, e.g., Yu et al (2002) PNAS 99:7968-7973). Maytansinoids may also be prepared synthetically according to known methods.

Exemplary maytansinoid drug moieties include, but are not limited to, those having a modified aromatic ring, such as: C-19-dechloro (U.S. Pat. No. 4,256,746) (prepared, for example, by lithium aluminum hydride reduction of ansamytocin P2); C-20-hydroxy (or C-20-demethyl)+/−C-19-dechloro (U.S. Pat. Nos. 4,361,650 and 4,307,016) (prepared, for example, by demethylation using Streptomyces or Actinomyces or dechlorination using LAH); and C-20-demethoxy, C-20-acyloxy (—OCOR), +/−dechloro (U.S. Pat. No. 4,294,757) (prepared, for example, by acylation using acyl chlorides), and those having modifications at other positions of the aromatic ring.

Exemplary maytansinoid drug moieties also include those having modifications such as: C-9-SH (U.S. Pat. No. 4,424,219) (prepared, for example, by the reaction of maytansinol with H₂S or P₂S5); C-14-alkoxymethyl(demethoxy/CH₂ OR)(U.S. Pat. No. 4,331,598); C-14-hydroxymethyl or acyloxymethyl (CH₂OH or CH₂OAc) (U.S. Pat. No. 4,450,254) (prepared, for example, from Nocardia); C-15-hydroxy/acyloxy (U.S. Pat. No. 4,364,866) (prepared, for example, by the conversion of maytansinol by Streptomyces); C-15-methoxy (U.S. Pat. Nos. 4,313,946 and 4,315,929) (for example, isolated from Trewia nudlflora); C-18-N-demethyl (U.S. Pat. Nos. 4,362,663 and 4,322,348) (prepared, for example, by the demethylation of maytansinol by Streptomyces); and 4,5-deoxy (U.S. Pat. No. 4,371,533) (prepared, for example, by the titanium trichloride/LAH reduction of maytansinol).

Many positions on maytansinoid compounds are useful as the linkage position. For example, an ester linkage may be formed by reaction with a hydroxyl group using conventional coupling techniques. In some embodiments, the reaction may occur at the C-3 position having a hydroxyl group, the C-14 position modified with hydroxymethyl, the C-15 position modified with a hydroxyl group, and the C-20 position having a hydroxyl group. In some embodiments, the linkage is formed at the C-3 position of maytansinol or a maytansinol analogue.

Maytansinoid drug moieties include those having the structure:

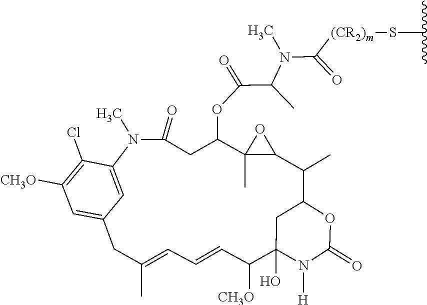

where the wavy line indicates the covalent attachment of the sulfur atom of the maytansinoid drug moiety to a linker of an anti-CD79b immunoconjugate. Each R may independently be H or a C₁-C₆ alkyl. The alkylene chain attaching the amide group to the sulfur atom may be methanyl, ethanyl, or propyl, i.e., m is 1, 2, or 3 (U.S. Pat. No. 633,410; U.S. Pat. No. 5,208,020; Chari et al (1992) Cancer Res. 52:127-131; Liu et al (1996) Proc. Natl. Acad. Sci USA 93:8618-8623).

All stereoisomers of the maytansinoid drug moiety are contemplated for the anti-CD79b immunoconjugate used in a method provided herein, i.e. any combination of R and S configurations at the chiral carbons (U.S. Pat. Nos. 7,276,497; 6,913,748; 6,441,163; 633,410 (RE39151); U.S. Pat. No. 5,208,020; Widdison et al (2006) J. Med. Chem. 49:4392-4408, which are incorporated by reference in their entirety). In some embodiments, the maytansinoid drug moiety has the following stereochemistry:

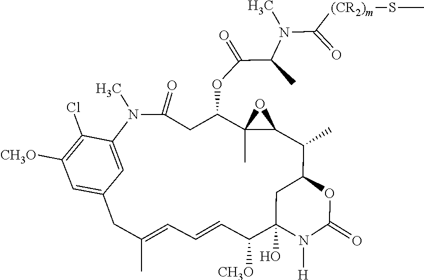

Exemplary embodiments of maytansinoid drug moieties include, but are not limited to, DM1; DM3; and DM4, having the structures:

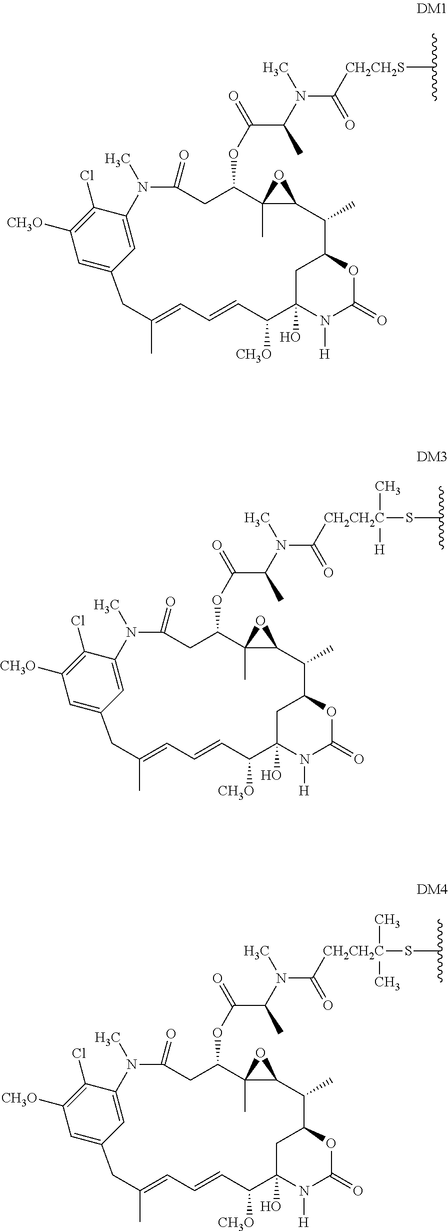

wherein the wavy line indicates the covalent attachment of the sulfur atom of the drug to a linker (L) of an anti-CD79b immunoconjugate.

Other exemplary maytansinoid anti-CD79b immunoconjugates have the following structures and abbreviations (wherein Ab is an anti-CD79b antibody and p is 1 to about 20. In some embodiments, p is 1 to 10, p is 1 to 7, p is 1 to 5, or p is 1 to 4):

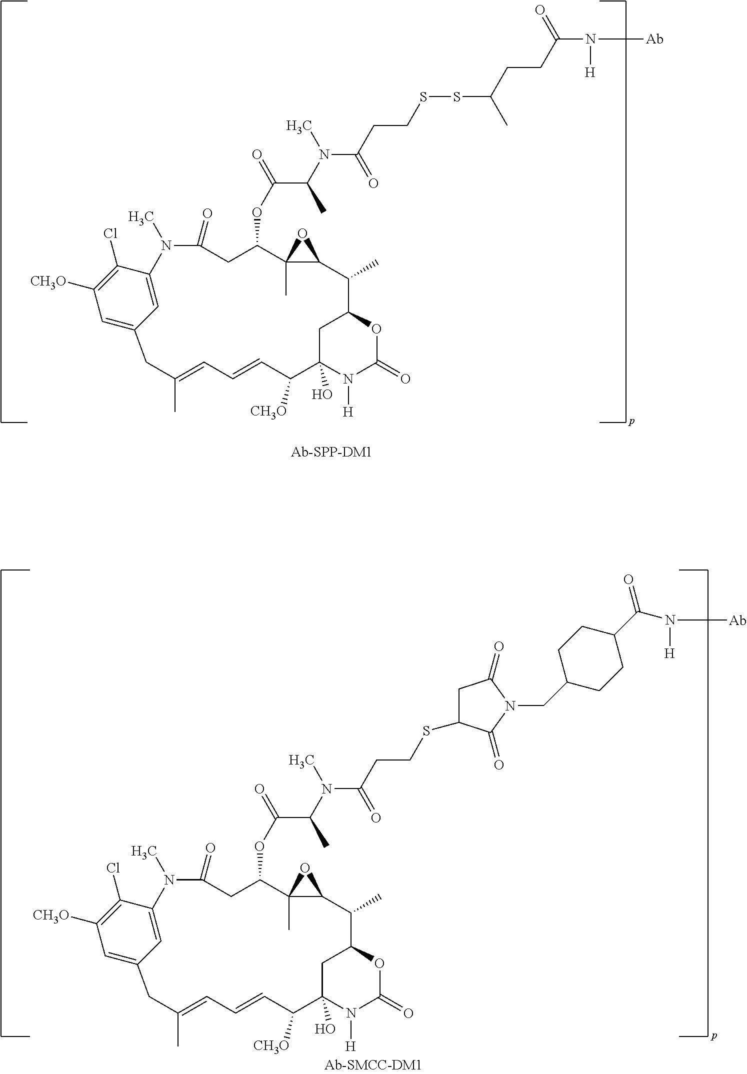

Exemplary antibody-drug conjugates where DM1 is linked through a BMPEO linker to a thiol group of the antibody have the structure and abbreviation:

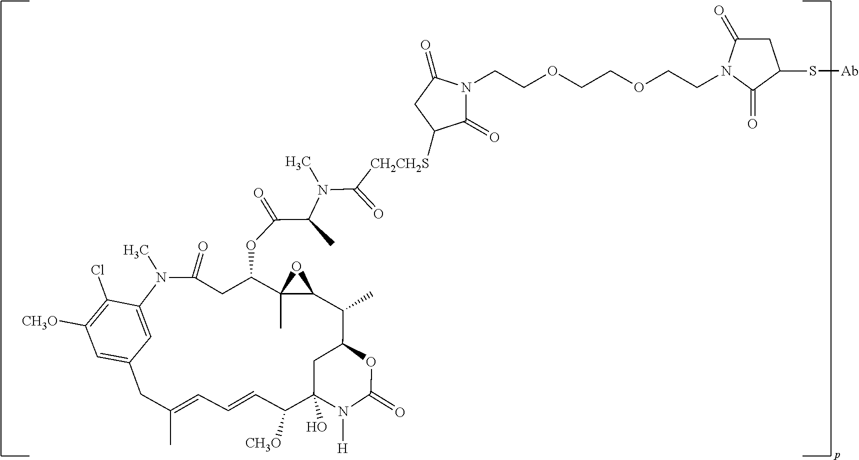

where Ab is an anti-CD79b antibody; n is 0, 1, or 2; and p is 1 to about 20. In some embodiments, p is 1 to 10, p is 1 to 7, p is 1 to 5, or p is 1 to 4.

Immunoconjugates containing maytansinoids, methods of making the same, and their therapeutic use are disclosed, for example, in U.S. Pat. Nos. 5,208,020 and 5,416,064; US 2005/0276812 A1; and European Patent EP 0 425 235 B1, the disclosures of which are hereby expressly incorporated by reference. See also Liu et al. Proc. Natl. Acad. Sci. USA 93:8618-8623 (1996); and Chari et al. Cancer Research 52:127-131 (1992).

In some embodiments, anti-CD79b antibody-maytansinoid conjugates may be prepared by chemically linking an anti-CD79b antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). In some embodiments, an anti-CD79b immunoconjugate with an average of 3-4 maytansinoid molecules conjugated per antibody molecule has shown efficacy in enhancing cytotoxicity of target cells without negatively affecting the function or solubility of the antibody. In some instances, even one molecule of toxin/antibody is expected to enhance cytotoxicity over the use of naked anti-CD79b antibody.

Exemplary linking groups for making antibody-maytansinoid conjugates include, for example, those described herein and those disclosed in U.S. Pat. No. 5,208,020; EP Patent 0 425 235 B1; Chari et al. Cancer Research 52:127-131 (1992); US 2005/0276812 A1; and US 2005/016993 A1, the disclosures of which are hereby expressly incorporated by reference.

(2) Auristatins and Dolastatins

Drug moieties include dolastatins, auristatins, and analogs and derivatives thereof (U.S. Pat. Nos. 5,635,483; 5,780,588; 5,767,237; 6,124,431). Auristatins are derivatives of the marine mollusk compound dolastatin-10. While not intending to be bound by any particular theory, dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin/auristatin drug moiety may be attached to the antibody through the N (amino) terminus or the C (carboxyl) terminus of the peptidic drug moiety (WO 02/088172; Doronina et al (2003) Nature Biotechnology 21(7):778-784; Francisco et al (2003) Blood 102(4):1458-1465).

Exemplary auristatin embodiments include the N-terminus linked monomethylauristatin drug moieties D_{E} and D_{F}, disclosed in U.S. Pat. Nos. 7,498,298 and 7,659,241, the disclosures of which are expressly incorporated by reference in their entirety:

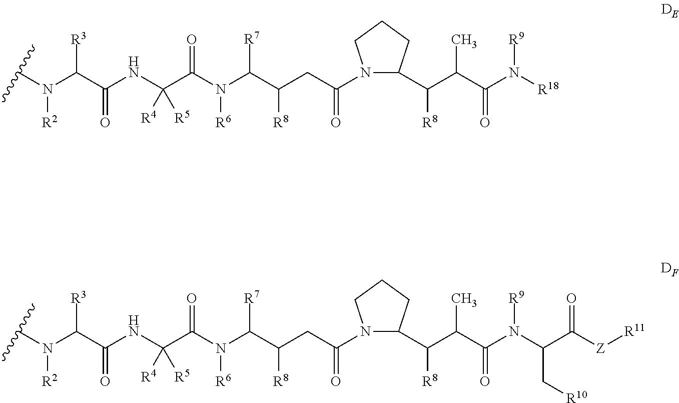

wherein the wavy line of D_{E} and D_{F} indicates the covalent attachment site to an antibody or antibody-linker component, and independently at each location:
R² is selected from H and C₁-C₅ alkyl;
R³ is selected from H, C₁-C₅ alkyl, C₃-C₈ carbocycle, aryl, C₁-C₅ alkyl-aryl, C₁-C₅ alkyl-(C₃-C₈ carbocycle), C₃-C₈ heterocycle and C₁-C₅ alkyl-(C₃-C₈ heterocycle);
R⁴ is selected from H, C₁-C₅ alkyl, C₃-C₈ carbocycle, aryl, C₁-C₅ alkyl-aryl, C₁-C₅ alkyl-(C₃-C₈ carbocycle), C₃-C₈ heterocycle and C₁-C₅ alkyl-(C₃-C₈ heterocycle);
R⁵ is selected from H and methyl;
or R⁴ and R⁵ jointly form a carbocyclic ring and have the formula —(CR^{a}R^{b})ₙ— wherein Ra and R^{b} are independently selected from H, C₁-C₅ alkyl and C₃-C₈ carbocycle and n is selected from 2, 3, 4, 5 and 6;
R⁶ is selected from H and C₁-C₅ alkyl;
R⁷ is selected from H, C₁-C₅ alkyl, C₃-C₈ carbocycle, aryl, C₁-C₅ alkyl-aryl, C₁-C₅ alkyl-(C₃-C₈ carbocycle), C₃-C₈ heterocycle and C₁-C₅ alkyl-(C₃-C₈ heterocycle);
each R⁸ is independently selected from H, OH, C₁-C₅ alkyl, C₃-C₈ carbocycle and O—(C₁-C₈ alkyl);
R⁹ is selected from H and C₁-C₅ alkyl;
R¹″ is selected from aryl or C₃-C₈ heterocycle;
Z is O, S, NH, or NR¹², wherein R¹² is C₁-C₅ alkyl;
R¹¹ is selected from H, C₁-C₂₀ alkyl, aryl, C₃-C₈ heterocycle, —(R¹³O)ₘ—R¹⁴, or —(R¹³O)ₘ—CH(R¹⁵)₂;
m is an integer ranging from 1-1000;
R¹³ is C₂-C₅ alkyl;
R¹⁴ is H or C₁-C₅ alkyl;
each occurrence of R¹⁵ is independently H, COOH, —(CH₂)ₙ—N(R¹⁶)₂, —(CH₂)ₙ—SO₃H, or —(CH2)ₙ— SO₃—C₁-C₅ alkyl;
each occurrence of R¹⁶ is independently H, C₁-C₅ alkyl, or —(CH₂)ₙ—COOH;
R¹⁸ is selected from —C(R⁸)₂—C(R⁸)₂-aryl, —C(R⁸)₂—C(R⁸)₂—(C₃-C₈ heterocycle), and —C(R⁸)₂—C(R⁸)₂—(C₃-C₈ carbocycle); and n is an integer ranging from 0 to 6.

In one embodiment, R³, R⁴ and R⁷ are independently isopropyl or sec-butyl and R⁵ is —H or methyl. In an exemplary embodiment, R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is -phenyl.

In an exemplary embodiment, when Z is —O—, R¹¹ is —H, methyl or t-butyl.

In one embodiment, when Z is —NH, R¹¹ is —CH(R¹⁵)₂, wherein R¹⁵ is —(CH₂)ₙ—N(R¹⁶)₂, and R¹⁶ is —C₁-C₅ alkyl or —(CH₂)ₙ—COOH.

In another embodiment, when Z is —NH, R¹¹ is —CH(R¹⁵)₂, wherein R¹⁵ is —(CH₂)ₙ—SO₃H.

An exemplary auristatin embodiment of formula D_{E} is MMAE, wherein the wavy line indicates the covalent attachment to a linker (L) of an anti-CD79b immunoconjugate:

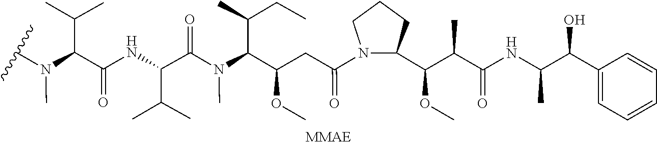 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula D_{F} is MMAF, wherein the wavy line indicates the covalent attachment to a linker (L) of an anti-CD79b immunoconjugate:

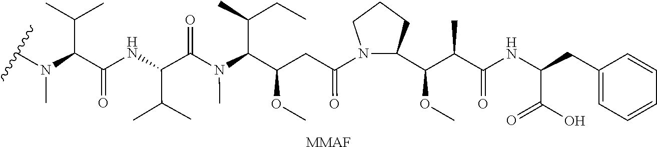

Other exemplary embodiments include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Nonlimiting exemplary embodiments of an anti-CD79b immunoconjugate of Formula I comprising MMAE or MMAF and various linker components have the following structures and abbreviations (wherein “Ab” is an anti-CD79b antibody; p is 1 to about 8, “Val-Cit” is a valine-citrulline dipeptide; and “S” is a sulfur atom:

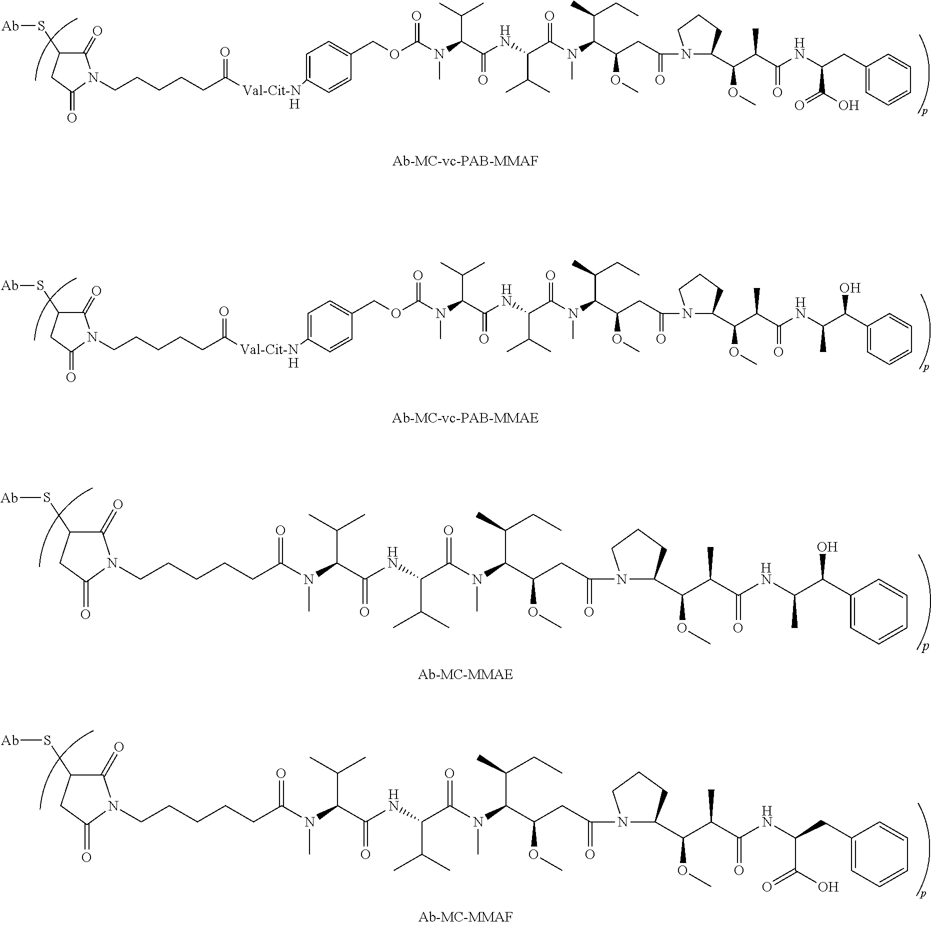

In certain embodiments, the anti-CD79b immunoconjugate comprises the structure of Ab-MC-vc-PAB-MMAE, wherein p is, e.g., about 1 to about 8; about 2 to about 7; about 3 to about 5; about 3 to about 4; or about 3.5. In some embodiments, the anti-CD79b immunoconjugate is huMA79bv28-MC-vc-PAB-MMAE, e.g., an anti-CD79b immunoconjugate comprising the structure of MC-vc-PAB-MMAE, wherein p is, e.g., about 1 to about 8; about 2 to about 7; about 3 to about 5; about 3 to about 4; or about 3.5, wherein the anti-CD79 antibody comprises a heavy chain comprising the amino acid sequence of SEQ ID NO: 36, and wherein the light chain comprises the amino acid sequence of SEQ ID NO: 35. In some embodiments, the anti-CD79b immunoconjugate is polatuzumab vedotin (CAS Number 1313206-42-6). Polatuzumab vedotin has the IUPHAR/BPS Number 8404, the KEGG Number D10761, the INN number 9714, and can also be referred to as “DCDS4501A,” or “RG7596.”

Nonlimiting exemplary embodiments of anti-CD79b immunoconjugates of Formula I comprising MMAF and various linker components further include Ab-MC-PAB-MMAF and Ab-PAB-MMAF. Immunoconjugates comprising MMAF attached to an antibody by a linker that is not proteolytically cleavable have been shown to possess activity comparable to immunoconjugates comprising MMAF attached to an antibody by a proteolytically cleavable linker (Doronina et al. (2006) Bioconjugate Chem. 17:114-124). In some such embodiments, drug release is believed to be effected by antibody degradation in the cell.

Typically, peptide-based drug moieties can be prepared by forming a peptide bond between two or more amino acids and/or peptide fragments. Such peptide bonds can be prepared, for example, according to a liquid phase synthesis method (see, e.g., E. Schroder and K. Lubke, “The Peptides”, volume 1, pp 76-136, 1965, Academic Press). Auristatin/dolastatin drug moieties may, in some embodiments, be prepared according to the methods of: U.S. Pat. Nos. 7,498,298; 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat. Biotechnol. 21(7):778-784.

In some embodiments, auristatin/dolastatin drug moieties of formulas D_{E} such as MMAE, and DF, such as MMAF, and drug-linker intermediates and derivatives thereof, such as MC-MMAF, MC-MMAE, MC-vc-PAB-MMAF, and MC-vc-PAB-MMAE, may be prepared using methods described in U.S. Pat. No. 7,498,298; Doronina et al. (2006) Bioconjugate Chem. 17:114-124; and Doronina et al. (2003) Nat. Biotech. 21:778-784 and then conjugated to an antibody of interest.

(3) Calicheamicin

In some embodiments, the anti-CD79b immunoconjugate comprises an anti-CD79b antibody conjugated to one or more calicheamicin molecules. The calicheamicin family of antibiotics, and analogues thereof, are capable of producing double-stranded DNA breaks at sub-picomolar concentrations (Hinman et al., (1993) Cancer Research 53:3336-3342; Lode et al., (1998) Cancer Research 58:2925-2928). Calicheamicin has intracellular sites of action but, in certain instances, does not readily cross the plasma membrane. Therefore, cellular uptake of these agents through antibody-mediated internalization may, in some embodiments, greatly enhance their cytotoxic effects. Nonlimiting exemplary methods of preparing anti-CD79b antibody immunoconjugates with a calicheamicin drug moiety are described, for example, in U.S. Pat. Nos. 5,712,374; 5,714,586; 5,739,116; and 5,767,285.

(4) Other Drug Moieties

In some embodiments, an anti-CD79b immunoconjugate comprises geldanamycin (Mandler et al (2000) J. Nat. Cancer Inst. 92(19):1573-1581; Mandler et al (2000) Bioorganic & Med. Chem. Letters 10:1025-1028; Mandler et al (2002) Bioconjugate Chem. 13:786-791); and/or enzymatically active toxins and fragments thereof, including, but not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, e.g., WO 93/21232.

Drug moieties also include compounds with nucleolytic activity (e.g., a ribonuclease or a DNA endonuclease).

In certain embodiments, an anti-CD79b immunoconjugate comprises a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. In some embodiments, when an anti-CD79b immunoconjugate is used for detection, it may comprise a radioactive atom for scintigraphic studies, for example Tc⁹⁹ or I¹²³, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

The radio- or other labels may be incorporated in the anti-CD79b immunoconjugate in known ways. For example, a peptide may be biosynthesized or chemically synthesized using suitable amino acid precursors comprising, for example, one or more fluorine-19 atoms in place of one or more hydrogens. In some embodiments, labels such as Tc⁹⁹, I¹²³, Re¹⁸⁶, Re¹⁸⁸ and In¹¹¹ can be attached via a cysteine residue in the anti-CD79b antibody. In some embodiments, yttrium-90 can be attached via a lysine residue of the anti-CD79b antibody. In some embodiments, the IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes certain other methods.

In certain embodiments, an anti-CD79b immunoconjugate may comprise an anti-CD79b antibody conjugated to a prodrug-activating enzyme. In some such embodiments, a prodrug-activating enzyme converts a prodrug (e.g., a peptidyl chemotherapeutic agent, see WO 81/01145) to an active drug, such as an anti-cancer drug. Such immunoconjugates are useful, in some embodiments, in antibody-dependent enzyme-mediated prodrug therapy (“ADEPT”). Enzymes that may be conjugated to an anti-CD79b antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibodies by recombinant DNA techniques well known in the art. See, e.g., Neuberger et al., Nature 312:604-608 (1984).

D. Drug Loading

Drug loading is represented by p, the average number of drug moieties per anti-CD79b antibody in a molecule of Formula I. Drug loading may range from 1 to 20 drug moieties (D) per antibody. Anti-CD79b immunoconjugates of Formula I include collections of anti-CD79b antibodies conjugated with a range of drug moieties, from 1 to 20. The average number of drug moieties per anti-CD79b antibody in preparations of anti-CD79b immunoconjugates from conjugation reactions may be characterized by conventional means such as mass spectroscopy, ELISA assay, and HPLC. The quantitative distribution of anti-CD79b immunoconjugates in terms of p may also be determined. In some instances, separation, purification, and characterization of homogeneous anti-CD79b immunoconjugates where p is a certain value from anti-CD79b immunoconjugates with other drug loadings may be achieved by means such as reverse phase HPLC or electrophoresis.

For some anti-CD79b immunoconjugates, p may be limited by the number of attachment sites on the anti-CD79b antibody. For example, where the attachment is a cysteine thiol, as in certain exemplary embodiments above, an anti-CD79b antibody may have only one or several cysteine thiol groups, or may have only one or several sufficiently reactive thiol groups through which a linker may be attached. In certain embodiments, higher drug loading, e.g., p>5, may cause aggregation, insolubility, toxicity, or loss of cellular permeability of certain anti-CD79b immunoconjugates. In certain embodiments, the average drug loading for an anti-CD79b immunoconjugates ranges from 1 to about 8; from about 2 to about 6; from about 3 to about 5; or from about 3 to about 4. Indeed, it has been shown that for certain antibody-drug conjugates, the optimal ratio of drug moieties per antibody may be less than 8, and may be about 2 to about 5 (U.S. Pat. No. 7,498,298). In certain embodiments, the optimal ratio of drug moieties per antibody is about 3 to about 4. In certain embodiments, the optimal ratio of drug moieties per antibody is about 3.5.

In certain embodiments, fewer than the theoretical maximum of drug moieties are conjugated to the anit-CD79b antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an anti-CD79b antibody may be reduced with a reducing agent such as dithiothreitol (DTT) or tricarbonylethylphosphine (TCEP), under partial or total reducing conditions, to generate reactive cysteine thiol groups. In certain embodiments, an anti-CD79b antibody is subjected to denaturing conditions to reveal reactive nucleophilic groups such as lysine or cysteine.

The loading (drug/antibody ratio) of an anti-CD79b immunoconjugate may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent, then the resulting product is a mixture of anti-CD79b immunoconjugate compounds with a distribution of one or more drug moieties attached to an anti-CD79b antibody. The average number of drugs per antibody may be calculated from the mixture by a dual ELISA antibody assay, which is specific for antibody and specific for the drug. Individual anti-CD79b immunoconjugate molecules may be identified in the mixture by mass spectroscopy and separated by HPLC, e.g., hydrophobic interaction chromatography (see, e.g., McDonagh et al (2006) Prot. Engr. Design & Selection 19(7):299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous anti-CD79b immunoconjugate with a single loading value may be isolated from the conjugation mixture by electrophoresis or chromatography.

E. Methods of Preparing Anti-CD79b Immunoconjugates

An anti-CD79b immunoconjugate of Formula I may be prepared by several routes employing organic chemistry reactions, conditions, and reagents known to those skilled in the art, including, but not limited to, e.g., (1) reaction of a nucleophilic group of an anti-CD79b antibody with a bivalent linker reagent to form Ab-L via a covalent bond, followed by reaction with a drug moiety D; and (2) reaction of a nucleophilic group of a drug moiety with a bivalent linker reagent, to form D-L, via a covalent bond, followed by reaction with a nucleophilic group of an anti-CD79b antibody. Exemplary methods for preparing an anti-CD79b immunoconjugate of Formula I via the latter route are described in U.S. Pat. No. 7,498,298, which is expressly incorporated herein by reference.

Nucleophilic groups on antibodies include, but are not limited to: (i)N-terminal amine groups, (ii) side chain amine groups, e.g., lysine, (iii) side chain thiol groups, e.g., cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, i.e. cysteine bridges. Anti-CD79b antibodies may be made reactive for conjugation with linker reagents by treatment with a reducing agent such as DTT (dithiothreitol) or tricarbonylethylphosphine (TCEP), such that the anti-CD79b antibody is fully or partially reduced. Each cysteine bridge will thus form, theoretically, two reactive thiol nucleophiles. Additional nucleophilic groups can be introduced into anti-CD79b antibodies through modification of lysine residues, e.g., by reacting lysine residues with 2-iminothiolane (Traut's reagent), resulting in conversion of an amine into a thiol. Reactive thiol groups may also be introduced into an anti-CD79b antibody by introducing one, two, three, four, or more cysteine residues (e.g., by preparing variant antibodies comprising one or more non-native cysteine amino acid residues).

Anti-CD79b immunoconjugates described herein may also be produced by reaction between an electrophilic group on an anti-CD79b antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an anti-CD79b antibody is modified to introduce electrophilic moieties that are capable of reacting with nucleophilic substituents on the linker reagent or drug. In another embodiment, the sugars of glycosylated anti-CD79b antibodies may be oxidized, e.g., with periodate oxidizing reagents, to form aldehyde or ketone groups which may react with the amine group of linker reagents or drug moieties. The resulting imine Schiff base groups may form a stable linkage, or may be reduced, e.g., by borohydride reagents to form stable amine linkages. In one embodiment, reaction of the carbohydrate portion of a glycosylated anti-CD79b antibody with either galactose oxidase or sodium meta-periodate may yield carbonyl (aldehyde and ketone) groups in the anti-CD79b antibody that can react with appropriate groups on the drug (Hermanson, Bioconjugate Techniques). In another embodiment, anti-CD79b antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a drug moiety or linker nucleophile.

Exemplary nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linker reagents that may be used to prepare anti-CD79b immunoconjugates are described herein in the section titled “Exemplary Linkers.” Methods of using such cross-linker reagents to link two moieties, including a proteinaceous moiety and a chemical moiety, are known in the art. In some embodiments, a fusion protein comprising an anti-CD79b antibody and a cytotoxic agent may be made, e.g., by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate. In yet another embodiment, an anti-CD79b antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a drug or radionucleotide). Additional details regarding anti-CD79b immunoconjugates are provided in U.S. Pat. No. 8,545,850 and WO/2016/049214, the contents of which are expressly incorporated by reference herein in their entirety.

In some embodiments, provided is an immunoconjugate comprising the formula

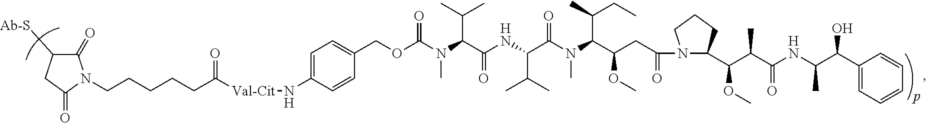

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating diffuse large B-cell lymphoma (DLBCL), e.g., relapsed/refractory DLBCL, in an individual (a human individual) in need thereof, the method comprising administering to the individual an effective amount of the immunoconjugate, an immunomodulatory agent (e.g., lenalidomide), and an anti-CD20 antibody (e.g., obinutuzumab or rituximab). In some embodiments, the individual achieves at least stable disease (SD) (e.g., at least SD, at least partial response (PR) or a complete response (CR)) during or following treatment with the immunoconjugate, the immunomodulatory drug (e.g., lenalidomide), and the anti-CD20 antibody (e.g., obinutuzumab or rituximab). In some embodiments, the immunoconjugate is for use in a method described herein. In some embodiments, the immunoconjugate comprises an anti-CD79b antibody comprising (i) a VH comprising the amino acid sequence of SEQ ID NO: 19 and (ii) a VL comprising the amino acid sequence of SEQ ID NO: 20. In some embodiments, the immunoconjugate is polatuzumab vedotin.

In some embodiments, provided is a use of an immunoconjugate comprising the formula

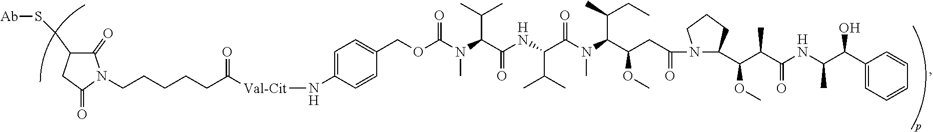

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 in the manufacture of a medicament for treating diffuse large B-cell lymphoma (DLBCL), e.g., relapsed/refractory DLBCL, in an individual (a human individual) in need thereof, wherein the medicament is for (e.g., formulated for) administration in combination with an immunomodulatory agent (e.g., lenalidomide), and an anti-CD20 antibody (e.g., obinutuzumab or rituximab). In some embodiments, the individual achieves at least stable disease (SD) (e.g., at least SD, at least partial response (PR) or a complete response (CR)) during or following treatment with the medicament, the immunomodulatory drug (e.g., lenalidomide), and the anti-CD20 antibody (e.g., obinutuzumab or rituximab). In some embodiments, the medicament (i.e., the medicament comprising the immunoconjugate) is for use in a method described herein. In some embodiments, the immunoconjugate comprises an anti-CD79b antibody comprising (i) a VH comprising the amino acid sequence of SEQ ID NO: 19 and (ii) a VL comprising the amino acid sequence of SEQ ID NO: 20. In some embodiments, the immunoconjugate is polatuzumab vedotin.

In some embodiments, provided is an immunoconjugate comprising the formula

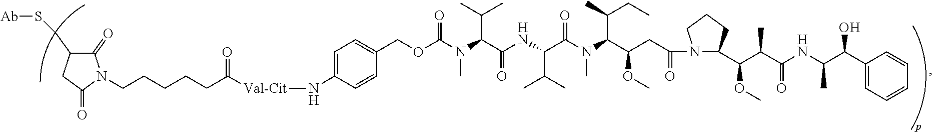

wherein Ab is an anti-CD79b antibody that comprises (i) a VH comprising the amino acid sequence of SEQ ID NO: 19 and (ii) a VL comprising the amino acid sequence of SEQ ID NO: 20, and wherein p is between 2 and 5, for use in a method of treating diffuse large B-cell lymphoma (DLBCL), e.g., relapsed/refractory DLBCL, in an individual (a human individual) in need thereof, the method comprising administering to the individual an effective amount of (a) the immunoconjugate, (b) lenalidomide, and (c) obinutuzumab, wherein the immunoconjugate is administered at a dose between about 1.4 and about 1.8 mg/kg, the lenalidomide is administered at a dose between about 10 mg and 20 mg, and the obinutuzumab is administered at a dose 1000 mg. In some embodiments, the individual achieves at least stable disease (SD) (e.g., at least SD, at least partial response (PR) or a complete response (CR)) during or following treatment with the immunoconjugate, the lenalidomide, and the obinutuzumab. In some embodiments, the immunoconjugate is for use according to a method described herein. In some embodiments, p is between 3 and 4. In some embodiments, p is 3.5. In some embodiments, p is 3.4. In some embodiments, the immunoconjugate comprises an anti-CD79b antibody comprising a heavy chain comprises the amino acid sequence of SEQ ID NO: 36, and wherein the light chain comprises the amino acid sequence of SEQ ID NO: 35. In some embodiments, the immunoconjugate comprises an anti-CD79 antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 38. In some embodiments, the immunoconjugate is polatuzumab vedotin.

In some embodiments, provided is an immunoconjugate comprising the formula

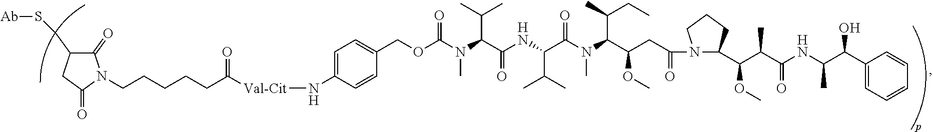

wherein Ab is an anti-CD79b antibody that comprises (i) a VH comprising the amino acid sequence of SEQ ID NO: 19 and (ii) a VL comprising the amino acid sequence of SEQ ID NO: 20, and wherein p is between 2 and 5, for use in a method of treating diffuse large B-cell lymphoma (DLBCL), e.g., relapsed/refractory DLBCL, in an individual (a human individual) in need thereof, the method comprising administering to the individual an effective amount of (a) the immunoconjugate, (b) lenalidomide, and (c) rituximab, wherein the immunoconjugate is administered at a dose between about 1.4 and about 1.8 mg/kg, the lenalidomide is administered at a dose between about 10 mg and 20 mg, and the rituximab is administered at a dose of about 375 mg/m². In some embodiments, the individual achieves at least stable disease (SD) (e.g., at least SD, at least partial response (PR) or a complete response (CR)) during or following treatment with the immunoconjugate, lenalidomide, and the rituximab. In some embodiments, the immunoconjugate is for use according to a method described herein. In some embodiments, p is between 3 and 4. In some embodiments, p is 3.5. In some embodiments, p is 3.4. In some embodiments, the immunoconjugate comprises an anti-CD79b antibody comprising a heavy chain comprises the amino acid sequence of SEQ ID NO: 36, and wherein the light chain comprises the amino acid sequence of SEQ ID NO: 35. In some embodiments, the immunoconjugate comprises an anti-CD79 antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 38. In some embodiments, the immunoconjugate is polatuzumab vedotin.

In some embodiments, provided is an immunoconjugate comprising the formula

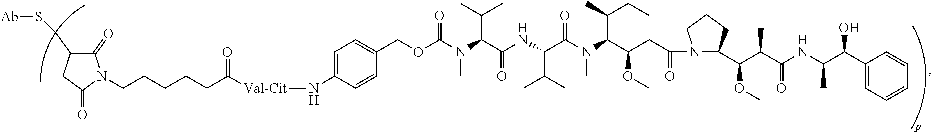

wherein Ab is an anti-CD79b antibody that comprises (i) a VH comprising the amino acid sequence of SEQ ID NO: 19 and (ii) a VL comprising the amino acid sequence of SEQ ID NO: 20, and wherein p is between 2 and 5, for use in the manufacture of a medicament for treating diffuse large B-cell lymphoma (DLBCL), e.g., relapsed/refractory DLBCL, in an individual (a human individual) in need thereof, wherein the medicament is for (e.g., formulated for) administration in combination with lenalidomide, and obinutuzumab, wherein the medicament is formulated for administration of the immunoconjugate at a dose between about 1.4 and about 1.8 mg/kg, the lenalidomide is for administration at a dose between about 10 mg and 20 mg, and the obinutuzumab is for administration at a dose of about 1000 mg. In some embodiments, the individual achieves at least stable disease (SD) (e.g., at least SD, at least partial response (PR) or a complete response (CR)) during or following the treatment with the medicament, the lenalidomide, and obinutuzumab. In some embodiments, the medicament (i.e., the medicament comprising the immunoconjugate) is for use according to a method described herein. In some embodiments, p is between 3 and 4. In some embodiments, p is 3.5. In some embodiments, p is 3.4. In some embodiments, the immunoconjugate comprises an anti-CD79b antibody comprising a heavy chain comprises the amino acid sequence of SEQ ID NO: 36, and wherein the light chain comprises the amino acid sequence of SEQ ID NO: 35. In some embodiments, the immunoconjugate comprises an anti-CD79 antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 38. In some embodiments, the immunoconjugate is polatuzumab vedotin.

In some embodiments, provided is an immunoconjugate comprising the formula

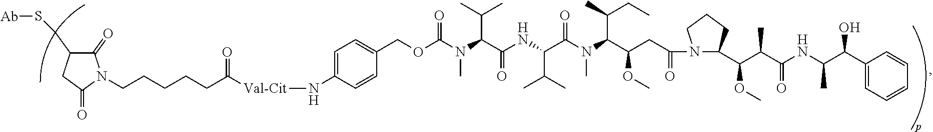

wherein Ab is an anti-CD79b antibody that comprises (i) a VH comprising the amino acid sequence of SEQ ID NO: 19 and (ii) a VL comprising the amino acid sequence of SEQ ID NO: 20, and wherein p is between 2 and 5, for use in the manufacture of a medicament for treating diffuse large B-cell lymphoma (DLBCL), e.g., relapsed/refractory DLBCL, in an individual (a human individual) in need thereof, wherein the medicament is for (e.g., formulated for) administration in combination with lenalidomide, and rituximab, wherein the medicament is formulated for administration of the immunoconjugate at a dose between about 1.4 and about 1.8 mg/kg, the lenalidomide is for administration at a dose between about 10 mg and 20 mg, and the rituximab is for administration at a dose of about 375 mg/m². In some embodiments, the individual achieves at least stable disease (SD) (e.g., at least SD, at least partial response (PR) or a complete response (CR)) during or following the treatment with the medicament, the lenalidomide, and rituximab. In some embodiments, the medicament (i.e., the medicament comprising the immunoconjugate) is for use according to a method described herein. In some embodiments, p is between 3 and 4. In some embodiments, p is 3.5. In some embodiments, p is 3.4. In some embodiments, the immunoconjugate comprises an anti-CD79b antibody comprising a heavy chain comprises the amino acid sequence of SEQ ID NO: 36, and wherein the light chain comprises the amino acid sequence of SEQ ID NO: 35. In some embodiments, the immunoconjugate comprises an anti-CD79 antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 38. In some embodiments, the immunoconjugate is polatuzumab vedotin.

### V. Immunomodulatory Agents

Immunomodulatory agents (e.g., thalidomide, lenalidomide, and pomalidomide, which are also known as “IMiDs®”) are a class of orally available antineoplastic or anticancer drugs that exhibit pleiotropic properties. For example, immunomodulatory agents stimulate NK-cell and T-cell activity and exhibit anti-angiogenic, anti-inflammatory, pro-apoptotic, and anti-proliferative effects, as well. The mechanisms of action by which immunomodulatory drugs exert their effects have not yet been fully characterized.

Lenalidomide is an exemplary immunomodulatory agent used in the methods described herein. The chemical name for lenalidomide is 344-amino-1-oxo-2,3-dihydro-1H-isoindol-2-yl)piperidine-2,6-dione, and lenalidomide has the following chemical structure:

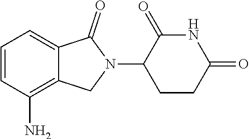 [SMILES: Nc1cccc2c1CN(C1CCC(=O)NC1=O)C2=O]

Lenalidomide (CAS Registry #191732-72-6) has the molecular formula of C₁₃H₁₃N₃O₃ and a molecular weight of 259.261 g/mol. Lenalidomide is also known as CC-5103, IMiD3 cdp. It is commercially available for therapeutic use under the trade name REVLIMID®, and is provided as 2.5 mg, 5 mg, 10 mg, 15 mg, 20 mg, and 25 mg capsules. Lenalidomide may be provided in a dose of, for example, 2.5 mg, 5 mg, 10 mg, 15 mg, 20 mg, or 25 mg.

### VI. Anti-CD20 Agents

Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1052, see Table S.

TABLE S
Properties of type I and type II anti-CD20 antibodies

| Type I anti-CD20 antibodies | Type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon | Strong cell death induction without |
| cross-linking | cross-linking |

Examples of type I anti-CD20 antibodies include e.g., rituximab, HI47 IgG3 (ECACC, hybridoma), 2C6 IgG1 (as disclosed in WO 2005/103081), 2F2 IgG1 (as disclosed and WO 2004/035607 and WO 2005/103081) and 2H7 IgG1 (as disclosed in WO 2004/056312).

In some embodiments, the anti-CD20 antibody used in a method of treatment provided herein comprises, according to numbering in Kabat et al., the CDR-H1, CDR-H2, CDR-H3, CDR-L1, CDR-L2, and CDR-L3 of rituximab. In some embodiments, the anti-CD20 antibody used in a method of treatment provided herein comprises the VH and the VL of rituximab. In some embodiments, the anti-CD20 antibody used in a method of treatment provided herein comprises the heavy chain and the light chain of rituximab. As used herein, the term “rituximab” refers to an anti-CD20 antibody having the CAS Registry Number 174722-31-7. In some embodiments, the anti-CD20 antibody used a method of treatment provided herein is rituximab. In some embodiments, the rituximab (reference antibody; example of a type I anti-CD20 antibody) is a genetically engineered chimeric human gamma 1 murine constant domain containing monoclonal antibody directed against the human CD20 antigen. However this antibody is not glycoengineered and not afucosylated and thus has an amount of fucose of at least 85%. This chimeric antibody comprises human gamma 1 constant domains and is identified by the name “C2B8” in U.S. Pat. No. 5,736,137 (Andersen, et. al.) issued on Apr. 17, 1998, assigned to IDEC Pharmaceuticals Corporation. Rituximab is approved for the treatment of patients with relapsed or refracting low-grade or follicular, CD20 positive, B-cell non-Hodgkin's lymphoma. In vitro mechanism of action studies have shown that rituximab exhibits human complement-dependent cytotoxicity (CDC) (Reff, M. E., et. al, Blood 83(2) (1994) 435-445). Additionally, it exhibits activity in assays that measure antibody-dependent cellular cytotoxicity (ADCC).

In some embodiments, the anti-CD20 antibody used in a method of treatment provided herein is an afucosylated anti-CD20 antibody.

Examples of type II anti-CD20 antibodies include e.g., humanized B-Ly1 antibody IgG1 (a chimeric humanized IgG1 antibody as disclosed in WO 2005/044859), 11B8 IgG1 (as disclosed in WO 2004/035607), and AT80 IgG1. Typically type II anti-CD20 antibodies of the IgG1 isotype show characteristic CDC properties. Type II anti-CD20 antibodies have a decreased CDC (if IgG1 isotype) compared to type I antibodies of the IgG1 isotype. In some embodiments the type II anti-CD20 antibody, e.g., a GA101 antibody, has increased antibody dependent cellular cytotoxicity (ADCC). In some embodiments, the type II anti-CD20 antibodies, more preferably an afucosylated humanized B-Ly1 antibody as described in WO 2005/044859 and WO 2007/031875.

In some embodiments, the anti-CD20 antibody used in a method of treatment provided herein is GA101 antibody. In some embodiments, the GA101 antibody as used herein refers to any one of the following antibodies that bind human CD20: (1) an antibody comprising an HVR-H1 comprising the amino acid sequence of SEQ ID NO:5, an HVR-H2 comprising the amino acid sequence of SEQ ID NO:6, an HVR-H3 comprising the amino acid sequence of SEQ ID NO:7, an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10; (2) an antibody comprising a VH domain comprising the amino acid sequence of SEQ ID NO:11 and a VL domain comprising the amino acid sequence of SEQ ID NO:12, (3) an antibody comprising an amino acid sequence of SEQ ID NO:13 and an amino acid sequence of SEQ ID NO: 14; (4) an antibody known as obinutuzumab, or (5) an antibody that comprises an amino acid sequence that has at least 95%, 96%, 97%, 98% or 99% sequence identity with amino acid sequence of SEQ ID NO:13 and that comprises an amino acid sequence that has at least 95%, 96%, 97%, 98% or 99% sequence identity with an amino acid sequence of SEQ ID NO: 14. In one embodiment, the GA101 antibody is an IgG1 isotype antibody.

In some embodiments, the anti-CD20 antibody used in a method of treatment provided herein is a humanized B-Ly1 antibody. In some embodiments, the humanized B-Ly1 antibody refers to humanized B-Ly1 antibody as disclosed in WO 2005/044859 and WO 2007/031875, which were obtained from the murine monoclonal anti-CD20 antibody B-Ly1 (variable region of the murine heavy chain (VH): SEQ ID NO: 3; variable region of the murine light chain (VL): SEQ ID NO: 4—see Poppema, S. and Visser, L., Biotest Bulletin 3 (1987) 131-139) by chimerization with a human constant domain from IgG1 and following humanization (see WO 2005/044859 and WO 2007/031875). The humanized B-Ly1 antibodies are disclosed in detail in WO 2005/044859 and WO 2007/031875.

In some embodiments, the humanized B-Ly1 antibody has variable region of the heavy chain (VH) selected from group of SEQ ID NO:15-16 and 40-54 (corresponding to B-HH2 to B-HH9 and B-HL8 to B-HL17 of WO 2005/044859 and WO 2007/031875). In some embodiments, the variable domain is selected from the group consisting of SEQ ID NO: 15, 16, 42, 44, 46, 48 and 50 (corresponding to B-HH2, BHH-3, B-HH6, B-HH8, B-HL8, B-HL11 and B-HL13 of WO 2005/044859 and WO 2007/031875). In some embodiments, the humanized B-Ly1 antibody has variable region of the light chain (VL) of SEQ ID NO:55 (corresponding to B-KV1 of WO 2005/044859 and WO 2007/031875). In some embodiments, the humanized B-Ly1 antibody has a variable region of the heavy chain (VH) of SEQ ID NO:42 (corresponding to B-HH6 of WO 2005/044859 and WO 2007/031875) and a variable region of the light chain (VL) of SEQ ID NO:55 (corresponding to B-KV1 of WO 2005/044859 and WO 2007/031875). In some embodiments, the humanized B-Ly1 antibody is an IgG1 antibody. Such afucosylated humanized B-Ly1 antibodies are glycoengineered (GE) in the Fc region according to the procedures described in WO 2005/044859, WO 2004/065540, WO 2007/031875, Umana, P. et al., Nature Biotechnol. 17 (1999) 176-180 and WO 99/154342. In some embodiments, the afucosylated glyco-engineered humanized B-Ly1 is B-HH6-B-KV1 GE. In some embodiments, the anti-CD20 antibody is obinutuzumab (recommended INN, WHO Drug Information, Vol. 26, No. 4, 2012, p. 453). As used herein, obinutuzumab is synonymous for GA101 or R05072759. It is commercially available for therapeutic use under the trade name GAZYVA®, and is provided as a 1000 mg/40 mL (25 mg/mL) single-dose vial. This replaces all previous versions (e.g., Vol. 25, No. 1, 2011, p. 75-76), and is formerly known as afutuzumab (recommended INN, WHO Drug Information, Vol. 23, No. 2, 2009, p. 176; Vol. 22, No. 2, 2008, p. 124). In some embodiments, the humanized B-Ly1 antibody is an antibody comprising a heavy chain comprising the amino acid sequence of SEQ ID NO:17 and a light chain comprising the amino acid sequence of SEQ ID NO:18, or an antigen-binding fragment thereof such antibody. In some embodiments, the humanized B-Ly1 antibody comprises a heavy chain variable region comprising the three heavy chain CDRs of SEQ ID NO:17 and a light chain variable region comprising the three light chain CDRs of SEQ ID NO:18.

In some embodiments, the humanized B-Ly1 antibody is an afucosylated glyco-engineered humanized B-Ly1. Such glycoengineered humanized B-Ly1 antibodies have an altered pattern of glycosylation in the Fc region, preferably having a reduced level of fucose residues. In some embodiments, the amount of fucose is about 60% or less of the total amount of oligosaccharides at Asn297 (in one embodiment the amount of fucose is between about 40% and about 60%, in another embodiment the amount of fucose is about 50% or less, and in still another embodiment the amount of fucose is about 30% or less). In some embodiments, the oligosaccharides of the Fc region are bisected. These glycoengineered humanized B-Ly1 antibodies have an increased ADCC.

The “ratio of the binding capacities to CD20 on Raji cells (ATCC-No. CCL-86) of an anti-CD20 antibodies compared to rituximab” is determined by direct immunofluorescence measurement (the mean fluorescence intensities (MFI) is measured) using said anti-CD20 antibody conjugated with Cy5 and rituximab conjugated with Cy5 in a FACSArray (Becton Dickinson) with Raji cells (ATCC-No. CCL-86), as described in Example No. 2, and calculated as follows:

$$Ratio of the binding capacities to CD 20 on Raji cells ( ATCC - No . CCL - 86 ) = \frac{M F I ( Cy 5 - anti - CD 20 antibody )}{M F I ( Cy 5 - rituximab )} \times \frac{Cy 5 - labeling ratio ( Cy 5 - rituximab )}{Cy 5 - labeling ratio ( Cy 5 - anti - CD 20 antibody )}$$

MFI is the mean fluorescent intensity. The “Cy5-labeling ratio” as used herein means the number of Cy5-label molecules per molecule antibody.

Typically said type II anti-CD20 antibody has a ratio of the binding capacities to CD20 on Raji cells (ATCC-No. CCL-86) of said second anti-CD20 antibody compared to rituximab of 0.3 to 0.6, and in one embodiment, 0.35 to 0.55, and in yet another embodiment, 0.4 to 0.5.

By “antibody having increased antibody dependent cellular cytotoxicity (ADCC)”, it is meant an antibody, as that term is defined herein, having increased ADCC as determined by any suitable method known to those of ordinary skill in the art.

An exemplary accepted in vitro ADCC assay is described below:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10⁶ cells/ml in RPMI cell culture medium;
   ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10′ cells/ml;
   iii) 100 microliters of the final target cell suspension above are transferred to each well of a 96-well microtiter plate;
   iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (VN) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector:target cell ratio of 25:1 and the plates are placed in an incubator under 5% CO2 atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point V above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. In one embodiment, the increase in ADCC is relative to the ADCC, measured with the above assay, mediated by the same antibody, produced by the same type of host cells, using the same standard production, purification, formulation and storage methods, which are known to those skilled in the art, except that the comparator antibody (lacking increased ADCC) has not been produced by host cells engineered to overexpress GnTIII and/or engineered to have reduced expression from the fucosyltransferase 8 (FUT8) gene (e.g., including, engineered for FUT8 knock out).

In some embodiments, the “increased ADCC” can be obtained by, for example, mutating and/or glycoengineering of said antibodies. In some embodiments, the anti-CD20 antibody is glycoengineered to have a biantennary oligosaccharide attached to the Fc region of the antibody that is bisected by GlcNAc. In some embodiments, the anti-CD20 antibody is glycoengineered to lack fucose on the carbohydrate attached to the Fc region by expressing the antibody in a host cell that is deficient in protein fucosylation (e.g., Lec13 CHO cells or cells having an alpha-1,6-fucosyltransferase gene (FUT8) deleted or the FUT gene expression knocked down). In some embodiments, the anti-CD20 antibody sequence has been engineered in its Fc region to enhance ADCC. In some embodiments, such engineered anti-CD20 antibody variant comprises an Fc region with one or more amino acid substitutions at positions 298, 333, and/or 334 of the Fc region (EU numbering of residues)).

In some embodiments, the term “complement-dependent cytotoxicity (CDC)” refers to lysis of human cancer target cells by the antibody according to the invention in the presence of complement. CDC can be measured by the treatment of a preparation of CD20 expressing cells with an anti-CD20 antibody according to the invention in the presence of complement. CDC is found if the antibody induces at a concentration of 100 nM the lysis (cell death) of 20% or more of the tumor cells after 4 hours. In some embodiments, the assay is performed with ⁵¹Cr or Eu labeled tumor cells and measurement of released ⁵¹Cr or Eu. Controls include the incubation of the tumor target cells with complement but without the antibody.

In some embodiments, the anti-CD20 antibody is a monoclonal antibody, e.g., a human antibody. In some embodiments, the anti-CD20 antibody is an antibody fragment, e.g., a Fv, Fab, Fab′, scFv, diabody, or F(ab′)₂ fragment. In some embodiments, the anti-CD20 antibody is a substantially full length antibody, e.g., an IgG1 antibody, IgG2a antibody or other antibody class or isotype as defined herein.

### VII. Antibodies

In some embodiments, an antibody (e.g., an anti-CD79b antibody or an anti-CD20 antibody) used in a method of treatment provided herein may incorporate any of the features, singly or in combination, as described in below.

A. Antibody Affinity

In certain embodiments, an antibody (e.g., an anti-CD79b antibody or an anti-CD20 antibody) used in a method of treatment provided herein has a dissociation constant (Kd) of ≤1 μM, ≤100 nM, ≤50 nM, ≤10 nM, ≤5 nM, ≤1 nM, ≤0.1 nM, ≤0.01 nM, or ≤0.001 nM, and optionally is ≥10⁻¹³ M. (e.g., 10⁻⁸M or less, e.g., from 10⁻⁸M to 10³ M, e.g., from 10⁻⁹ M to ≥10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, NJ) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 l/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

B. Antibody Fragments

In certain embodiments, an antibody (e.g., an anti-CD79b antibody or an anti-CD20 antibody) used in a method of treatment provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

C. Chimeric and Humanized Antibodies

In certain embodiments, an antibody (e.g., an anti-CD79b antibody or an anti-CD20 antibody) used in a method of treatment provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

D. Human Antibodies

In certain embodiments, an antibody (e.g., an anti-CD79b antibody or an anti-CD20 antibody) used in a method of treatment provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

E. Library-Derived Antibodies

In some embodiments, an antibody (e.g., an anti-CD79b antibody or an anti-CD20 antibody) used in a method of treatment provided herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

F. Multispecific Antibodies

In certain embodiments, an antibody (e.g., an anti-CD79b antibody or an anti-CD20 antibody) used in a method of treatment provided herein is a multispecific antibody, e.g., a bispecific antibody. Multispecific antibodies are monoclonal antibodies that have binding specificities for at least two different sites. In certain embodiments, one of the binding specificities is for one antigen (e.g., CD79b or CD20) and the other is for any other antigen. In certain embodiments, one of the binding specificities is for one antigen (e.g., CD79b or CD20) and the other is for CD3. See, e.g., U.S. Pat. No. 5,821,337. In certain embodiments, bispecific antibodies may bind to two different epitopes of an single antigen (e.g., CD79b or CD20). Bispecific antibodies may also be used to localize cytotoxic agents to cells which express the antigen (e.g., CD79b or CD20). Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to CD79b as well as another, different antigen (see, US 2008/0069820, for example).

G. Antibody Variants

In certain embodiments, amino acid sequence variants of an antibody (e.g., an anti-CD79b antibody or an anti-CD20 antibody) used in a method of treatment provided herein are contemplated. For example, it may be desirable to improve the binding affinity and/or other biological properties of the anti-CD79b antibody or anti-CD20 antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

(i) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table T under the heading of “preferred substitutions.” More substantial changes are provided in Table T under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE T

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Len, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

(ii) Glycosylation Variants

In certain embodiments, an antibody (e.g., an anti-CD79b antibody or an anti-CD20 antibody) used in a method of treatment provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

(iii) Fc Variants

In certain embodiments, one or more amino acid modifications may be introduced into the Fc region of an antibody (e.g., an anti-CD79b antibody or an anti-CD20 antibody) used in a method of treatment provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C₃c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

(iv) Cysteine Engineered Antibody Variants

In certain embodiments, it may be desirable to create cysteine engineered antibodies, e.g., “thioMAbs,” in which one or more residues of an anti-CD79b antibody or an anti-CD20 antibody used in a method of treatment provided herein are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

(v) Antibody Derivatives

In certain embodiments, an antibody (e.g., an anti-CD79b antibody or an anti-CD20 antibody) used in a method of treatment provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

H. Recombinant Methods and Compositions

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

I. Assays

An antibody (e.g., an anti-CD79b antibody or an anti-CD20 antibody) used in a method of treatment provided herein may be identified, screened for, or characterized for physical/chemical properties and/or biological activities by various assays known in the art.

In one aspect, an antibody (e.g., an anti-CD79b antibody or an anti-CD20 antibody) used in a method of treatment provided herein is tested for its antigen binding activity, e.g., by known methods such as ELISA, BIACore®, FACS, or Western blot.

In another aspect, competition assays may be used to identify an antibody that competes with any of the antibodies described herein for binding to the target antigen. In certain embodiments, such a competing antibody binds to the same epitope (e.g., a linear or a conformational epitope) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized antigen is incubated in a solution comprising a first labeled antibody that binds to antigen (e.g., any of the antibodies described herein) and a second unlabeled antibody that is being tested for its ability to compete with the first antibody for binding to antigen. The second antibody may be present in a hybridoma supernatant. As a control, immobilized antigen is incubated in a solution comprising the first labeled antibody but not the second unlabeled antibody. After incubation under conditions permissive for binding of the first antibody to antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to antigen. See Harlow and Lane (1988) Antibodies: A Laboratory Manual ch. 14 (Cold Spring Harbor Laboratory, Cold Spring Harbor, NY).

### VIII. Pharmaceutical Formulations

Pharmaceutical formulations of any of the agents described herein (e.g., anti-CD79b immunoconjugates, anti-CD20 agents, and immunomodulatory agents) for use in any of the methods as described herein are prepared by mixing such agent(s) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g., films, or microcapsules.

The formulations to be used for in vivo administration are generally sterile. Sterility may be readily accomplished, e.g., by filtration through sterile filtration membranes.

Additional details regarding pharmaceutical formulations comprising an anti-CD79 immunoconjugate are provided in WO 2009/099728 the contents of which are expressly incorporated by reference herein in their entirety.

### IX. Kits and Articles of Manufacture

In another embodiment, an article of manufacture or a kit is provided comprising an anti-CD79b immunoconjugate (such as described herein) and at least one additional agent. In some embodiments the at least one additional agent is an immunomodulatory agent (such as lenalidomide) and an anti-CD20 antibody (such as obinutuzumab or rituximab). In some embodiments, the article of manufacture or kit further comprises a package insert comprising instructions for using the anti-CD79b immunoconjugate in conjunction at least one additional agent, such as an immunomodulatory agent (e.g., lenalidomide) and an anti-CD20 antibody (e.g., obinutuzumab or rituximab) to treat or delay progression of a B-cell proliferative disorder (e.g., DLBCL, such as relapsed/refractory DLBCL) in an individual. Any of the anti-CD79b immunoconjugates, immunomodulatory agents, and/or anti-CD20 antibodies, and optionally one or more additional anti-cancer agents, known in the art or described herein may be included in the article of manufacture or kits. In some embodiments, the kit comprises an immunoconjugate comprising the formula

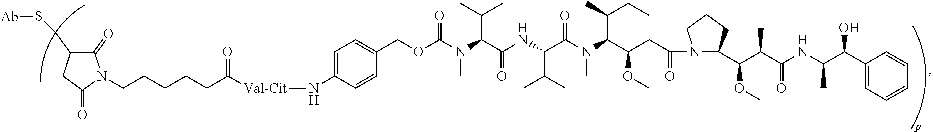

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8. In some embodiments, the kit comprises an immunoconjugate comprising the formula

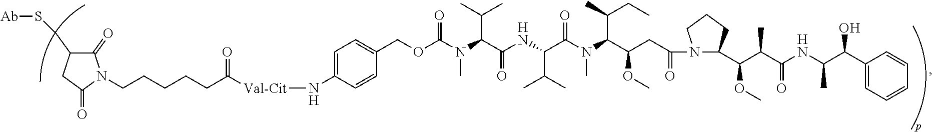

wherein Ab is an anti-CD79b antibody that comprises (i) a heavy chain comprising a VH that comprises the amino acid sequence of SEQ ID NO: 19 and (ii) a light chain comprising a VL that comprises the amino acid sequence of SEQ ID NO: 20, and wherein p is between 2 and 5. In some embodiments, p is between 3 and 4, e.g., 3.4 or 3.5. In some embodiments, the immunoconjugate comprises an anti-CD79b antibody comprising a heavy chain comprising the amino acid sequence of SEQ ID NO: 36, and a light chain comprising the amino acid sequence of SEQ ID NO: 35. In certain embodiments, the anti-CD79b immunoconjugate comprises the structure of Ab-MC-vc-PAB-MMAE. In some embodiments, the anti-CD79b immunoconjugate is polatuzumab vedotin (CAS Number 1313206-42-6). In some embodiments, the at least one additional agent is an immunomodulatory agent (such as lenalidomide) and an anti-CD20 antibody (such as obinutuzumab or rituximab). In some embodiments, the kit is for use in the treatment of DLBCL, e.g., R/R DLBCL, in an individual, such as a human (e.g., a human having one or more characteristics described herein) according to a method provided herein.

In some embodiments, the anti-CD79 immunoconjugate, the immunomodulatory agent (e.g., lenalidomide) and the anti-CD20 antibody (such as obinutuzumab or rituximab) are in the same container or in separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation, and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

TABLE U
Amino Acid Sequences

| NAME | SEQUENCE | SEQ ID NO |
|---|---|---|
| Human CD79b | RFIARKRGFT VKMHCYMNSA SGNVSWLWKQ EMDENPQQLK | 1 |
| precursor; Acc. No. | LEKGRMEESQ NESLATLTIQ GIRFEDNGIY FCQQKCNNTS | |
| NP_000617.1; signal | EVYQGCGTEL RVMGFSTLAQ LKQRNTLKDG IIMIQTLLII | |
| sequence = amino | LFIIVPIFLL LDKDDSKAGM EEDHTYEGLD IDQTATYEDI | |
| acids 1 to 28 | VTLRTGEVKW SVGEHPGQE | |
| Human mature CD79b, | AR SEDRYRNPKG SACSRIWQSP RFIARKRGFT VKMHCYMNSA | 2 |
| without signal | SGNVSWLWKQ EMDENPQQLK LEKGRMEESQ NESLATLTIQ | |
| sequence; amino | GIRFEDNGIY FCQQKCNNTS EVYQGCGTEL RVMGESTLAQ | |
| acids 29 to 229 | LKQRNTLKDG IIMIQTLLII LFIIVPIFLL LDKDDSKAGM | |
| | EEDHTYEGLD IDQTATYEDI VTLRTGEVKW SVGEHPGQE | |
| VH of mMAb anti- | Gly Pro Glu Leu Val Lys Pro Gly Ala Ser Val | 3 |
| CD20 antibody B-Ly1 | Lys Ile Ser Cys Lys Ala Ser Gly Tyr Ala Phe | |
| | Ser Tyr Ser Trp Met Asn Trp Val Lys Leu Arg | |
| | Pro Gly Gln Gly Leu Glu Trp Ile Gly Arg Ile | |
| | Phe Pro Gly Asp Gly Asp Thr Asp Tyr Asn Gly | |
| | Lys Phe Lys Gly Lys Ala Thr Leu Thr Ala Asp | |
| | Lys Ser Ser Asn Thr Ala Tyr Met Gln Leu Thr | |
| | Ser Leu Thr Ser Val Asp Ser Ala Val Tyr Leu | |
| | Cys Ala Arg Asn Val Phe Asp Gly Tyr Trp Leu | |
| | Val Tyr Trp Gly Gln Gly Thr Leu Val Thr Val | |
| | Ser Ala | |
| VL of mMAb anti- | Asn Pro Val Thr Leu Gly Thr Ser Ala Ser Ile | 4 |
| CD20 antibody B-Ly1 | Ser Cys Arg Ser Ser Lys Ser Leu Leu His Ser | |
| | Asn Gly Ile Thr Tyr Leu Tyr Trp Tyr Leu Gln | |
| | Lys Pro Gly Gln Ser Pro Gln Leu Leu Ile Tyr | |
| | Gln Met Ser Asn Leu Val Ser Gly Val Pro Asp | |
| | Arg Phe Ser Ser Ser Gly Ser Gly Thr Asp Phe | |
| | Thr Leu Arg Ile Ser Arg Val Glu Ala Glu Asp | |
| | Val Gly Val Tyr Tyr Cys Ala Gln Asn Leu Glu | |
| | Leu Pro Tyr Thr Phe Gly Gly Gly Thr Lys Leu | |
| | Glu Ile Lys Arg | |
| GA101 HVR-H1 | Gly Tyr Ala Phe Ser Tyr | 5 |
| GA101 HVR-H2 | Phe Pro Gly Asp Gly Asp Thr Asp | 6 |
| GA101 HVR-H3 | Asn Val Phe Asp Gly Tyr Trp Leu Val Tyr | 7 |
| GA101 HVR-L1 | Arg Ser Ser Lys Ser Leu Leu His Ser Asn Gly | 8 |
| | Ile Thr Tyr Leu Tyr | |
| GA101 HVR-L2 | Gln Met Ser Asn Leu Val Ser | 9 |
| GA101 HVR-L3 | Ala Gln Asn Leu Glu Leu Pro Tyr Thr | 10 |
| GA101 VH | Gln Val Gln Leu Val Gln Ser Gly Ala Glu Val | 11 |
| | Lys Lys Pro Gly Ser Ser Val Lys Val Ser Cys | |
| | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Thr Val Ser Ser | |
| GA101 VL | Asp Ile Val Met Thr Gln Thr Pro Leu Ser Leu | 12 |
| | Pro Val Thr Pro Gly Glu Pro Ala Ser Ile Ser | |
| | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys Arg Thr Val | |
| GA101 Heavy Chain | Gln Val Gln Leu Val Gln Ser Gly Ala Glu Val | 13 |
| | Lys Lys Pro Gly Ser Ser Val Lys Val Ser Cys | |
| | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser Ala Ser | |
| | Thr Lys Gly Pro Ser Val Phe Pro Leu Ala Pro | |
| | Ser Ser Lys Ser Thr Ser Gly Gly Thr Ala Ala | |
| | Leu Gly Cys Leu Val Lys Asp Tyr Phe Pro Glu | |
| | Pro Val Thr Val Ser Trp Asn Ser Gly Ala Leu | |
| | Thr Ser Gly Val His Thr Phe Pro Ala Val Leu | |
| | Gln Ser Ser Gly Leu Tyr Ser Leu Ser Ser Val | |
| | Val Thr Val Pro Ser Ser Ser Leu Gly Thr Gln | |
| | Thr Tyr Ile Cys Asn Val Asn His Lys Pro Ser | |
| | Asn Thr Lys Val Asp Lys Lys Val Glu Pro Lys | |
| | Ser Cys Asp Lys Thr His Thr Cys Pro Pro Cys | |
| | Pro Ala Pro Glu Leu Leu Gly Gly Pro Ser Val | |
| | Phe Leu Phe Pro Pro Lys Pro Lys Asp Thr Leu | |
| | Met Ile Ser Arg Thr Pro Glu Val Thr Cys Val | |
| | Val Val Asp Val Ser His Glu Asp Pro Glu Val | |
| | Lys Phe Asn Trp Tyr Val Asp Gly Val Glu Val | |
| | His Asn Ala Lys Thr Lys Pro Arg Glu Glu Gln | |
| | Tyr Asn Ser Thr Tyr Arg Val Val Ser Val Leu | |
| | Thr Val Leu His Gln Asp Trp Leu Asn Gly Lys | |
| | Glu Tyr Lys Cys Lys Val Ser Asn Lys Ala Leu | |
| | Pro Ala Pro Ile Glu Lys Thr Ile Ser Lys Ala | |
| | Lys Gly Gln Pro Arg Glu Pro Gln Val Tyr Thr | |
| | Leu Pro Pro Ser Arg Asp Glu Leu Thr Lys Asn | |
| | Gln Val Ser Leu Thr Cys Leu Val Lys Gly Phe | |
| | Tyr Pro Ser Asp Ile Ala Val Glu Trp Glu Ser | |
| | Asn Gly Gln Pro Glu Asn Asn Tyr Lys Thr Thr | |
| | Pro Pro Val Leu Asp Ser Asp Gly Ser Phe Phe | |
| | Leu Tyr Ser Lys Leu Thr Val Asp Lys Ser Arg | |
| | Trp Gln Gln Gly Asn Val Phe Ser Cys Ser Val | |
| | Met His Glu Ala Leu His Asn His Tyr Thr Gln | |
| | Lys Ser Leu Ser Leu Ser Pro Gly | |
| GA101 Light Chain | Asp Ile Val Met Thr Gln Thr Pro Leu Ser Leu | 14 |
| | Pro Val Thr Pro Gly Glu Pro Ala Ser Ile Ser | |
| | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys Arg Thr Val Ala Ala Pro Ser Val Phe | |
| | Ile Phe Pro Pro Ser Asp Glu Gln Leu Lys Ser | |
| | Gly Thr Ala Ser Val Val Cys Leu Leu Asn Asn | |
| | Phe Tyr Pro Arg Glu Ala Lys Val Gln Trp Lys | |
| | Val Asp Asn Ala Leu Gln Ser Gly Asn Ser Gln | |
| | Glu Ser Val Thr Glu Gln Asp Ser Lys Asp Ser | |
| | Thr Tyr Ser Leu Ser Ser Thr Leu Thr Leu Ser | |
| | Lys Ala Asp Tyr Glu Lys His Lys Val Tyr Ala | |
| | Cys Glu Val Thr His Gln Gly Leu Ser Ser Pro | |
| | Val Thr Lys Ser Phe Asn Arg Gly Glu Cys | |
| VH of humanized B- | Gln Val Gln Leu Val Gln Ser Gly Ala Glu Val | 15 |
| Ly1 antibody (B- | Lys Lys Pro Gly Ser Ser Val Lys Val Ser Cys | |
| HH2) | Lys Ala Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | |
| Ly1 antibody (B- | Lys Lys Pro Gly Ser Ser Val Lys Val Ser Cys | |
| HH3) | Lys Ala Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | 16 |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Leu Cys Ala Arg Asn | |
| | Val Phe Asp Gly Tyr Trp Leu Val Tyr Trp Gly | |
| | Gln Gly Thr Leu Val Thr Val Ser Ser | |
| humanized B-Ly1 | QVQLVQSGAE VKKPGSSVKV SCKASGYAFS YSWINWVRQA | 17 |
| Heavy Chain | PGQGLEWMGR IFPGDGDTDY NGKFKGRVTI TADKSTSTAY | |
| | MELSSLRSED TAVYYCARNV FDGYWLVYWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPELLGGP | |
| | SVFLFPPKPK DTLMISRTPE VTCVVVDVSH EDPEVKENWY | |
| | VDGVEVHNAK TKPREEQYNS TYRVVSVLIV LHQDWLNGKE | |
| | YKCKVSNKAL PAPIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| humanized B-Ly1 | DIVMTQTPLS LPVTPGEPAS ISCRSSKSLL HSNGITYLYW | 18 |
| Light Chain | YLQKPGQSPQ LLIYQMSNLV SGVPDRESGS GSGTDFTLKI | |
| | SRVEAEDVGV YYCAQNLELP YTFGGGTKVE IKRTVAAPSV | |
| | FIFPPSDEQL KSGTASVVCL LNNFYPREAK VQWKVDNALQ | |
| | SGNSQESVTE QDSKDSTYSL SSTLTLSKAD YEKHKVYACE | |
| | VTHQGLSSPV TKSFNRGEC | |
| huMA79bv28 heavy | EVQLVESGGG LVQPGGSLRL SCAASGYTFS SYWIEWVRQA | |
| chain variable | PGKGLEWIGE ILPGGGDTNY NEIFKGRATF SADTSKNTAY | 19 |
| region | LQMNSLRAED TAVYYCTRRV PIRLDYWGQG TLVTVSS | |
| huMA79bv28 light | DIQLTQSPSS LSASVGDRVT ITCKASQSVD YEGDSFLNWY | 20 |
| chain variable | QQKPGKAPKL LIYAASNLES GVPSRESGSG SGTDFTLTIS | |
| region | SLQPEDFATY YCQQSNEDPL TFGQGTKVEI KR | |
| huMA79bv28 HVR H1 | GYTFSSYWIE | 21 |
| huMA79bv28 HVR H2 | GEILPGGGDTNYNEIFKG | 22 |
| huMA79bv28 HVR H3 | TRRVPIRLDY | 23 |
| huMA79bv28 HVR L1 | KASQSVDYEGDSFLN | 24 |
| huMA79bv28 HVR L2 | AASNLES | 25 |
| huMA79bv28 HVR L3 | QQSNEDPLT | 26 |
| huMA79bv28 heavy | EVQLVESGGGLVQPGGSLRLSCAAS | 27 |
| chain (HC) | | |
| framework region | | |
| (FR) 1 | | |
| huMA79bv28 HC FR2 | WVRQAPGKGLEWI | 28 |
| huMA79bv28 HC FR3 | RATFSADTSKNTAYLQMNSLRAEDTAVYYC | 29 |
| huMA79bv28 HC FR4 | WGQGTLVTVSS | 30 |
| huMA79bv28 light | DIQLTQSPSSLSASVGDRVTITC | 31 |
| chain (LC) FR1 | | |
| huMA79bv28 LC FR2 | WYQQKPGKAPKLLIY | 32 |
| huMA79bv28 LC FR3 | GVPSRFSGSGSGTDFTLTISSLQPEDFATYYC | 33 |
| huMA79bv28 LC FR4 | FGQGTKVEIKR | 34 |
| huMA79bv28 light | DIQLTQSPSS LSASVGDRVT ITCKASQSVD YEGDSFLNWY | 35 |
| chain (Igκ) | QQKPGKAPKL LIYAASNLES GVPSRFSGSG SGTDFTLTIS | |
| | SLQPEDFATY YCQQSNEDPL TFGQGTKVEI KRTVAAPSVE | |
| | IFPPSDEQLK SGTASVVCLL NNFYPREAKV QWKVDNALQS | |
| | GNSQESVTEQ DSKDSTYSLS STLTLSKADY EKHKVYACEV | |
| | THQGLSSPVT KSFNRGEC | |
| huMA79bv28 heavy | EVQLVESGGG LVQPGGSLRL SCAASGYTFS SYWIEWVRQA | 36 |
| chain (IgG1) | PGKGLEWIGE ILPGGGDTNY NEIFKGRATF SADTSKNTAY | |
| | LQMNSLRAED TAVYYCTRRV PIRLDYWGQG TLVTVSSAST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKENWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDS | |
| | DGSFFLYSKLT VDKSRWQQG NVFSCSVMHE ALHNHYTQKS | |
| | LSLSPG | |
| huMA79bv28 A118C | EVQLVESGGG LVQPGGSLRL SCAASGYTFS SYWIEWVRQA | 37 |
| cysteine engineered | PGKGLEWIGE ILPGGGDTNY NEIFKGRATF SADTSKNTAY | |
| heavy chain (IgG1) | LQMNSLRAED TAVYYCTRRV PIRLDYWGQG TLVTVSSCST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKENWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDS | |
| | DGSFFLYSKL TVDKSRWQQG NVFSCSVMHE ALHNHYTQKS | |
| | LSLSPG | |
| huMA79bv28 V205C | DIQLTQSPSS LSASVGDRVT ITCKASQSVD YEGDSELNWY | 38 |
| cysteine engineered | QQKPGKAPKL LIYAASNLES GVPSRFSGSG SGTDFTLTIS | |
| light chain (Igκ) | SLQPEDFATY YCQQSNEDPL TFGQGTKVEI KRIVAAPSVE | |
| | IFPPSDEQLK SGTASVVCLL NNFYPREAKV QWKVDNALQS | |
| | GNSQESVTEQ DSKDSTYSLS STLTLSKADY EKHKVYACEV | |
| | THQGLSSPCT KSENRGEC | |
| huMA79bv28 S400C | EVQLVESGGG LVQPGGSLRL SCAASGYTFS SYWIEWVRQA | 39 |
| cysteine engineered | PGKGLEWIGE ILPGGGDTNY NEIFKGRATF SADTSKNTAY | |
| heavy chain (IgG1) | LQMNSLRAED TAVYYCTRRV PIRLDYWGQG TLVTVSSAST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKENWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDC | |
| | DGSFFLYSKL TVDKSRWQQG NVFSCSVMHE ALHNHYTQKS | |
| | LSLSPGK | |
| VH of humanized B- | Gln Val Gln Leu Val Gln Ser Gly Ala Glu Val | 40 |
| Ly1 antibody (B- | Lys Lys Pro Gly Ala Ser Val Lys Val Ser Cys | |
| HH4) | Lys Val Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 41 |
| Ly1 antibody (B- | Lys Lys Pro Gly Ser Ser Val Lys Val Ser Cys | |
| HH5) | Lys Ala Ser Gly Tyr Ala Phe Ser Tyr Ser Trp | |
| | Met Ser Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 42 |
| Ly1 antibody (B- | Lys Lys Pro Gly Ser Ser Val Lys Val Ser Cys | |
| HH6) | Lys Ala Ser Gly Tyr Ala Phe Ser Tyr Ser Trp | |
| | Ile Asn Trp Val Arg Gln Ala Pro Gly iln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Thr Val Ser Ser | |
| VH of humanized B- | Gln Val Gln Leu Val Gln Ser Gly Ala Glu Val | 43 |
| Ly1 antibody (B- | Lys Lys Pro Gly Ser Ser Val Lys Val Ser Cys | |
| HH7) | Lys Ala Ser Gly Tyr Ala Phe Ser Tyr Ser Trp | |
| | Ile Ser Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 44 |
| Ly1 antibody (B- | Lys Lys Pro Gly Ala Ser Val Lys Val Ser Cys | |
| HH8) | Lys Ala Ser Gly Tyr Thr Phe Thr Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 45 |
| Ly1 antibody (B- | Lys Lys Pro Gly Ala Ser Val Lys Val Ser Cys | |
| HH9) | Lys Ala Ser Gly Tyr Thr Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 46 |
| Ly1 antibody (B- | Val Lys Pro Gly Gly Ser Leu Arg Leu Ser Cys | |
| HL8) | Ala Ala Ser Gly Phe Thr Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 47 |
| Ly1 antibody (B- | Val Lys Pro Gly Gly Ser Leu Arg Leu Ser Cys | |
| HL10) | Ala Ala Ser Gly Phe Ala Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Glu Ser Gly Gly Gly Leu | 48 |
| Ly1 antibody (B- | Val Lys Pro Gly Gly Ser Leu Arg Leu Ser Cys | |
| HL11) | Ala Ala Ser Gly Phe Thr Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Ala Gly Leu | 49 |
| Ly1 antibody (B- | Val Lys Pro Gly Gly Ser Leu Arg Leu Ser Cys | |
| HL12) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Val | 50 |
| Ly1 antibody (B- | Val Lys Pro Gly Gly Ser Leu Arg Leu Ser Cys | |
| HL13) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 51 |
| Ly1 antibody (B- | Lys Lys Pro Gly Gly Ser Leu Arg Leu Ser Cys | |
| HL14) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 52 |
| Ly1 antibody (B- | Val Lys Pro Gly Ser Ser Leu Arg Leu Ser Cys | |
| HL15) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 53 |
| Ly1 antibody (B- | Val Lys Pro Gly Gly Ser Leu Arg Val Ser Cys | |
| HL16) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 54 |
| Ly1 antibody (B- | Val Lys Pro Gly Gly Ser Leu Arg Leu Ser Cys | |
| HL17) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VL of humanized B- | Asp Ile Val Met Thr Gln Thr Pro Leu Ser Leu | 55 |
| Ly1 antibody (B- | Pro Val Thr Pro Gly Glu Pro Ala Ser Ile Ser | |
| KVI) | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys Arg Thr Val | |

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The following are examples of methods and compositions of the disclosure. It is understood that various other embodiments may be practiced, given the general description provided above.

### Example 1: A Phase Ib/II Study of an Anti-CD79b Immunoconjugate in Combination with an Anti-CD20 Antibody and an Immunomodulatory Agent in Relapsed or Refractory Diffuse Large B-Cell Lymphoma (DLBCL)

This Example describes a Phase Ib/II study evaluating the safety and efficacy of an anti-CD20 antibody (rituximab; also referred to herein as “R”) in combination with an anti-CD79b immunoconjugate (polatuzumab vedotin; also referred to herein as “Pola”) and an immunomodulatory agent (lenalidomide; also referred to herein as “Len”) in patients with relapsed or refractory diffuse large B-cell lymphoma (R/R DLBCL).

### I. Study Objectives and Endpoints

This study evaluated the safety, efficacy, and pharmacokinetics of induction treatment comprising rituximab in combination with polatuzumab vedotin and lenalidomide (R+Pola+Len) in patients with R/R DLBCL, followed by post-induction treatment with rituximab plus lenalidomide (R+Len; referred to as consolidation) in patients with DLBCL who achieve a complete response (CR) or partial response (PR) at the end of induction (EOI).

A. Safety Objectives

The safety objectives for this study were as follows:
To determine the recommended Phase II dose (RP2D) of lenalidomide when given in combination with a fixed dose of polatuzumab vedotin and rituximab on the basis of the following endpoint:
   Incidence of dose-limiting toxicities (DLTs) during the first cycle of study treatment.
To evaluate the safety and tolerability of R+Pola+Len on the basis of the following endpoints:
   Nature, frequency, severity, and timing of adverse events, including DLTs.
   Changes in vital signs, electrocardiograms (ECGs), and clinical laboratory results during and following study treatment administration.

B. Primary and Secondary Efficacy Objectives

The primary efficacy objective for this study was to evaluate the efficacy of induction treatment with R+Pola+Len in R/R DLBCL on the basis of the following endpoint:
CR at EOI, as determined by the IRC on the basis of PET-CT scans.

The secondary efficacy objective for this study was to evaluate the efficacy of induction treatment with R+Pola+Len, and consolidation treatment with R+Len in R/R DLBCL on the basis of the following endpoints:
CR at EOI, as determined by the investigator on the basis of PET-CT scans.
CR at EOI, as determined by the IRC and by the investigator on the basis of CT scans alone.
Objective response (defined as a CR or PR) at EOI, as determined by the IRC and by the investigator on the basis of PET-CT scans.
Objective response (defined as a CR or PR) at EOI, as determined by the IRC and by the investigator on the basis of CT scans alone.
Best response of CR or PR during the study, as determined by the investigator on the basis of CT scans alone.

Responses were determined on the basis of positron emission tomography (PET) and computed tomography (CT) scans or CT scans alone, using the Revised Lugano Response Criteria for Malignant Lymphoma (Cheson et al. 2014), hereinafter referred to as the Lugano 2014 criteria (see, Table 8). Responses were determined by an Independent Review Committee (IRC) and by the investigator.

C. Exploratory Efficacy Objectives

The exploratory efficacy objective for this study was to evaluate the long-term efficacy of R+Pola+Len on the basis of the following endpoints:
For patients who have positive PET scans at EOI:
   CR at end of consolidation (EOC), as determined by the IRC and by the investigator on the basis of PET-CT scans.
Progression-free survival (PFS), defined as the time from initiation of study treatment to first occurrence of disease progression or relapse, as determined by the investigator, or death from any cause.
Event-free survival (EFS), defined as the time from initiation of study treatment to any treatment failure, including disease progression or relapse, as determined by the investigator (e.g., on the basis of CT scans alone), initiation of new anti-lymphoma therapy, or death from any cause, whichever occurs first.
Disease-free survival (DFS), defined, among patients achieving a CR, as the time from the first occurrence of a documented CR to relapse, as determined by the investigator (e.g., on the basis of CT scans alone), or death from any cause, whichever occurs first.
Overall survival (OS), defined as the time from initiation of study treatment to death from any cause.

D. Pharmacokinetic Objective

The pharmacokinetic (PK) objective for this study was to characterize the PK profiles of rituximab, polatuzumab vedotin, and lenalidomide when given in combination, on the basis of the following endpoints:
Observed plasma/serum rituximab concentration.
Observed plasma/serum concentrations of polatuzumab vedotin and relevant analytes (total antibody (Tab), antibody-conjugated mono-methyl auristatin E (acMMAE), and unconjugated MMAE).
Observed plasma/serum lenalidomide concentration.

E. Immunogenicity Objectives

The immunogenicity objective for this study was to evaluate the immune response to rituximab and to polatuzumab vedotin on the basis of the following endpoints:
Incidence of human anti-chimeric antibodies (HACAs) to rituximab during the study relative to the prevalence of HACAs at baseline.
Incidence of anti-therapeutic antibodies (ATAs) to polatuzumab vedotin during the study relative to the prevalence of ATAs at baseline.

The exploratory immunogenicity objective for this study was to evaluate potential relationships between HACAs and ATAs on the basis of the following endpoint:
Correlation between HACA and ATA status and respective efficacy, safety, biomarker, or PK endpoints.

F. Biomarker Objectives

The exploratory biomarker objectives for this study were to identify non-inherited biomarkers that are predictive of response to study treatment (i.e., predictive biomarkers), are associated with progression to a more severe disease state (i.e., prognostic biomarkers), are associated with acquired resistance to study treatment, are associated with susceptibility to developing adverse events, can provide evidence of study treatment activity, can increase the knowledge and understanding of lymphoma biology or study treatment mechanism of action, or can contribute to improvement of diagnostic assays on the basis of the following endpoint:
Association between non-inherited biomarkers (see, Table 9) and efficacy, safety, pharmacokinetics, or immunogenicity endpoints.

### II. Study Design

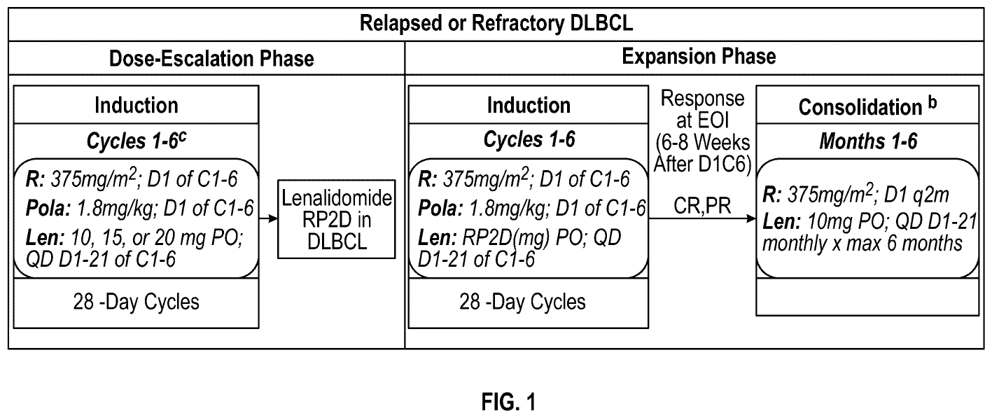

The study included an initial dose-escalation phase, followed by an expansion phase, during which polatuzumab vedotin and lenalidomide were given at their RP2Ds. An overview of the study is provided in FIG. 1.

A. Dose Escalation Phase

The purpose of the dose escalation phase was to identify the RP2D for lenalidomide when combined with polatuzumab vedotin at 1.8 mg/kg and rituximab at 375 mg/m² as induction treatment in patients with R/R DLBCL.

Patients enrolled in the DLBCL-dose-escalation phase received induction treatment, administered in 28-day cycles as shown in Table 1. When study treatments were given on the same day, they were administered sequentially in the following order: lenalidomide, rituximab, and polatuzumab vedotin.

TABLE 1
Induction Treatment for the Dose-Escalation Phase.

| Cycle | R+ Pola + Len (28-Day Cycles) |
|---|---|
| Cycles 1-6 | Lenalidomide 10 mg, 15 mg, or 20 mg PO once |
| | daily on Days 1-21. |
| | Rituximab 375 mg/m² IV on Day 1. |
| | Polatuzumab vedotin 1.8 mg/kg IV on Day 1. |

IV = intravenous; PO = by mouth.

Patients who achieved a CR or PR at EOI received consolidation treatment with R+Len, as described below, in Table 2. Polatuzumab vedotin was not given as consolidation treatment. Consolidation treatment started 8 weeks (±1 week) after Day 1 of Cycle 6 and continued for 6 months until disease progression or unacceptable toxicity.

TABLE 2
Consolidation Treatment for the Dose-Escalation Phase.

| Patient | |
|---|---|
| Population | Regimen |
| Patients with | Consolidation treatment consisting of the following, |
| DLBCL | administered for approximately 6 months |
| | (from Months 1-6): |
| | Lenalidomide 10 mg PO once daily on Days 1-21 of each |
| | month for a maximum of 6 months. |
| | Rituximab 375 mg/m² IV on Day 1 of every |
| | other month (i.e., every 2 months starting with Month 1 |
| | (i.e., Months 1, 3, 5). |

A month was defined as 28 days. Treatments were administered sequentially in the following order: lenalidomide followed by rituximab.

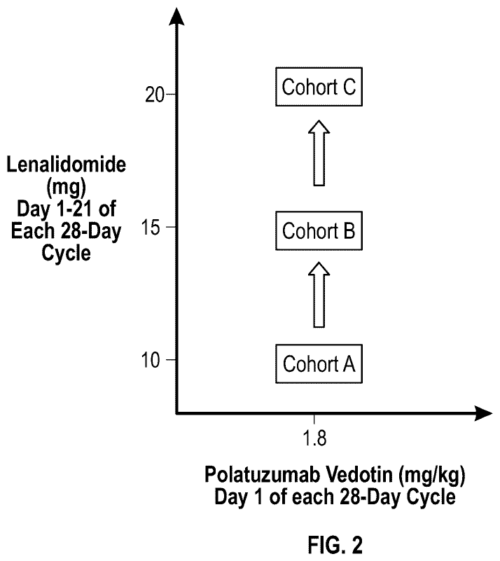

As shown in FIG. 2, a standard 3+3 dose-escalation schema was used. The rituximab and polatuzumab vedotin dose levels were fixed during the dose-escalation phase, and only the lenalidomide was dose escalated. Dose escalation cohorts A-C are described in Table 3.

TABLE 3
Dose-Escalation Cohorts.

| Cohort | Rituximab | Polatuzumab Vedotin | Lenalidomide |
|---|---|---|---|
| A | 375 mg/m² | 1.8 mg/kg | 10 mg |
| B | 375 mg/m² | 1.8 mg/kg | 15 mg |
| C | 375 mg/m² | 1.8 mg/kg | 20 mg |

Rituximab was administered at a dose of 375 mg/m² IV on Day 1 of each 28-day cycle of induction.
Polatuzumab vedotin was administered at a dose of 1.8 mg/kg IV on Day 1 of each 28-day cycle of induction.
Lenalidomide was administered at doses of 10 mg, 15 mg, or 20 mg PO once daily on Days 1-21 of each 28-day cycle.

If Cohort A doses were deemed safe and tolerable, escalation continued with enrollment of Cohort B. If Cohort B doses were deemed safe and tolerable, escalation continued with enrollment of Cohort C.

B. Expansion Phase

The expansion phase was designed to further assess the safety and efficacy of lenalidomide when combined with a fixed dose of rituximab and polatuzumab vedotin in DLBCL patients.

All patients enrolled in the expansion phase received induction treatment as outlined in Table 4. When study treatments were given on the same day, they were administered sequentially in the following order: lenalidomide, rituximab, and polatuzumab vedotin. During the expansion phase, patients received rituximab and polatuzumab in combination with lenalidomide at the RP2D.

TABLE 4
Induction Treatment for the Expansion Phase.

| Cycle | R+ Pola + Len (28-Day Cycles) |
|---|---|
| Cycles 1-6 | Lenalidomide 10 mg, 15 mg, or 20 mg PO once daily |
| | on Days 1-21. |
| | Rituximab 375 mg/m² IV on Day 1. |
| | Polatuzumab vedotin 1.8 mg/kg IV on Day 1. |

Patients who achieved a CR or PR at EOI received post-induction treatment (referred to as consolidation) with rituximab and lenalidomide as outlined in Table 5. Polatuzumab vedotin was not given during the post-induction phase. Post-induction treatment started 8 weeks (±1 week) after Day 1 of the final cycle of induction and continued until disease progression or unacceptable toxicity, for up to 6 months of consolidation treatment.

TABLE 5
Consolidation Treatment for the Expansion Phase.

| Patient | |
|---|---|
| Population | Regimen |
| Patients with | Consolidation treatment consisting of the following, |
| DLBCL | administered for approximately 6 months (Months 1-6): |
| | Lenalidomide 10 mg PO once daily on Days 1-21 of each |
| | month for a maximum of 6 months. |
| | Rituximab 375 mg/m² IV on Day 1 of every other month |
| | (i.e., every 2 months starting with Month 1 (i.e., Months |
| | 1, 3, 5). |

A month was defined as 28 days. Treatments were administered sequentially in the following order: lenalidomide followed by rituximab.

C. Dosing and Administration

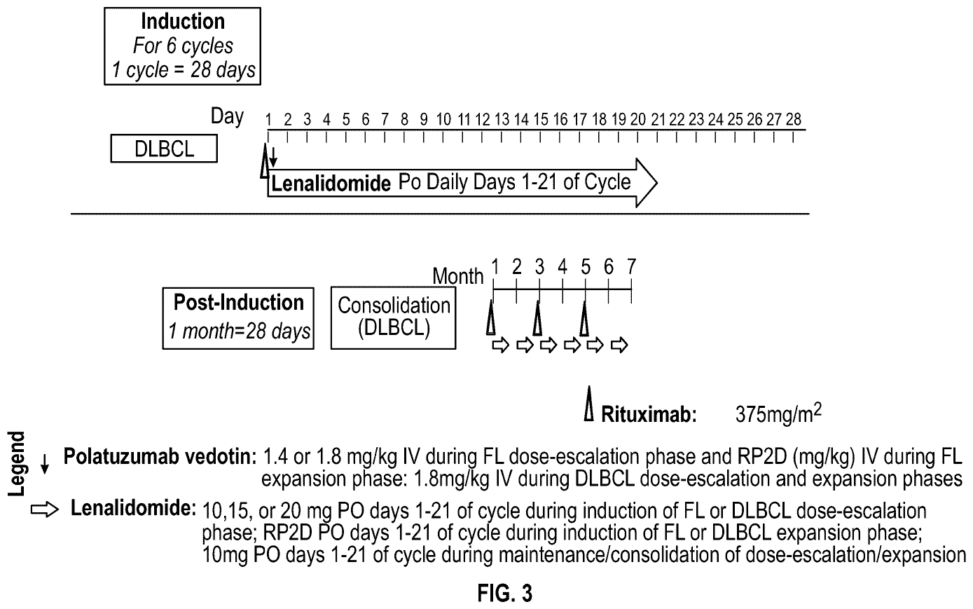

FIG. 3 provides an overview of the induction and post-induction treatment regimens used in this study.

Rituximab

Rituximab was administered by IV infusion at the dose of 375 mg/m² on Day 1 of Cycles 1-6 during induction treatment, and on Day 1 of every other month (i.e., every 2 months) during consolidation treatment.

The infusion of rituximab was split over 2 days if the patient was at increased risk for an infusion related reaction (high tumor burden or high peripheral lymphocyte count). Administration of rituximab was continued on the following day, if needed, for patients who experienced an adverse event during the rituximab infusion. If a dose of rituximab was split over 2 days, both infusions occurred with appropriate premedication and at the first infusion rate. Rituximab was administered as a slow IV infusion through a dedicated line.

Rituximab infusions were administered according to the instructions in Table 6.

TABLE 6
Administration of First and Subsequent Infusions of Rituximab.

| First Infusion (Day 1 of Cycle 1) | Subsequent Infusions |
|---|---|
| Begin infusion at an initial rate of 50 mg/hr. | If the patient experienced an infusion-related |
| If no infusion-related or hypersensitivity | or hypersensitivity reaction during the prior |
| reaction occurs, increase the infusion rate in 50- | infusion, use full premedication, including |
| mg/hr increments every 30 minutes to a | 100 mg of prednisone/prednisolone or 80 mg of |
| maximum of 400 mg/hr. | methylprednisolone or equivalent (until no |
| If a reaction develops, stop or slow the | further infusion related reaction occurs); begin |
| infusion. Administer medications and | infusion at an initial rate of 50 mg/hr; and |
| supportive care. If the reaction has resolved, | follow instructions for first infusion. |
| resume the infusion at a 50% reduction in rate | If the patient tolerated the prior infusion well |
| (i.e., 50% of rate being used at the time when | (defined by an absence of Grade 2 reactions |
| the reaction occurred). | during a final infusion rate of ≥100 mg/hr), |
| | begin infusion at a rate of 100 mg/hr. |
| | If no reaction occurs, increase the infusion |
| | rate in 100-mg/hr increments every |
| | 30 minutes to a maximum of 400 mg/hr. |
| | If a reaction develops, stop or slow the |
| | infusion. Administer medications and |
| | supportive care. If the reaction has resolved, |
| | resume the infusion at a 50% reduction in rate |
| | (i.e., 50% of rate being used at the time when |
| | the reaction occurred). |

Premedication with a corticosteroid, analgesic/antipyretic, and antihistamine was required to reduce the incidence and severity of infusion related reactions (IRRs).

Polatuzumab Vedotin

During the dose escalation phase and the expansion phase, the dose of polatuzumab vedotin was fixed at 1.8 mg/kg. Polatuzumab vedotin was administered by IV infusion on Day 1 of each cycle, during induction treatment only.

The initial dose was administered to well hydrated patients over 90 (±10) minutes. Premedication (e.g., 500-1000 mg of oral acetaminophen or paracetamol and 50-100 mg diphenhydramine) could be administered to an individual patient before administration of polatuzumab vedotin. Administration of corticosteroids was permitted at the discretion of the treating physician. If IRRs were observed with the first infusion in the absence of premedication, premedication was administered before subsequent doses.

The polatuzumab vedotin infusion was slowed or interrupted for patients experiencing infusion-associated symptoms. Following the initial dose, patients were observed for 90 minutes for fever, chills, rigors, hypotension, nausea, or other infusion-associated symptoms. If prior infusions were well tolerated, subsequent doses of polatuzumab vedotin were administered over 30 (±10) minutes, followed by a 30-minute observation period after the infusion.

Lenalidomide

Lenalidomide was administered orally once daily on Days 1-21 of Cycles 1-6 (28-day cycles) during induction treatment and on Days 1-21 of each month during consolidation. During the dose-escalation phase, lenalidomide was administered at a dose of 10, 15, or 20 mg. The dose was allowed to be deescalated to 5 mg. During the expansion phase, lenalidomide was administered at the RP2D during induction treatment and at 10 mg during consolidation treatment.

Lenalidomide increases the risk of thromboembolism (TE). All patients were required to take daily aspirin (75-100 mg) for TE prophylaxis during lenalidomide treatment and until 28 days after the last dose of lenalidomide. Patients who were unable to tolerate aspirin, patients with a history of TE, and patients at high risk of TE received warfarin or low-molecular-weight heparin (LMWH).

Table 7 provides an overview of the premedications administered in this study.

TABLE 7
Premedications.

| Timepoint | Patients Requiring Premedication | Premedication | Administration |
|---|---|---|---|
| Cycle 1, | All patients | Oral corticosteroid^{a} | Complete ≥1 hour |
| Day 1 | | | prior to rituximab |
| | | | infusion. |
| | All patients | Antihistamine drug^{b} | Administer ≥30 |
| | | Oral analgesic/ | minutes prior to |
| | | antipyretic^{c} | rituximab infusion. |
| | Patients at risk for TLS (e.g., | Allopurinol or suitable | Administer prior to |
| | because of bulky disease or | alternative, such as | rituximab infusion. |
| | renal impairment [creatinine | rasburicase, along with | |
| | clearance <70 mL/min]). | adequate hydration. | |
| Cycles 2 | Patients with no IRR during the | Oral analgesic/anti- | Premedication may be |
| and | previous infusion. | pyretic^{c} | omitted at the |
| Beyond, | | | investigator's |
| Day 1 | | | discretion. |
| | Patients with Grade 1 or 2 IRR | Antihistamine drug^{b} | Administer ≥30 |
| | during the previous infusion. | Oral analgesic/ | minutes prior to |
| | | antipyretic^{c} | rituximab infusion. |
| | Patients with Grade 3 IRR, | Oral corticosteroid^{a} | Complete ≥1 hour |
| | wheezing, urticarial, or other | | prior to rituximab |
| | symptoms of anaphylaxis during | | infusion. |
| | the previous infusion. | Antihistamine drug^{b} | Administer ≥30 |
| | Patients with bulky disease. | Oral analgesic/ | minutes prior to |
| | | antipyretic^{c} | rituximab infusion. |
| | Patients still at risk for TLS. | Allopurinol or suitable | Administer prior to |
| | | alternative, such as | rituximab infusion. |
| | | rasburicase, along with | |
| | | adequate hydration. | |

^{a}Treat with 100 mg of prednisone or prednisolone, 20 mg of dexamethasone, or 80 mg of methylprednisolone. Hydrocortisone should not be used, as it has not been effective in reducing rates of IRR.
^{b}For example, 50 mg of diphenhydramine.
^{c}For example, 1000 mg of acetaminophen/paracetamol.

Concomitant Therapies

TE prophylaxis treatment and premedication were administered as described above.

Hematopoietic growth factors were allowed. G-CSF was allowed to be administered in each cycle of therapy as primary prophylaxis for neutropenia, per American Society of Clinical Oncology (ASCO), EORTC, and European Society for Medical Oncology (ESMO) guidelines (Smith et al., J Clin Oncol (2006) 24:3187-205) or per institutional standards.

Erythropoietic agents or other agents that may increase the risk of thrombosis, such as estrogen-containing therapies (e.g., oral contraceptives), were used with caution, because of the increased risk of TE in patients taking lenalidomide.

Patients using concomitant medication that could possibly worsen thrombocytopenia-related events (e.g., platelet inhibitors and anticoagulants) could be at greater risk of bleeding. When possible, prior vitamin K antagonist therapy was replaced with LMWH prior to Day 1 of Cycle 1.

Patients who were receiving digoxin underwent periodic monitoring of digoxin plasma levels because of potential drug interactions with lenalidomide. A close monitoring of international normalized ratio (INR) and prothrombin time (PT) was recommended in patients receiving warfarin.

There is an increased risk of rhabdomyolysis when statins are administered with lenalidomide, which may be simply additive. Enhanced clinical and laboratory monitoring was undertaken when warranted, notably during the first weeks of treatment.

Patients who received strong CYP3A4 inhibitors or P-glycoprotein (P-gp) inhibitors in combination with polatuzumab vedotin were closely monitored for adverse reactions if any.

Prophylactic treatment with antibiotics was administered as per standard practice.

### III. Study Participants

Patients with DLBCL who met the eligibility criteria below were included in this study.

A. Inclusion and Exclusion Criteria

Patients who met the following inclusion criteria were included in this study:
Adults, aged 18 years or older.
Eastern Cooperative Oncology Group (ECOG) Performance Status of 0, 1, or 2.
Relapsed or refractory DLBCL (R/R DLBCL) after treatment with at least one prior chemoimmunotherapy regimen that included an anti-CD20 monoclonal antibody in patients who were not eligible for autologous stem-cell transplantation or who experienced disease progression following treatment with high-dose chemotherapy plus autologous stem-cell transplantation.
Histologically documented CD20-positive B-cell lymphoma.
Fluorodeoxyglucose (FDG)-avid lymphoma (i.e., PET-positive lymphoma).
At least one bi-dimensionally measurable lesion (>1.5 cm in its largest dimension by CT scan or magnetic resonance imaging [MRI]).

Patients who met any of the following exclusion criteria were excluded from this study:
History of transformation of indolent disease to DLBCL.
Known CD20-negative status at relapse or progression.
Central nervous system lymphoma or leptomeningeal infiltration.
Prior allogeneic stem cell transplantation (SCT).
Completion of autologous SCT within 100 days prior to Day 1 of Cycle 1.
History of resistance to lenalidomide or response duration of ≤1 year (for patients who had a response to a prior lenalidomide-containing regimen).
Prior standard or investigational anti-cancer therapy as specified below:
   Lenalidomide, fludarabine, or alemtuzumab within 12 months prior to Day 1 of Cycle 1; radioimmunoconjugate within 12 weeks prior to Day 1 of Cycle 1; monoclonal antibody or antibody-drug conjugate (ADC) therapy within 5 half-lives or 4 weeks prior to Day 1 of Cycle 1, whichever is longer; radiotherapy, chemotherapy, hormonal therapy, or targeted small-molecule therapy within 2 weeks prior to Day 1 of Cycle 1.
Clinically significant toxicity (other than alopecia) from prior therapy that has not resolved to Grade ≤2 (per NCI CTCAE, Version 4.0) prior to Day 1 of Cycle 1.
Treatment with systemic immunosuppressive medications, including, but not limited to, prednisone, azathioprine, methotrexate, thalidomide, and anti-tumor necrosis factor agents within 2 weeks prior to Day 1 of Cycle 1. Treatment with inhaled corticosteroids and mineralocorticoids was permitted. If corticosteroid treatment was urgently required for lymphoma symptom control prior to the start of study treatment, up to 100 mg/day of prednisone or equivalent were given for a maximum of 5 days, but all tumor assessments were completed prior to initiation of corticosteroid treatment.
History of severe allergic or anaphylactic reaction to humanized or murine monoclonal antibodies; known sensitivity or allergy to murine products or any component of rituximab, polatuzumab vedotin, or lenalidomide formulations.
History of erythema multiforme, Grade ≥3 rash, or desquamation (blistering) following prior treatment with immunomodulatory derivatives such as thalidomide and lenalidomide.
Active bacterial, viral, fungal, or other infection; positive for hepatitis B surface antigen (HBsAg), total hepatitis B core antibody (HBcAb), or hepatitis C virus (HCV) antibody at screening; known history of HIV positive status; vaccination with a live virus vaccine within 28 days prior to Day 1 of Cycle 1.
History of progressive multifocal leukoencephalopathy.
History of other malignancy that could affect compliance with the protocol or interpretation of results, with the exception of the following:
   Curatively treated carcinoma in situ of the cervix; good-prognosis ductal carcinoma in situ of the breast; basal- or squamous-cell skin cancer; Stage I melanoma; or low-grade, early-stage localized prostate cancer.
   Any previously treated malignancy that has been in remission without treatment for ≥2 years prior to enrollment.
Contraindication to treatment for thromboembolism (TE) prophylaxis.
Grade ≥2 neuropathy.
Evidence of any significant, uncontrolled concomitant disease that could affect compliance with the protocol or interpretation of results, including significant cardiovascular disease (such as New York Heart Association Class III or IV cardiac disease, myocardial infarction within the previous 6 months, unstable arrhythmia, or unstable angina) or significant pulmonary disease (such as obstructive pulmonary disease or history of bronchospasm).
Major surgical procedure other than for diagnosis within 28 days prior to Day 1 of Cycle 1 or anticipation of a major surgical procedure during the course of the study.
Inadequate renal, liver, or hematologic function (unless due to underlying lymphoma), defined as follows: hemoglobin ≤9 g/dL; absolute neutrophil count (ANC)≤1.5×10⁹/L; platelet count ≤75×10⁹/L.
Any of the following abnormal laboratory values (unless due to underlying lymphoma):
   Calculated creatinine clearance ≤50 mL/min (using the Cockcroft-Gault formula); aspartate aminotransferase (AST) or alanine transaminase (ALT)>2.5× upper limit of normal (ULN); serum total bilirubin >1.5×ULN (or >3×ULN for patients with Gilbert syndrome); international normalized ratio (INR) or prothrombin time (PT)>1.5×ULN in the absence of therapeutic anticoagulation; partial thromboplastin time (PTT) or activated partial thromboplastin time (aPTT)>1.5×ULN in the absence of a lupus anticoagulant.

IV. Study Assessments A. Clinical Parameters

The following clinical parameters relevant to disease history, diagnosis, and prognostic indices were recorded at screening:
ECOG Performance Status.
Ann Arbor staging.
International Prognostic Index
B symptoms (unexplained fever >38° C., night sweats, and unexplained weight loss >10% of body weight over 6 months)
Previous lines of anti-lymphoma treatment and response to prior therapy, date of disease progression in relation to start date of prior therapy, and date of last dose of prior therapy.

B. Tumor and Response Evaluations

Responses were assessed by the IRC and the investigator on the basis of PET and CT scans, using the Lugano 2014 criteria, taking into account results of bone marrow examinations for patients with bone marrow involvement at screening.

In this study, the Lugano 2014 criteria for a PET-CT-based CR were slightly modified to require normal bone marrow for patients with bone marrow involvement at screening. If indeterminate by morphology, immunohistochemistry should be negative. Additionally, designation of PET-CT-based PR required that CT-based response criteria for a CR or PR be met in addition to the PET-CT-based response criteria for a PR.

Table 8 provides a summary of the Modified Lugano criteria.

TABLE 8
Modified Lugano Response Criteria for Malignant Lymphoma (Cheson et al. 2014).

| Response and Site | PET-CT-Based Response | CT-Based Response |
|---|---|---|
| Complete | Complete metabolic response | Complete radiologic response |
| | | (all of the following). |
| Lymph nodes and | Score 1, 2, or 3^{a} with or | Target nodes/nodal masses |
| extralymphatic sites | without a residual mass on | must regress to ≤1.5 cm in |
| | 5PS^{b}. | LDi. |
| | It is recognized that in | No extralymphatic sites of |
| | Waldeyer's ring or extranodal | disease. |
| | sites with high physiologic | |
| | uptake or with activation | |
| | within spleen or marrow (e.g., | |
| | with chemotherapy or myeloid | |
| | colony-stimulating factors), | |
| | uptake may be greater than | |
| | normal mediastinum and/or | |
| | liver. In this circumstance, | |
| | complete metabolic response | |
| | may be inferred if uptake at | |
| | sites of initial involvement is | |
| | no greater than surrounding | |
| | normal tissue even if the tissue | |
| | has high physiologic uptake. | |
| Non-measured lesion | Not applicable | Absent |
| Organ enlargement | Not applicable | Regress to normal |
| New lesions | None | None |
| Bone marrow | No evidence of FDG-avid | Normal by morphology; if |
| | disease in marrow | indeterminate, IHC negative. |
| Partial | Partial metabolic response | Partial remission (all of the |
| | | following). |
| Lymph nodes and | Score 4 or 5^{b} with reduced | ≥50% decrease in SPD of up |
| extralymphatic sites | uptake compared with baseline | to 6 target measurable nodes |
| | and residual mass(es) of any | and extranodal sites. |
| | size. | When a lesion is too small to |
| | At interim, these findings | measure on CT, assign |
| | suggest responding disease. | 5 mm × 5 mm as the default |
| | At end of treatment, these | value. |
| | findings indicate residual | When no longer visible, 0 × 0 |
| | disease. | mm. |
| | | For a node >5 mm × 5 mm but |
| | | smaller than normal, use actual |
| | | measurement for calculation. |
| Non-measured lesion | Not applicable | Absent/normal, regressed, but |
| | | no increase. |
| Organ enlargement | Not applicable | Spleen must have regressed |
| | | by >50% in length beyond |
| | | normal. |
| New lesions | None | None |
| Bone marrow | Residual uptake higher than | Not applicable |
| | uptake in normal marrow but | |
| | reduced compared with | |
| | baseline (diffuse uptake | |
| | compatible with reactive | |
| | changes from chemotherapy | |
| | allowed). If there are persistent | |
| | focal changes in the marrow in | |
| | the context of a nodal response, | |
| | consideration should be given | |
| | to further evaluation with MRI | |
| | or biopsy or an interval scan. | |
| No response or stable disease | No metabolic response | Stable disease |
| Target nodes/nodal masses, | Score 4 or 5^{b} with no | <50% decrease from baseline |
| extranodal lesions | significant change in FDG | in SPD of up to 6 dominant, |
| | uptake from baseline at interim | measurable nodes and |
| | or end of treatment | extranodal sites; no criteria for |
| | | progressive disease are met. |
| Non-measured lesion | Not applicable | No increase consistent with |
| | | progression. |
| Organ enlargement | Not applicable | No increase consistent with |
| | | progression. |
| New lesions | None | None |
| Bone marrow | No change from baseline | Not applicable |
| Progressive disease | Progressive metabolic disease | Progressive disease requires at |
| | | least 1 of the following: |
| Individual target nodes/nodal | Score 4 or 5^{b} with an increase | PPD progression |
| masses | in intensity of uptake from | |
| | baseline; and/or | |
| Extranodal lesions | New FDG-avid foci consistent | An individual node/lesion must |
| | with lymphoma at interim or | be abnormal with: |
| | end-of-treatment assessment. | LDi >1.5 cm and increase by ≥50% |
| | | from PPD nadir and an increase |
| | | in LDi or SDi from nadir |
| | | 0.5 cm for lesions ≤2 cm |
| | | 1.0 cm for lesions >2 cm |
| | | In the setting of splenomegaly, |
| | | the splenic length must |
| | | increase by >50% of the extent |
| | | of its prior increase beyond |
| | | baseline (e.g., a 15-cm spleen |
| | | must increase to >16 cm). If |
| | | no prior splenomegaly, must |
| | | increase by at least 2 cm from |
| | | baseline. |
| | | New or recurrent splenomegaly |
| | | New or clear progression of |
| | | preexisting non-measured |
| | | lesions. |
| New lesions | New FDG-avid foci consistent | Regrowth of previously |
| | with lymphoma rather than | resolved lesions. |
| | another etiology (e.g., | A new node >1.5 cm in any |
| | infection, inflammation); if | axis. |
| | uncertain regarding etiology of | A new extranodal site >1.0 cm |
| | new lesions, biopsy or interval | in any axis; if <1.0 cm in |
| | scan may be considered. | any axis, its presence must be |
| | | unequivocal and must be |
| | | attributable to lymphoma. |
| | | Assessable disease of any size |
| | | unequivocally attributable to |
| | | lymphoma. |
| Bone marrow | New or recurrent FDG-avid | New or recurrent involvement. |
| | foci. | |

5PS = 5-point scale; FDG = fluorodeoxyglucose; LDi = longest transverse diameter of a lesion; PPD = cross product of the LDi and perpendicular diameter; SDi = shortest axis perpendicular to the LDi; SPD = sum of the product of the perpendicular diameters for multiple lesions.
^{a}A score of 3 in many patients indicates a good prognosis with standard treatment, especially if at the time of an interim scan. However, in trials involving PET where de-escalation is investigated, it may be preferable to consider a score of 3 as inadequate response (to avoid under-treatment). Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (e.g., liver, spleen, kidneys, lungs), GI involvement, cutaneous lesions, or those noted on palpation. Non-measured lesions: Any disease not selected as measured; dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
^{b}PET 5PS: 1 = no uptake above background; 2 = uptake ≤ mediastinum; 3 = uptake > mediastinum but ≤ liver; 4 = uptake moderately > liver; 5 = uptake markedly higher than liver and/or new lesions; X = new areas of uptake unlikely to be related to lymphoma.

C. Radiographic Assessments

PET scans included the base of the skull to mid-thigh region. Full body PET scans were performed when clinically appropriate. CT scans with oral and IV contrast included chest, abdomen, and pelvic scans. CT scans of the neck were included if clinically indicated (i.e., if evidence of disease upon physical examination) and were repeated throughout the study if there was disease involvement at baseline. If contrast was medically contraindicated (e.g., patients with contrast allergy or impaired renal clearance), MRI scans of the chest, abdomen, and pelvis (and neck, if clinically indicated) and a non-contrast CT scan of the chest was performed. If MRI scans could not be obtained, CT scans without contrast were permitted as long as they allowed consistent and precise measurement of the targeted lesions during the study treatment period. The same radiographic assessment modality was used for all response evaluations. A full tumor assessment, including radiographic assessment, was performed any time disease progression or relapse was suspected.

D. Bone Marrow Assessments

Bone marrow examinations were required at screening for staging purposes in all patients and were performed within approximately 3 months prior to Day 1 of Cycle 1. If bone marrow infiltration was present at screening, a bone marrow biopsy was required at the EOI response assessment for all patients who may have achieved a CR. In patients with a PR and continued bone marrow involvement, a subsequent bone marrow examination was used to confirm a CR at a later time point. E. Laboratory Assessments

Samples for the following laboratory tests were analyzed:
Hematology: hemoglobin, hematocrit, platelet count, red blood cell (RBC) count, white blood cell (WBC) count, and percent or absolute WBC differential count (neutrophils, eosinophils, basophils, monocytes, lymphocytes, other cells).
Chemistry panel (serum or plasma): sodium, potassium, glucose, BUN or urea, creatinine, calculated creatinine clearance, calcium, total bilirubin, direct bilirubin, total protein, albumin, ALT, AST, alkaline phosphatase, LDH, uric acid, glycosylated hemoglobin (HbA1c), amylase, and lipase (amylase and lipase only during induction).
Thyroid-stimulating hormone, triiodothyronine, thyroxine
β2 microglobulin.
Coagulation: INR, aPTT (or PTT), and PT.
Viral serology: Hepatitis B testing included HBsAg and total HBcAb; Hepatitis C testing included HCV antibody; HIV testing.
Quantitative immunogloblulins: IgA, IgG, and IgM.
Serum samples for rituximab PK analysis using a validated assay.
Serum and plasma samples for polatuzumab vedotin PK analysis using a validated assay.
Plasma samples for lenalidomide PK analysis using a validated assay.
Serum samples for assessment of rituximab HACAs using a validated assay.
Serum samples for assessment of polatuzumab vedotin ATAs using a validated assay.
Tumor tissue samples (obtained within 6 months prior to the initiation of study treatment for DLBCL) and the corresponding pathology report for retrospective central confirmation of the diagnosis of DLBCL and for assessment of candidate biomarkers.
Tumor biopsy samples obtained at the time of progression for assessment of candidate biomarkers.
Plasma and whole blood samples for assessment of candidate biomarkers.
Whole blood for lymphocyte immunophenotyping.

F. Biomarker Assessments

Biomarkers assessed in this study included DLBCL cell-of-origin prognostic subgroups (ABC and GCB), Bcl-2 overexpression, Myc-positivity, BCL2 rearrangements, overexpression of Bcl-2 and Myc, CD79b expression, and Minimal Residual Disease (MR). In addition, biomarkers associated with disease biology (immune gene expression profiles and disease subtype gene expression patterns and associated mutations, i.e., MYD88 and CD79b), mechanism of action of study drugs (i.e., including but not limited to regulated substrates of lenalidomide, i.e., CRBN, MYC, IRF4, or immune repertoire signatures), and mechanisms of resistance were assessed.

A summary of biomarkers included in this study is provided in Table 9.

TABLE 9
Non-Inherited Biomarkers.

| Sample Type | Timing | Non-Inherited Biomarkers |
|---|---|---|
| Archival or fresh pretreatment | Prior to study (archival) or | DLBCL cell-of-origin subtype |
| and progression tumor tissue. | baseline (fresh) and at | (ABC vs. GCB), BCL2, MYC. |
| | disease progression | Target expression BCL2 and |
| | | CD79b, immune infiltrate, |
| | | cereblon (and surrogates). |
| | | Lymphoma-related genetic |
| | | changes (DNA) and gene |
| | | expression (mRNA) or protein |
| | | expression (IHC associated |
| | | with response or potential |
| | | resistance). |
| | | Lymphoma index clone in |
| | | minimal residual disease |
| | | (MRD). |
| Plasma isolated from whole | Baseline and subsequent | Circulating lymphoma cells |
| blood. | timepoints during treatment | and/or cell-free circulating |
| | (patients in the expansion | tumor DNA (detection of |
| | phase only). | minimal residual disease). |
| Whole blood | Baseline and subsequent | Lymphocyte |
| | timepoints during and after | immunophenotyping, including |
| | treatment. | B-cell counts (CD19), T-cell |
| | | counts (CD3, CD4, and CD8), |
| | | and NK-cell counts (CD16 and |
| | | CD56). |
| Plasma | Baseline (pre-dose and | Cytokines characteristic of |
| | post-dose) and subsequent | T-cell activation and |
| | timepoints (pre-dose) during | lenalidomide activity (e.g., |
| | treatment. | IL-8 and IFNγ). |

ABC = activated B cell-like; GCB = germinal-center B cell-like; IHC = immunohistochemistry; NK-cell = natural killer cell.

G. Safety Assessments

Adverse events were assessed based on the National Cancer Institute Common Terminology Criteria for Adverse Events (NCI CTCAE; Version 4.0). Adverse events that are not specifically listed in the NCI CTCAE were graded as follows:
Grade 1: Mild; asymptomatic or mild symptoms; clinical or diagnostic observations only; or intervention not indicated.
Grade 2: Moderate; minimal, local, or non-invasive intervention indicated; or limiting age-appropriate instrumental activities of daily living.
Grade 3: Severe or medically significant, but not immediately life threatening; hospitalization or prolongation of hospitalization indicated; disabling; or limiting self-care activities of daily living.
Grade 4: Life-threatening consequences or urgent intervention indicated.
Grade 5: Death related to adverse event.

H. Study Populations

The following populations were defined:
The primary safety and efficacy populations included patients who received at least one dose of any component of the treatment combination.
The intent-to-treat population included patients enrolled in the study.

V. Adverse Events, Dose Modifications/Reductions, and Management of Toxicities

### A. Specific Adverse Events and Dose Modifications/Reductions

Polatuzumab Vedotin

Neutropenia and peripheral neuropathy are identified risks of polatuzumab vedotin. Potential risks of polatuzumab vedotin include infections, PML, infusion-related reactions, tumor lysis syndrome, bone marrow toxicity, immunogenicity, reproductive toxicity, gastrointestinal toxicity, hyperglycemia, and hepatotoxicity.

The dose of polatuzumab vedotin was reduced due to neurotoxicity only according to the dose reduction steps outlined in Table 10, based on the starting dose.

TABLE 10
Polatuzumab Vedotin Dose-Reduction Steps.

Dose Reduction

| Starting Dose | Step 1 | Step 2 |
|---|---|---|
| 1.8 mg/kg | 1.4 mg/kg | none |
| 1.4 mg/kg | none | none |

Lenalidomide

Risks associated with lenalidomide include embryo-fetal toxicity, neutropenia, thrombocytopenia, venous and arterial thromboembolism, tumor flare reaction (TFR), severe skin reactions, tumor lysis syndrome, hepatotoxicity, renal impairment, thyroid disorders, peripheral neuropathy, second primary malignancies, cardiovascular reactions, cardiac toxicities, and impaired stem cell mobilization.

The dose of lenalidomide was reduced in 5-mg increments one or two times during induction or post-induction, depending on the starting dose, as outlined in Table 11. No more than one dose reduction per treatment cycle occurred. If the lenalidomide dose was reduced to 5 mg during induction, the consolidation dose was allowed to be escalated to start at 10 mg in post-induction. In all other cases, if the lenalidomide dose was reduced, re-escalation was not permitted.

TABLE 11
Lenalidomide Dose-Reduction Steps.

Dose Reduction

| Starting Dose | Step 1 | Step 2 |
|---|---|---|
| 20 mg | 15 mg | 10 mg |
| 15 mg | 10 mg | 5 mg |
| 10 mg | 5 mg | none |

If a lenalidomide-related toxicity occurred during lenalidomide treatment (i.e., before Day 21 of the cycle), lenalidomide was withheld until criteria for recovery were met (i.e., improved to Grade ≤2 or baseline values). If recovery was observed prior or on Day 15 of the cycle, lenalidomide was resumed at the same dose for the remainder of the cycle (through Day 21; missed doses were not made up). If resuming lenalidomide at the same dose within the cycle represented an unacceptable risk for the patient, lenalidomide was resumed at reduced dose or withheld for the remainder of the cycle. For subsequent cycles, lenalidomide was resumed at reduced doses. If recovery was observed after Day 15 of the cycle, lenalidomide was not resumed for the current cycle. For subsequent cycles, lenalidomide was resumed at reduced doses.

Rituximab

The following adverse events are considered to be important risks associated or potentially associated with rituximab: IRRs, infections (including severe infections), progressive multifocal leukoencephalopathy (PML), hepatitis B reactivation, neutropenia (including prolonged neutropenia), tumor lysis syndrome (TLS), impaired immunization response, severe skin reactions (Stevens-Johnson syndrome [SJS]/toxic epidermal necrolysis [TEN]), and gastrointestinal (GI) perforation.

There were no dose reductions of rituximab.

Tumor Lysis Syndrome Prophylaxis

Patients who were considered to have a high tumor burden (e.g., lymphocyte count ≥25×10⁹/L or bulky lymphadenopathy) and who were considered to be at risk for tumor lysis received tumor lysis prophylaxis (e.g., allopurinol ≥300 mg/day orally or a suitable alternative treatment starting 12-24 hours before study treatment) and were well hydrated before the initiation of study treatment on Day 1 of Cycle 1. Patients continued to receive repeated prophylaxis with allopurinol and adequate hydration before each subsequent infusion.

B. Management of Toxicities during Induction Treatment Management of Hematologic Toxicities during Induction

Hematologic toxicity was defined as neutropenia, anemia, or thrombocytopenia. Lymphopenia was not considered a hematologic toxicity, but rather an expected outcome of therapy. Table 12 provides guidelines for management of hematologic toxicities that occurred during induction treatment.

TABLE 12
Guidelines for Management of Hematologic Toxicities
that Occurred during Induction Treatment.

| Event | Action to Be Taken |
|---|---|
| Grade 3 or 4 | For patients on a lenalidomide dose ≥10 mg who have had one or no prior |
| hematologic | lenalidomide dose reductions: |
| toxicity ^{a, b} | Withhold study treatment.^{a} |
| | Administer RBCs or platelets as required. |
| | If patient has not already initiated G-CSF, initiate prophylactic G-CSF for |
| | current and subsequent cycles. |
| | For patients who develop platelet count of <20,000/μL while receiving |
| | LMWH, reduce the dose of LMWH. For patients who develop platelet count |
| | of <20,000/μL while receiving platelet inhibitors, consider temporarily |
| | withholding platelet inhibitors. |
| | Permanently discontinue study treatment if any of the following events occur: |
| | Grade 3 or 4 thrombocytopenia that results in significant bleeding. |
| | Recurrent Grade 3 or 4 neutropenia associated with fever >38° C. lasting >5 |
| | days or documented infection despite use of G-CSF and after one lenalidomide |
| | dose reduction. |
| | Recurrent Grade 4 neutropenia or thrombocytopenia lasting >7 days despite |
| | use of G-CSF (for neutropenia) and after one lenalidomide dose reduction. |
| | If improvement to Grade ≤2 or baseline ≤14 days after the scheduled date for |
| | the next cycle, resume rituximab and polatuzumab vedotin at full dose and |
| | resume lenalidomide at current dose. |
| | If improvement to Grade ≤2 or baseline 15-21 days after the scheduled date |
| | for the next cycle, resume rituximab and polatuzumab vedotin at full dose and |
| | resume lenalidomide at a reduced dose^{a, b} for current and subsequent cycles. |
| | If study treatment is withheld for >21 days, permanently discontinue study |
| | treatment. |
| | For patients who have had two prior dose reductions: |
| | Permanently discontinue study treatment. |

G-CSF = granulocyte colony-stimulating factor; LMWH = low-molecular-weight heparin.
^{a}Treatment delays apply to all toxicities; dose modifications apply only to toxicities that are considered to be related to any of the study treatment components. Toxicities that occur during the cycle and subside prior to the next cycle should not trigger the suggested dose modifications.
^{b}If cytopenia is thought to be caused mainly by B-cell lymphoma infiltration of the bone marrow, the lenalidomide dose may not be reduced.

Non-Hematologic Toxicities during Induction

Table 13 provides guidelines for management of non-hematologic toxicities that occur during induction treatment.

TABLE 13
Guidelines for Management of Non-Hematologic Toxicities during Induction.

| Event | Action to be Taken |
|---|---|
| General guidance for treatment | If study treatment is withheld for >21 days because of a |
| delays and discontinuation | toxicity that is attributable to study treatment, permanently |
| | discontinue study treatment. |
| | When a treatment cycle is delayed because of toxicity resulting |
| | from any component of the regimen, all study treatment should |
| | be held and resumed together to remain synchronized. |
| | If one drug is discontinued, treatment with the other two drugs |
| | may be continued for patients experiencing clinical benefit. |
| IRRs and anaphylaxis | IRRs are managed as described herein. |
| | In case of anaphylaxis, study treatment should be permanently |
| | discontinued. |
| Renal toxicity | Adjust the dose of lenalidomide^{a} as outlined below: |
| | If creatinine clearance is ≥30 but <50 mL/min, lenalidomide |
| | should be given at a dose of 10 mg/day. |
| | If creatinine clearance is <30 mL/min and dialysis is not |
| | required, lenalidomide should be given at a dose of 10 mg every |
| | other day. |
| | If creatinine clearance is <30 mL/min and dialysis is required, |
| | lenalidomide should be given at a dose of 5 mg/day. On dialysis |
| | days, the dose should be administered after dialysis. |
| Clinical tumor lysis syndrome | Withhold study treatment. |
| (TLS)^{b} | Correct electrolyte abnormalities, monitor renal function and |
| | fluid balance, and administer supportive care, including dialysis |
| | as indicated. Rasburicase therapy may be administered as needed |
| | to reduce hyperuricemia. |
| | If symptoms resolve completely, resume rituximab and |
| | polatuzumab vedotin at full dose and resume lenalidomide at a |
| | reduced dose as described herein for current and subsequent |
| | cycles. |
| | Perform chemistry panel every other day for the first week |
| | after re-initiation of lenalidomide. |
| Laboratory TLS^{b} | Withhold study treatment. |
| | Correct electrolyte abnormalities, monitor renal function and |
| | fluid balance, and administer supportive care as clinically |
| | indicated. |
| | If laboratory abnormalities have resolved completely, resume |
| | rituximab and polatuzumab vedotin at full dose and resume |
| | lenalidomide at a reduced dose as described herein for current |
| | and subsequent cycles. |
| New-onset neurologic | Withhold study treatment.^{a} |
| manifestations | If PML is ruled out, resume rituximab at full dose and resume |
| suggestive of PML | polatuzumab vedotin and lenalidomide at current dose. |
| | If PML is confirmed, permanently discontinue study treatment. |
| AST, ALT, or bilirubin | Withhold study treatment and monitor liver enzymes at least |
| increase: | every 7 days. |
| Grade ≥3 (or ≥10× ULN for | If improvement to Grade ≤1, resume rituximab and |
| patients with liver involvement) | polatuzumab vedotin at full dose and resume lenalidomide at a |
| | reduced dose as described herein for current and subsequent |
| | cycles. |
| | Permanently discontinue study treatment for life-threatening |
| | liver toxicity. |
| Tumor flare reaction, | Withhold study treatment. |
| Grade 3-4^{c} | Administer corticosteroids, NSAIDs, and/or narcotic |
| | analgesics. |
| | If improvement to Grade ≤1, resume rituximab and |
| | polatuzumab vedotin at full dose and resume lenalidomide at a |
| | reduced dose as described herein for current and subsequent |
| | cycles. |
| Tumor flare reaction, | Continue study treatment. |
| Grade 1-2^{c} | Administer corticosteroids, NSAIDs, and/or narcotic |
| | analgesics. |
| Neurotoxicity, Grade 4 | Permanently discontinue polatuzumab vedotin and all other |
| | study treatments. |
| Neurotoxicity, Grade 2 or 3 | Withhold study treatment.^{a} |
| | If improvement to Grade ≤1 within 21 days, resume study |
| | treatments for current and subsequent cycles as follows: |
| | Resume rituximab at full dose. |
| | For patients who started at 1.8 mg/kg, resume polatuzumab |
| | vedotin at a reduced dose of 1.4 mg/kg as described herein; for |
| | patients who started at 1.4 mg/kg, permanently |
| | discontinue polatuzumab vedotin.^{a} |
| | Resume lenalidomide at a reduced dose as described herein. |
| Dermatologic toxicity, | Permanently discontinue study treatment. |
| Grade 3 with blistering or | |
| Grade 4 | |
| Dermatologic toxicity, | First occurrence: |
| Grade 2 or Grade 3 without | Withhold study treatment and evaluate patient at least every 7 |
| blistering | days. |
| | Topical or parenteral corticosteroids may be required. |
| | If improvement to Grade ≤1, resume rituximab and |
| | polatuzumab vedotin at full dose and consider resuming |
| | lenalidomide at a reduced dose^{a} as described herein or continue |
| | current dose for current and subsequent cycles. |
| | Permanently discontinue all drugs in the event of angioedema, |
| | exfoliative or bullous rash, or if SJS or TEN is suspected. |
| | Second occurrence: |
| | Permanently discontinue study treatment. |
| Venous thrombosis or | Withhold lenalidomide. |
| embolism | Start anticoagulation treatment. |
| | After patient has been stabilized on anticoagulants and any |
| | complications of the thromboembolic event have been managed, |
| | lenalidomide may be resumed at current dose, dependent upon a |
| | benefit-risk assessment. |
| | Anticoagulants should be continued during the course of |
| | lenalidomide treatment. |
| Other Grade 3 or 4 non- | Grade 4 events: |
| hematologic toxicities (i.e., not | Permanently discontinue study treatment. |
| described above), excluding | Grade 3 events: |
| alopecia, nausea, and vomiting | Withhold study treatment. |
| | If improvement to Grade ≤1 or baseline, resume rituximab at |
| | full dose and, if the event is considered related to lenalidomide, |
| | resume lenalidomide accordingly at a reduced dose as described |
| | herein for current and subsequent cycles. |
| | No more than two dose reductions of lenalidomide are |
| | allowed. |
| Other Grade 2 non-hematologic | Withhold study treatment. |
| toxicities (i.e., not described | If improvement to Grade ≤1 or baseline, resume rituximab and |
| above), excluding alopecia, | polatuzumab vedotin at full dose and consider resuming |
| nausea, and vomiting | lenalidomide at a reduced dose^{a} as described herein or continue |
| | current dose for current and subsequent cycles. |

^{a}Dose modifications apply only to events that are considered to be related to lenalidomide.
^{b}According to Cairo-Bishop classification system.
^{c}Graded according to NCI CTCAE, Version 3.0.

C. Management of Toxicities during Consolidation Treatment

Table 14 provides guidelines for management of toxicities that occur during consolidation treatment.

TABLE 14
Guidelines for Management of Toxicities that Occur during Consolidation Treatment.

| Event | Action to be Taken |
|---|---|
| Hematologic toxicity: | Withhold rituximab and lenalidomide. |
| Grade 3 or 4 | Administer G-CSF for neutropenia. |
| | Administer RBCs or platelets as required. |
| | If improvement to Grade ≤2, resume rituximab and |
| | lenalidomide at same dose. Lenalidomide dose may be reduced |
| | by one dose level. |
| | If study treatment is withheld for >42 days, permanently |
| | discontinue study treatment. |
| Non-hematologic toxicity: | Withhold rituximab and lenalidomide. |
| Grade ≥2 | If improvement to Grade ≤1 or baseline, administer study |
| | treatment at full dose. Lenalidomide dose may be reduced by |
| | one dose level. |
| | If study treatment is withheld for >42 days, permanently |
| | discontinue study treatment. |

### Example 2: A Primary Analysis of a Phase Ib/II Study of an Anti-CD79b Immunoconjugate in Combination with an Anti-CD20 Antibody and an Immunomodulatory Agent in Relapsed or Refractory Diffuse Large B-Cell Lymphoma (DLBCL)

This Example describes a primary analysis of the Phase Ib/II study described in Example 1, evaluating the safety and efficacy of an anti-CD20 antibody (rituximab; also referred to herein as “R”) in combination with an anti-CD79b immunoconjugate (polatuzumab vedotin; also referred to herein as “Pola”) and an immunomodulatory agent (lenalidomide; also referred to herein as “Len”) in patients with relapsed or refractory diffuse large B-cell lymphoma (R/R DLBCL).

### I. METHODS

As described in detail in Example 1, patients with R/R DLBCL received induction treatment with six 28-day cycles of treatment with polatuzumab vedotin, rituximab, and lenalidomide (Pola-R-Len) as follows:
Polatuzumab vedotin was administered intravenously at a dose of 1.8 mg/kg on day 1 of cycles 1-6.
Rituximab was administered intravenously at a dose of 375 mg/m² on day 1 of cycles 1-6.
Lenalidomide was administered orally at doses between 10 mg and 20 mg (during a dose escalation phase), or at the recommended Phase II dose (RP2D), daily on days 1-21.

Patients who achieved a response at the end of induction (EOI) received 6 months of consolidation treatment with rituximab administered intravenously at a dose of 375 mg/m² on day 1 of every 2 months, and lenalidomide administered orally at a dose of 10 mg on days 1-21 monthly.

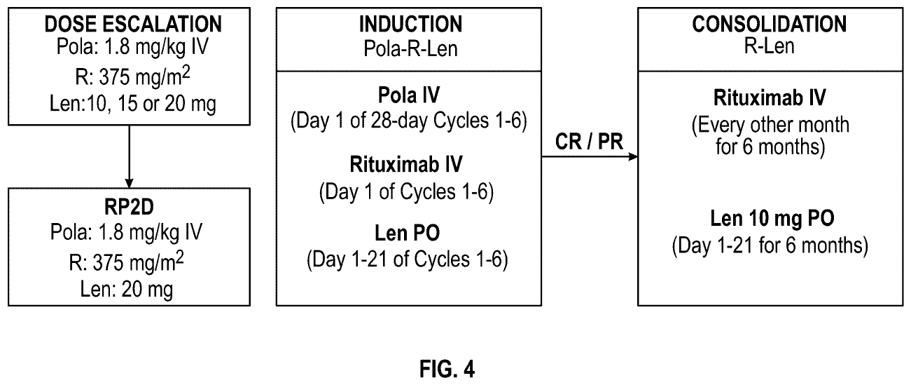

An overview of the study design is provided in FIG. 4.

The primary endpoints of this study were safety and tolerability, as well as positron emission tomography (PET)-complete response (CR) rate at EOI, assessed by an independent review committee (IRC) using the modified Lugano criteria.

Results of a primary analysis of this Phase Ib/II study are described below.

### II. RESULTS

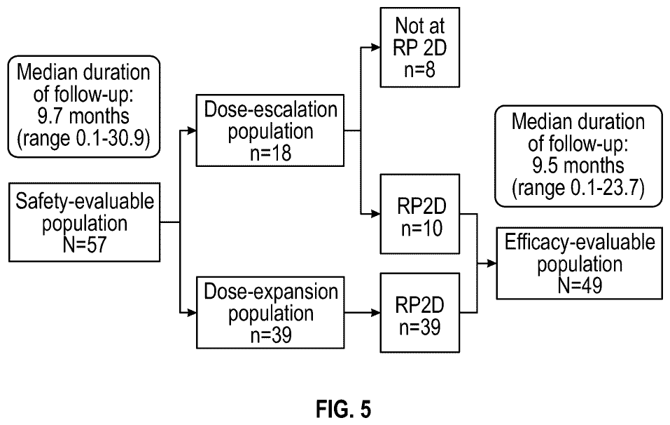

At primary analysis, 57 patients were enrolled in the study. FIG. 5 provides an overview of the primary analysis study populations.

A. Patient Characteristics

A summary of patient characteristics is provided in Table 15.

TABLE 15
Patient Characteristics.

| | Safety Evaluable | | Efficacy Evaluable | |
|---|---|---|---|---|
| Characteristics | (N = 57) | | (N = 49) | |
| Age in years, median (range) | 71 | (28-92) | 72 | (28-92) |
| Male sex, n (%) | 38 | (67) | 31 | (63) |
| Baseline ECOG PS 2 | 4 | (7) | 4 | (8) |
| Ann Arbor Stage III/IV, n (%) | 49 | (86) | 41 | (84) |
| IPI ≥3 at enrollment, n (%) | 34 | (60) | 31 | (63) |
| Number of prior therapies, | 2 | (1-8) | 2 | (1-7) |
| median (range) | | | | |
| 1, n (%) | 22 | (39) | 21 | (43) |
| 2, n (%) | 16 | (28) | 13 | (27) |
| ≥3, n (%) | 19 | (33) | 15 | (31) |
| Prior CAR-T therapy, n (%) | 3 | (5) | 3 | (6) |
| Bulky disease (≥7 cm), n (%) | 30 | (53) | 26 | (53) |
| Prior bone marrow transplant, n | 6 | (11) | 6 | (12) |
| (%) | | | | |
| Refractory, n (%) | | | | |
| Last prior therapy* | 37 | (65) | 31 | (63) |
| Primary refractory^{†} | 28 | (52) ^{‡} | 24 | (51) ^{¶} |

ECOG PS, Eastern Cooperative Oncology Group performance status; IPI, international prognostic index; CAR-T, chimeric antigen receptor T cell therapy.
*Defined as no response or progression or relapse within 6 months of last anti-lymphoma therapy end date.
^{†}Defined as no response or progression or relapse within 6 months of first anti-lymphoma therapy end date.
^{‡} Value shown is 28 of 54 patients (refractory status was unknown in three patients);
^{¶} Value shown is 24 of 47 patients (refractory status was unknown in two patients).

B. Safety

Overall, 56 patients (98%) experienced >1 adverse event (AE) during the study. Febrile neutropenia was reported in one patient (2%). Peripheral neuropathy occurred in 10 patients (18%); all cases were Grade 1 or 2. A summary of AEs occurring in >15% of patients is provided in Table 16.

TABLE 16
AEs Occurring in ≥15% of Patients.

| | | Safety-evaluable (N = 57) | | |
|---|---|---|---|---|
| AE, n (%) | | All grade | Grade 3-4 | |
| Hematologic AEs | Neutropenia | 35 (61) | 33 | (58) |
| | Anemia | 21 (37) | 6 | (11) |
| | Thrombocytopenia | 14 (25) | 8 | (14) |
| Non-hematologic | Infections* | 29 (51) | 8 | (14) |
| AEs | Diarrhea | 17 (30) | 1 | (2) |
| | Constipation | 12 (21) | 0 | |
| | Rash | 12 (21) | 1 | (2) |

| | | |
|---|---|---|
| Pyrexia | 10 (18) | 0 |
| Peripheral neuropathy^{†} | 10 (18) | 0 |
| Asthenia | 9 (16) | 0 |

*Reported by System Organ Class (SOC);
^{†}Reported per standardized MedDRA query. AE, adverse event.

Grade 3-4 AEs were experienced by 43 patients (75%). The most common Grade 3-4 AEs were neutropenia (58%), thrombocytopenia (14%) and infections (14%). Twenty-two patients (39%) experienced a serious AE. Six patients (11I %) had Grade 5 AEs, one of which was treatment-related (neutropenic sepsis). AEs that led to treatment discontinuation, delay/interruption or dose reduction of any drug occurred in 6 (11%), 38 (67%) and 15 (26%) of patients, respectively.

AEs led to lenalidomide dose reduction or interruption in 25% and 63% of patients, respectively. One Grade 5 treatment-related AE or neutropenic sepsis was reported.

C. Efficacy

In total, 49 patients were treated at the RP2D of polatuzumab vedotin at a dose of 1.8 mg/kg and lenalidomide at a dose of 20 mg.

The PET-CR rate at EOI assessed by the IRC was 29%, based on the Modified Lugano criteria. A best overall response (BOR) assessed by investigator (INV) was seen in 36/49 (74%) patients with 17/49 (35%) patients achieving a CR. Of the patients that achieved CR, 14/17 (82%) remained in remission.

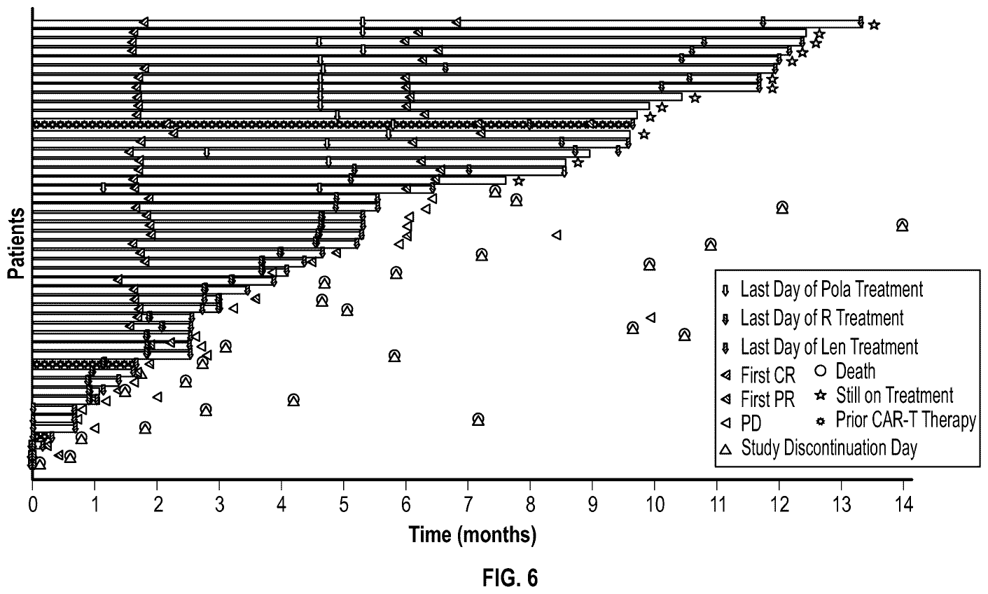

Durable responses were observed in patients treated with Pola-R-Len (FIG. 6).

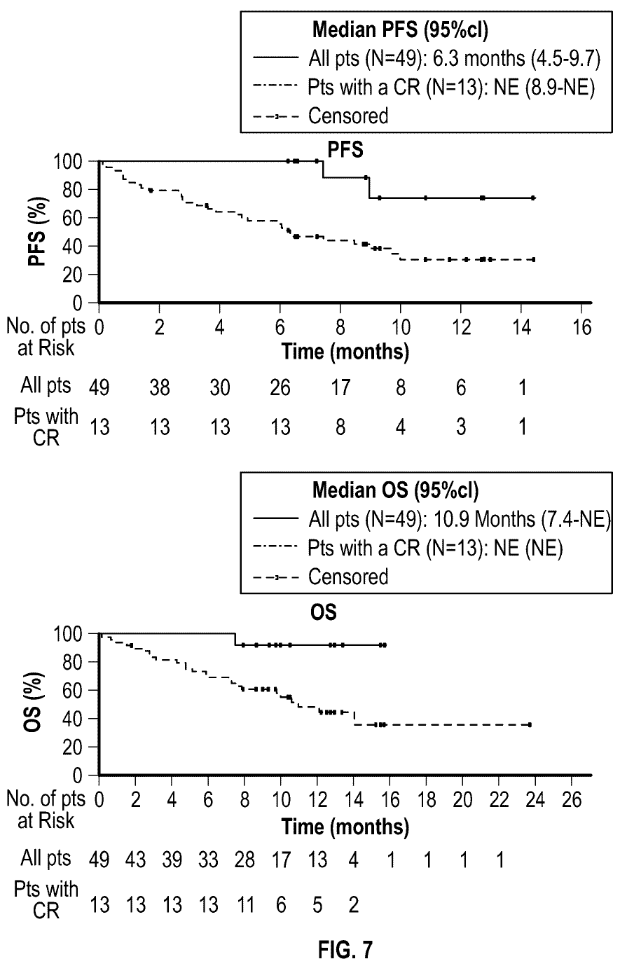

Kaplan-Meier survival curves for progression-free survival (PFS) and overall survival (OS) are shown in FIG. 7. Of the 13 patients that achieved a CR at the EOI (investigator-assessed), 11 (85%) remained in remission.

The median duration of response was 8.1 months (95% confidence interval [CI]: 4.7-not evaluable [NE]). After a median follow-up time of 9.5 months (range 0.1-23.7), the median progression free survival (PFS) and overall survival (OS) were 6.3 months (95% CI: 4.5-9.7) and 10.9 months (95% CI: 7.4-NE), respectively.

A summary of the efficacy results is provided in Table 17.

TABLE 17
Summary of Efficacy Results.

| | Efficacy Evaluable (N = 49) | | | |
|---|---|---|---|---|
| | Modified Lugano 2014* | | Lugano 2014 | |
| Response, n (%) | IRC | INV | IRC | INV |
| Objective response | 17 (35) | 19 (39) | 19 (39) | 19 (39) |
| Complete response | 14 (29) | 13 (27) | 14 (29) | 13 (27) |
| Partial response | 3 (6) | 6 (12) | 5 (10) | 6 (12) |
| Stable disease | 2 (4)^{†} | 0 | 0 | 0 |
| Disease progression | 15 (31) | 21 (43) | 15 (31) | 21 (43) |
| Missing/Not | 15 (31)^{‡¶} | 9 (18)^{¶} | 15 (31)^{‡¶} | 9 (18)^{¶} |
| evaluable | | | | |

INV-assessed

| | |
|---|---|
| Median DOR, months (95% CI) | 8.1 (4.7-NE) |
| Median PFS, months (95% CI) | 6.3 (4.5-9.7) |
| Median OS, months (95% CI) | 10.9 (7.4-NE) |

DOR, duration of response; EOI, end of induction; INV, Investigator assessed; IRC, Independent review committee assessed; NE, not evaluated; OS, overall survival; PFS, progression-free survival.
*Modified Lugano requires a negative bone marrow biopsy to confirm PET-CR and PET-PR must also meet CT-PR criteria;
^{†}Two cases of PET-PR were downgraded to SD by IRC due to Modified Lugano criteria;
^{‡}One patient assessed as CR by INV and was considered not evaluable by IRC;
^{¶}Two patients had CT-based CR but were unable to have PET scans performed due to COVID-19 restrictions; No responses were downgraded due to missing bone marrow biopsies.

Activity was observed in all cell of origin (COO) and double expressor lymphoma (DEL) subgroups, particularly in patients with activated B-cell (ABC) subtype (Table 18).

TABLE 18
Biomarker Analyses.

| | | | | BCL2 and MYC protein | | |
|---|---|---|---|---|---|---|
| | | COO by NanoString | | expression by IHC | | |
| | Total | GCB | ABC | BCL-2 | DEL | Non-DEL |
| Response | (N = 49) | (N = 27) | (N = 14) | (N = 14) | (N = 22) | (N = 23) |
| Objective response, | 17 (35) | 6 (22) | 5 (36) | 5 (36) | 6 (27) | 7 (30) |
| n (%) (IRC) | | | | | | |
| CR | 14 (29) | 3 (11) | 5 (36) | 4 (29) | 5 (23) | 5 (22) |
| PR | 3 (6) | 3 (11) | 0 (0) | 1 (7) | 1 (5) | 2 (9) |
| Median PFS, months | 6.3 | 3.58 | 7.0 | 6.3 | 5.4 | 6.3 |
| (95% CI) | (4.5-9.7) | (3.2-6.2) | (5.2-9.4) | (3.3-12.1) | (3.4-7.2) | (4.5-11.5) |
| Median OS, months | 10.9 | 9.2 | 11.3 | 9.8 | 9.3 | 9.8 |
| (95% CI) | (7.4-NE) | (6.2-10.4) | (8.0-12.5) | (5.7-14.3) | (7.0-11.8) | (6.8-13.5) |

ABC, activated B cell;
BCL-2, B-cell lymphoma 2;
CI, confidence interval;
COO, cell of origin;
CR, complete response;
DEL, double-expressor lymphoma;
GCB, germinal center B-cell;
IHC, immunohistochemistry;
IRC, independent review committee assessed;
NE, not evaluated;
OS, overall survival;
PFS, progression-free survival;
PR, partial response.

Subgroup analysis of patients achieving a CR (INV) revealed that patients with one prior line of therapy or who were non-refractory to last treatment were more likely to achieve CR at EOI (10/13 (77%) and 8/13 (62%) patients, respectively).

Among the 13 patients achieving a CR at EOI per INV, the median age was 75 years (range: 50-92), nine (69%) had International Prognostic Index (IPI) 3-5 at baseline, four (31%) had bulky disease (>7 cm), four (31%) had primary refractory disease, two (15%) received prior ASCT, and one (8%) received prior chimeric antigen receptor (CAR)-T cell therapy.

### III. CONCLUSIONS

The novel triplet combination, Pola-R-Len, demonstrated a tolerable safety profile. The safety profile of Pola-R-Len was consistent with the known profiles of the individual drugs. AEs were manageable with supportive care.

The efficacy results described in this Example show promising activity in a difficult-to-treat R/R DLBCL population, particularly in patients achieving CR at EOI, most of which remained in remission. Patients with non-refractory disease and those treated with only 1 prior line of therapy achieved higher CR rates compared to those with refractory disease or with multiple lines of prior treatment.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method for treating diffuse large B-cell lymphoma (DLBCL) in a human in need thereof, comprising administering to the human an effective amount of:
(a) an immunoconjugate comprising the formula: 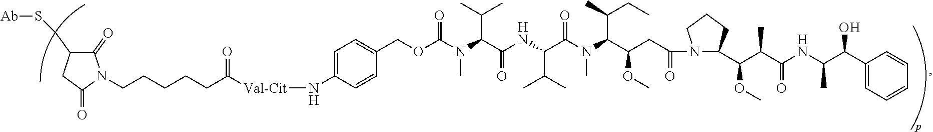
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) an immunomodulatory agent, and
(c) an anti-CD20 antibody; and
wherein the human achieves at least a complete response during or after treatment with the immunoconjugate, the immunomodulatory agent, and the anti-CD20 antibody.

2. The method of claim 1, wherein, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response during or after treatment with the immunoconjugate, the immunomodulatory agent, and the anti-CD20 antibody.

3. The method of claim 1 or claim 2, wherein, among a plurality of humans treated, at least about 70%, at least about 74%, at least about 80%, at least about 90%, or 100% of the humans achieve a best overall response during or after treatment with the immunoconjugate, the immunomodulatory agent, and the anti-CD20 antibody.

4. The method of any one of claims 1-3, wherein, among a plurality of humans treated, at least about 30%, at least about 35%, at least about 40%, at least about 50%, at least about 60%, at least about 70%, at least about 80%, at least about 90%, or 100% of the humans achieve a best complete response during or after treatment with the immunoconjugate, the immunomodulatory agent, and the anti-CD20 antibody.

5. The method of any one of claims 1-4, wherein, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response during or after treatment with the immunoconjugate, the immunomodulatory agent, and the anti-CD20 antibody.

6. The method of any one of claims 1-5, wherein the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response.

7. The method of any one of claims 1-6, wherein the human survives for at least about 4 months, at least about 5 months, at least about 6 months, at least about 7 months, at least about 8 months, at least about 9 months, at least about 10 months, or more, without disease progression, assessed from the start of treatment with the immunoconjugate, the immunomodulatory agent, and the anti-CD20 antibody.

8. The method of any one of claims 1-7, wherein the human survives for at least about 7 months, at least about 8 months, at least about 9 months, at least about 10 months, at least about 11 months, or more, assessed from the start of treatment with the immunoconjugate, the immunomodulatory agent, and the anti-CD20 antibody.

9. The method of any one of claims 1-8, wherein the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20.

10. The method of any one of claims 1-9, wherein the anti-CD79b antibody comprises (i) a heavy chain comprising the amino acid sequence of SEQ ID NO: 36 and (ii) a light chain comprising the amino acid sequence of SEQ ID NO: 35.

11. The method of any one of claims 1-10, wherein the immunoconjugate is polatuzumab vedotin.

12. The method of any one of claims 1-11, wherein the immunomodulatory agent is lenalidomide.

13. The method of any one of claims 1-12, wherein the anti-CD20 antibody is rituximab.

14. The method of claim 13, wherein the polatuzumab vedotin is administered at a dose of about 1.8 mg/kg, the lenalidomide is administered at a dose between about 10 mg and about 20 mg, and the rituximab is administered at a dose of about 375 mg/m².

15. The method of claim 14, wherein the polatuzumab vedotin, the lenalidomide, and the rituximab are administered during an induction phase in 28-day cycles, wherein:
the polatuzumab vedotin is administered intravenously at a dose of about 1.8 mg/kg on Day 1 of each 28-day cycle,
the lenalidomide is administered orally at a dose between about 10 mg and about 20 mg on each of Days 1-21 of each 28-day cycle, and
the rituximab is administered intravenously at a dose of about 375 mg/m² on Day 1 of each 28-day cycle;
optionally, wherein the induction phase comprises at least six 28-day cycles.

16. The method of claim 15, wherein the polatuzumab vedotin, the lenalidomide, and the rituximab are administered sequentially.

17. The method of claim 16, wherein the lenalidomide is administered prior to the rituximab and the rituximab is administered prior to the polatuzumab vedotin on Day 1 of each 28-day cycle.

18. The method of any one of claims 15-17, wherein, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response after six 28-day cycles.

19. The method of any one of claims 15-18, wherein, among a plurality of humans treated, at least about 70%, at least about 74%, at least about 80%, at least about 90%, or 100% of the humans achieve a best overall response after six 28-day cycles.

20. The method of any one of claims 15-19, wherein, among a plurality of humans treated, at least about 30%, at least about 35%, at least about 40%, at least about 50%, at least about 60%, at least about 70%, at least about 80%, at least about 90%, or 100% of the humans achieve a best complete response after six 28-day cycles.

21. The method of any one of claims 15-20, wherein, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response after six 28-day cycles.

22. The method of any one of claims 18-21, wherein the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response.

23. The method of any one of claims 15-22, wherein the human survives for at least about 4 months, at least about 5 months, at least about 6 months, at least about 7 months, at least about 8 months, at least about 9 months, at least about 10 months, or more, without disease progression, assessed from the start of treatment with the polatuzumab vedotin, the lenalidomide, and the rituximab.

24. The method of any one of claims 15-23, wherein the human survives for at least about 7 months, at least about 8 months, at least about 9 months, at least about 10 months, at least about 11 months, or more, assessed from the start of treatment with the polatuzumab vedotin, the lenalidomide, and the rituximab.

25. The method of any one of claims 15-24, wherein the lenalidomide and the rituximab are further administered during a consolidation phase after the sixth 28-day cycle of the induction phase.

26. The method of claim 25, wherein:
the lenalidomide is administered orally at a dose of about 10 mg on each of Days 1-21 of each month during the consolidation phase, and
the rituximab is administered intravenously at a dose of about 375 mg/m² on Day 1 of every other month during the consolidation phase.

27. The method of claim 26, wherein the lenalidomide is administered for a maximum of 6 months during the consolidation phase.

28. The method of claim 26 or claim 27, wherein the rituximab is administered on Day 1 of each of the first, third, and fifth months during the consolidation phase.

29. The method of any one of claims 25-28, wherein the lenalidomide and the rituximab are administered sequentially during the consolidation phase.

30. The method of claim 29, wherein the lenalidomide is administered prior to the rituximab on Day 1 of each of the first, third, and fifth months during the consolidation phase.

31. A method for treating diffuse large B-cell lymphoma (DLBCL) in a human in need thereof, comprising administering to the human an effective amount of:
(a) an immunoconjugate comprising the formula: 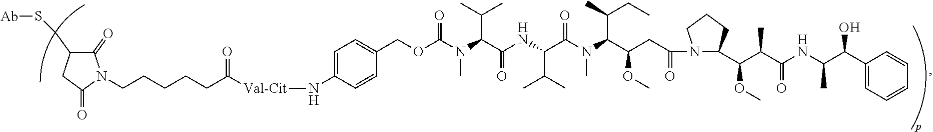
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) an immunomodulatory agent, and
(c) an anti-CD20 antibody; and
wherein the human does not demonstrate disease progression within at least about 4 months after the start of treatment with the immunoconjugate, the immunomodulatory agent and the anti-CD20 antibody.

32. The method of claim 31, wherein, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response during or after treatment with the immunoconjugate, the immunomodulatory agent, and the anti-CD20 antibody.

33. The method of claim 31 or claim 32, wherein, among a plurality of humans treated, at least about 70%, at least about 74%, at least about 80%, at least about 90%, or 100% of the humans achieve a best overall response during or after treatment with the immunoconjugate, the immunomodulatory agent, and the anti-CD20 antibody.

34. The method of any one of claims 31-33, wherein, among a plurality of humans treated, at least about 30%, at least about 35%, at least about 40%, at least about 50%, at least about 60%, at least about 70%, at least about 80%, at least about 90%, or 100% of the humans achieve a best complete response during or after treatment with the immunoconjugate, the immunomodulatory agent, and the anti-CD20 antibody.

35. The method of any one of claims 31-34, wherein, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response during or after treatment with the immunoconjugate, the immunomodulatory agent, and the anti-CD20 antibody.

36. The method of any one of claims 32-35, wherein the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response.

37. The method of any one of claims 31-36, wherein the human survives for at least about 4 months, at least about 5 months, at least about 6 months, at least about 7 months, at least about 8 months, at least about 9 months, at least about 10 months, or more, without disease progression, assessed from the start of treatment with the immunoconjugate, the immunomodulatory agent, and the anti-CD20 antibody.

38. The method of any one of claims 31-37, wherein the human survives for at least about 7 months, at least about 8 months, at least about 9 months, at least about 10 months, at least about 11 months, or more, assessed from the start of treatment with the immunoconjugate, the immunomodulatory agent, and the anti-CD20 antibody.

39. The method of any one of claims 31-38, wherein the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20.

40. The method of any one of claims 31-39, wherein the anti-CD79b antibody comprises (i) a heavy chain comprising the amino acid sequence of SEQ ID NO: 36 and (ii) a light chain comprising the amino acid sequence of SEQ ID NO: 35.

41. The method of any one of claims 31-40, wherein the immunoconjugate is polatuzumab vedotin.

42. The method of any one of claims 31-41, wherein the immunomodulatory agent is lenalidomide.

43. The method of any one of claims 31-42, wherein the anti-CD20 antibody is rituximab.

44. The method of claim 43, wherein the polatuzumab vedotin is administered at a dose of about 1.8 mg/kg, the lenalidomide is administered at a dose between about 10 mg and about 20 mg, and the rituximab is administered at a dose of about 375 mg/m².

45. The method of claim 44, wherein the polatuzumab vedotin, the lenalidomide, and the rituximab are administered during an induction phase in 28-day cycles, wherein:
the polatuzumab vedotin is administered intravenously at a dose of about 1.8 mg/kg on Day 1 of each 28-day cycle,
the lenalidomide is administered orally at a dose between about 10 mg and about 20 mg on each of Days 1-21 of each 28-day cycle, and
the rituximab is administered intravenously at a dose of about 375 mg/m² on Day 1 of each 28-day cycle;
optionally, wherein the induction phase comprises at least six 28-day cycles.

46. The method of claim 45, wherein the polatuzumab vedotin, the lenalidomide, and the rituximab are administered sequentially.

47. The method of claim 46, wherein the lenalidomide is administered prior to the rituximab and the rituximab is administered prior to the polatuzumab vedotin on Day 1 of each 28-day cycle.

48. The method of any one of claims 45-47, wherein, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response after six 28-day cycles.

49. The method of any one of claims 45-48, wherein, among a plurality of humans treated, at least about 70%, at least about 74%, at least about 80%, at least about 90%, or 100% of the humans achieve a best overall response after six 28-day cycles.

50. The method of any one of claims 45-49, wherein, among a plurality of humans treated, at least about 30%, at least about 35%, at least about 40%, at least about 50%, at least about 60%, at least about 70%, at least about 80%, at least about 90%, or 100% of the humans achieve a best complete response after six 28-day cycles.

51. The method of any one of claims 45-50, wherein, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response after six 28-day cycles.

52. The method of any one of claims 48-51, wherein the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response.

53. The method of any one of claims 45-52, wherein the human survives for at least about 4 months, at least about 5 months, at least about 6 months, at least about 7 months, at least about 8 months, at least about 9 months, at least about 10 months, or more, without disease progression, assessed from the start of treatment with the polatuzumab vedotin, the lenalidomide, and the rituximab.

54. The method of any one of claims 45-53, wherein the human survives for at least about 7 months, at least about 8 months, at least about 9 months, at least about 10 months, at least about 11 months, or more, assessed from the start of treatment with the polatuzumab vedotin, the lenalidomide, and the rituximab.

55. The method of any one of claims 45-54, wherein the lenalidomide and the rituximab are further administered during a consolidation phase after the sixth 28-day cycle of the induction phase.

56. The method of claim 55, wherein:
the lenalidomide is administered orally at a dose of about 10 mg on each of Days 1-21 of each month during the consolidation phase, and
the rituximab is administered intravenously at a dose of about 375 mg/m² on Day 1 of every other month during the consolidation phase.

57. The method of claim 56, wherein the lenalidomide is administered for a maximum of 6 months during the consolidation phase.

58. The method of claim 56 or 57, wherein the rituximab is administered on Day 1 of each of the first, third, and fifth months during the consolidation phase.

59. The method of any one of claims 55-58, wherein the lenalidomide and the rituximab are administered sequentially during the consolidation phase.

60. The method of claim 59, wherein the lenalidomide is administered prior to the rituximab on Day 1 of each of the first, third, and fifth months during the consolidation phase.

61. A method of treating diffuse large B-cell lymphoma (DLBCL) in a human in need thereof, comprising administering to the human an effective amount of:
(a) an immunoconjugate comprising the formula: 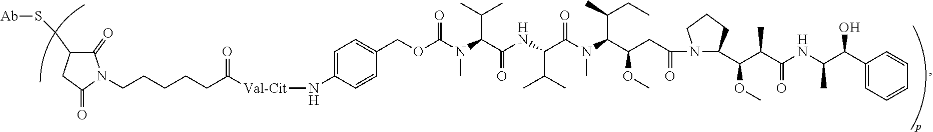
wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and
wherein p is between 2 and 5,
(b) lenalidomide and
(c) rituximab,
wherein the immunoconjugate is administered at a dose of about 1.8 mg/kg, the lenalidomide is administered at a dose between about 10 mg and about 20 mg, and the rituximab is administered at a dose of about 375 mg/m², and
wherein the human achieves at least a complete response during or after treatment with the immunoconjugate, the lenalidomide, and the rituximab.

62. The method of claim 61, wherein, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response during or after treatment with the immunoconjugate, the lenalidomide, and the rituximab.

63. The method of claim 61 or claim 62, wherein, among a plurality of humans treated, at least about 70%, at least about 74%, at least about 80%, at least about 90%, or 100% of the humans achieve a best overall response during or after treatment with the immunoconjugate, the lenalidomide, and the rituximab.

64. The method of any one of claims 61-63, wherein, among a plurality of humans treated, at least about 30%, at least about 35%, at least about 40%, at least about 50%, at least about 60%, at least about 70%, at least about 80%, at least about 90%, or 100% of the humans achieve a best complete response during or after treatment with the immunoconjugate, the lenalidomide, and the rituximab.

65. The method of any one of claims 61-64, wherein, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response during or after treatment with the immunoconjugate, the lenalidomide, and the rituximab.

66. The method of any one of claims 61-65, wherein the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response.

67. The method of any one of claims 61-66, wherein the human survives for at least about 4 months, at least about 5 months, at least about 6 months, at least about 7 months, at least about 8 months, at least about 9 months, at least about 10 months, or more, without disease progression, assessed from the start of treatment with the immunoconjugate, the lenalidomide, and the rituximab.

68. The method of any one of claims 61-67, wherein the human survives for at least about 7 months, at least about 8 months, at least about 9 months, at least about 10 months, at least about 11 months, or more, assessed from the start of treatment with the immunoconjugate, the lenalidomide, and the rituximab.

69. The method of any one of claims 61-68, wherein p is between 3 and 4.

70. The method of any one of claims 61-69, wherein the antibody comprises (i) a heavy chain comprising the amino acid sequence of SEQ ID NO: 36 and (ii) a light chain comprising the amino acid sequence of SEQ ID NO: 35.

71. The method of any one of claims 61-70, wherein the immunoconjugate is polatuzumab vedotin.

72. The method of claim 71, wherein the polatuzumab vedotin, the lenalidomide, and the rituximab are administered during an induction phase in 28-day cycles, wherein:
the polatuzumab vedotin is administered intravenously at a dose of about 1.8 mg/kg on Day 1 of each 28-day cycle,
the lenalidomide is administered orally at a dose between about 10 mg and about 20 mg on each of Days 1-21 of each 28-day cycle, and
the rituximab is administered intravenously at a dose of about 375 mg/m² on Day 1 of each 28-day cycle;
optionally, wherein the induction phase comprises at least six 28-day cycles.

73. The method of claim 72, wherein the polatuzumab vedotin, the lenalidomide, and the rituximab are administered sequentially.

74. The method of claim 73, wherein the lenalidomide is administered prior to the rituximab and the rituximab is administered prior to the polatuzumab vedotin on Day 1 of each 28-day cycle.

75. The method of any one of claims 72-74, wherein, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response after six 28-day cycles.

76. The method of any one of claims 72-75, wherein, among a plurality of humans treated, at least about 70%, at least about 74%, at least about 80%, at least about 90%, or 100% of the humans achieve a best overall response after six 28-day cycles.

77. The method of any one of claims 72-76, wherein, among a plurality of humans treated, at least about 30%, at least about 35%, at least about 40%, at least about 50%, at least about 60%, at least about 70%, at least about 80%, at least about 90%, or 100% of the humans achieve a best complete response after six 28-day cycles.

78. The method of any one of claims 72-77, wherein, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response after six 28-day cycles.

79. The method of any one of claims 75-78, wherein the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response.

80. The method of any one of claims 72-79, wherein the human survives for at least about 4 months, at least about 5 months, at least about 6 months, at least about 7 months, at least about 8 months, at least about 9 months, at least about 10 months, or more, without disease progression, assessed from the start of treatment with the polatuzumab vedotin, the lenalidomide, and the rituximab.

81. The method of any one of claims 72-80, wherein the human survives for at least about 7 months, at least about 8 months, at least about 9 months, at least about 10 months, at least about 11 months, or more, assessed from the start of treatment with the polatuzumab vedotin, the lenalidomide, and the rituximab.

82. The method of claim 72-81, wherein the lenalidomide and the rituximab are further administered during a consolidation phase after the sixth 28-day cycle of the induction phase.

83. The method of claim 82, wherein:
the lenalidomide is administered orally at a dose of about 10 mg on each of Days 1-21 of each month during the consolidation phase, and
the rituximab is administered intravenously at a dose of about 375 mg/m² on Day 1 of every other month during the consolidation phase.

84. The method of claim 83, wherein the lenalidomide is administered for a maximum of 6 months during the consolidation phase.

85. The method of claim 83 or claim 84, wherein the rituximab is administered on Day 1 of each of the first, third, and fifth months during the consolidation phase.

86. The method of any one of claims 82-85, wherein the lenalidomide and the rituximab are administered sequentially during the consolidation phase.

87. The method of claim 86, wherein the lenalidomide is administered prior to the rituximab on Day 1 of each of the first, third, and fifth months during the consolidation phase.

88. A method of treating diffuse large B-cell lymphoma (DLBCL) in a human in need thereof, comprising administering to the human an effective amount of:
(a) polatuzumab vedotin;
(b) lenalidomide; and
(c) rituximab,
during an induction phase in 28-day cycles,
wherein, during the induction phase, the polatuzumab vedotin is administered at a dose of about 1.8 mg/kg, the lenalidomide is administered at a dose of about 20 mg, and the rituximab is administered at a dose of about 375 mg/m², and
wherein the human achieves a complete response during or after the induction phase.

89. The method of claim 88, wherein the induction phase comprises at least six 28-day cycles.

90. The method of claim 88 or claim 89, wherein:
the polatuzumab vedotin is administered intravenously at a dose of about 1.8 mg/kg on Day 1 of each 28-day cycle,
the lenalidomide is administered orally at a dose of about 20 mg on each of Days 1-21 of each 28-day cycle, and
the rituximab is administered intravenously at a dose of about 375 mg/m² on Day 1 of each 28-day cycle.

91. The method of claim 90, wherein the polatuzumab vedotin, the lenalidomide, and the rituximab are administered sequentially.

92. The method of claim 91, wherein the lenalidomide is administered prior to the rituximab and the rituximab is administered prior to the polatuzumab vedotin on Day 1 of each 28-day cycle.

93. The method of any one of claims 88-92, wherein, among a plurality of humans treated, at least about 25%, at least about 27%, at least about 29%, at least about 30%, at least about 40%, at least about 50%, at least about 60%, at least about 70%, at least about 80%, at least about 90%, or 100% of the humans achieve a complete response after six 28-day cycles.

94. The method of any one of claims 88-93, wherein, among a plurality of humans treated, at least about 70%, at least about 74%, at least about 80%, at least about 90%, or 100% of the humans achieve a best overall response after six 28-day cycles.

95. The method of any one of claims 88-94, wherein, among a plurality of humans treated, at least about 30%, at least about 35%, at least about 40%, at least about 50%, at least about 60%, at least about 70%, at least about 80%, at least about 90%, or 100% of the humans achieve a best complete response after six 28-day cycles.

96. The method of any one of claims 88-95, wherein, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response after six 28-day cycles.

97. The method of any one of claims 88-96, wherein the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response.

98. The method of any one of claims 88-97, wherein the human survives for at least about 4 months, at least about 5 months, at least about 6 months, at least about 7 months, at least about 8 months, at least about 9 months, at least about 10 months, or more, without disease progression, assessed from the start of treatment with the polatuzumab vedotin, the lenalidomide, and the rituximab.

99. The method of any one of claims 88-98, wherein the human survives for at least about 7 months, at least about 8 months, at least about 9 months, at least about 10 months, at least about 11 months, or more, assessed from the start of treatment with the polatuzumab vedotin, the lenalidomide, and the rituximab.

100. The method of any one of claims 88-99, wherein the induction phase is followed by a consolidation phase, wherein the lenalidomide is administered at a dose of about 10 mg and the rituximab is administered at a dose of about 375 mg/m² during the consolidation phase.

101. The method of claim 100, wherein:
the lenalidomide is administered orally at a dose of about 10 mg on each of Days 1-21 of each month during the consolidation phase, and
the rituximab is administered intravenously at a dose of about 375 mg/m² on Day 1 of every other month during the consolidation phase.

102. The method of claim 100 or claim 101, wherein the lenalidomide is administered for a maximum of 6 months during the consolidation phase.

103. The method of any one of claims 100-102, wherein the rituximab is administered on Day 1 of each of the first, third, and fifth months during the consolidation phase.

104. The method of any one of claims 100-103, wherein the lenalidomide and the rituximab are administered sequentially during the consolidation phase.

105. The method of claim 104, wherein the lenalidomide is administered prior to the rituximab on Day 1 of each of the first, third, and fifth months during the consolidation phase.

106. A method of treating diffuse large B-cell lymphoma (DLBCL) in a plurality of humans in need thereof, comprising administering to the humans an effective amount of:
(a) polatuzumab vedotin;
(b) lenalidomide; and
(c) rituximab,
during an induction phase in 28-day cycles,
wherein, during the induction phase, the polatuzumab vedotin is administered at a dose of about 1.8 mg/kg, the lenalidomide is administered at a dose of about 20 mg, and the rituximab is administered at a dose of about 375 mg/m², and
wherein, at least about 25% of the humans in the plurality achieve a complete response during or after the induction phase.

107. The method of claim 106, wherein the induction phase comprises at least six 28-day cycles.

108. The method of claim 106 or claim 107, wherein:
the polatuzumab vedotin is administered intravenously at a dose of about 1.8 mg/kg on Day 1 of each 28-day cycle,
the lenalidomide is administered orally at a dose of about 20 mg on each of Days 1-21 of each 28-day cycle, and
the rituximab is administered intravenously at a dose of about 375 mg/m² on Day 1 of each 28-day cycle.

109. The method of any one of claims 106-108, wherein the polatuzumab vedotin, the lenalidomide, and the rituximab are administered sequentially.

110. The method of claim 109, wherein the lenalidomide is administered prior to the rituximab and the rituximab is administered prior to the polatuzumab vedotin on Day 1 of each 28-day cycle.

111. The method of any one of claims 106-110, wherein at least about 27%, at least about 29%, at least about 30%, at least about 40%, at least about 50%, at least about 60%, at least about 70%, at least about 80%, at least about 90%, or 100% of the humans in the plurality achieve a complete response after six 28-day cycles.

112. The method of any one of claims 106-111, wherein at least about 70%, at least about 74%, at least about 80%, at least about 90%, or 100% of the humans in the plurality achieve a best overall response after six 28-day cycles.

113. The method of any one of claims 106-112, wherein at least about 30%, at least about 35%, at least about 40%, at least about 50%, at least about 60%, at least about 70%, at least about 80%, at least about 90%, or 100% of the humans in the plurality achieve a best complete response after six 28-day cycles.

114. The method of any one of claims 106-113, wherein, among a plurality of humans treated, at least about 30%, at least about 35%, at least about 39%, at least about 40%, at least about 50%, at least about 60%, at least about 70%, at least about 80%, at least about 90%, or 100% of the humans achieve an objective response after six 28-day cycles.

115. The method of any one of claims 106-114, wherein the duration of the complete response, best complete response, objective response, or best overall response is at least about 4 months, at least about 5 months, at least about 6 months, at least about 7 months, at least about 8 months, at least about 9 months, or more, assessed from the time of the first occurrence of the complete response, best complete response, objective response, or best overall response.

116. The method of any one of claims 106-115, wherein the induction phase is followed by a consolidation phase, wherein the lenalidomide is administered at a dose of about 10 mg and the rituximab is administered at a dose of about 375 mg/m² during the consolidation phase.

117. The method of claim 116, wherein:
the lenalidomide is administered orally at a dose of about 10 mg on each of Days 1-21 of each month during the consolidation phase, and
the rituximab is administered intravenously at a dose of about 375 mg/m² on Day 1 of every other month during the consolidation phase.

118. The method claim 116 or claim 117, wherein the lenalidomide is administered for a maximum of 6 months during the consolidation phase.

119. The method of any one of claims 116-118, wherein the rituximab is administered on Day 1 of each of the first, third, and fifth months during the consolidation phase.

120. The method of any one of claims 116-119, wherein the lenalidomide and the rituximab are administered sequentially during the consolidation phase.

121. The method of claim 120, wherein the lenalidomide is administered prior to the rituximab on Day 1 of each of the first, third, and fifth months during the consolidation phase.

122. The method of any one of claims 1-121, wherein the human or a human in the plurality of humans has received at least one prior therapy for DLBCL.

123. The method of any one of claims 1-122, wherein the human or a human in the plurality of humans has received at least two prior therapies for DLBCL.

124. The method of any one of claims 1-123, wherein the human or a human in the plurality of humans has received a prior therapy for DLBCL comprising a chemoimmunotherapy that included an anti-CD20 antibody.

125. The method of any one of claims 1-124, wherein the human or a human in the plurality of humans has been administered a prior bone marrow transplant for DLBCL.

126. The method of any one of claims 1-125, wherein the human or a human in the plurality of humans has been administered a prior chimeric antigen receptor (CAR)-T-cell therapy for DLBCL.

127. The method of any one of claims 122-126, wherein the human or a human in the plurality of humans has DLBCL that was refractory to the first prior treatment for DLBCL administered to the human or the human in the plurality of humans.

128. The method of any one of claims 122-127, wherein the human or a human in the plurality of humans has DLBCL that was refractory to the most recent prior therapy for DLBCL.

129. The method of any one of claims 1-128, wherein the DLBCL is relapsed/refractory DLBCL.

130. The method of any one of claims 1-129, wherein the DLBCL is relapsed/refractory DLBCL after treatment with at least one prior chemoimmunotherapy regimen that included an anti-CD20 antibody.

131. The method of any one of claims 122-130, wherein the human or a human in the plurality of humans experienced disease progression after treatment with high-dose chemotherapy and autologous stem-cell transplantation.

132. The method of any one of claims 1-131, wherein the DLBCL is CD20-positive DLBCL.

133. The method of any one of claims 1-132, wherein the DLBCL is a positron emission tomography (PET)-positive lymphoma.

134. The method of any one of claims 1-133, wherein the human or a human in the plurality of humans is not eligible for autologous stem-cell transplantation.

135. The method of any one of claims 1-134, wherein the human or a human in the plurality of humans does not have central nervous system (CNS) lymphoma or leptomeningeal infiltration.

136. The method of any one of claims 1-135, wherein the human or a human in the plurality of humans has at least one bi-dimensionally measurable lesion.

137. The method of claim 136, wherein the at least one bi-dimensionally measurable lesion is greater than 1.5 cm in its largest dimension, assessed by computed tomography (CT) scan or magnetic resonance imaging (MRI).

138. The method of any one of claims 1-137, wherein the human or a human in the plurality of humans has not received a prior allogenic stem cell transplantation (SCT).

139. The method of any one of claims 1-138, wherein the human or a human in the plurality of humans does not have history of transformation of indolent disease to DLBCL.

140. The method of any one of claims 1-139, wherein the human or a human in the plurality of humans does not have Grade 2 or greater neuropathy.

141. The method of any one of claims 1-139, wherein the human or a human in the plurality of humans has an Eastern Cooperative Oncology Group (ECOG) Performance Status of 0, 1, or 2.

142. The method of any one of claims 1-141, wherein the human or a human in the plurality of humans has DLBCL with an Ann Arbor Stage III or IV.

143. The method of any one of claims 1-142, wherein the human or a human in the plurality of humans has DLBCL with an International Prognostic Index of between 3 and 5.

144. A kit comprising an immunoconjugate comprising the formula: 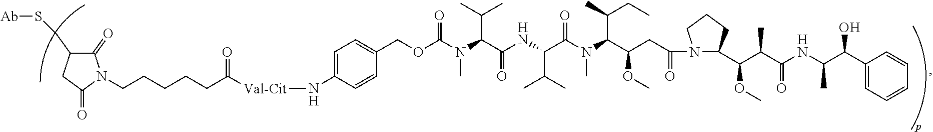
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and
wherein p is between 1 and 8,
for use in combination with an immunomodulatory agent and an anti-CD20 antibody for treating a human in need thereof having diffuse large B-cell lymphoma (DLBCL) according to a method of any one of claims 1-60 and 122-143.

145. A kit comprising an immunoconjugate comprising the formula: 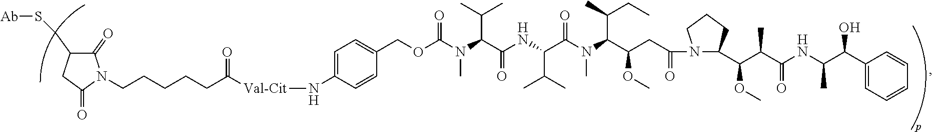
wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and
wherein p is between 2 and 5,
for use in combination with lenalidomide and rituximab for treating a human in need thereof having diffuse large B-cell lymphoma (DLBCL) according to the method of any one of claims 61-88 and 122-143.

146. The kit of claim 144 or claim 145, wherein p is between 3 and 4.

147. The kit of any one of claims 144-146, wherein the antibody comprises (i) a heavy chain comprising the amino acid sequence of SEQ ID NO: 36 and (ii) a light chain comprising the amino acid sequence of SEQ ID NO: 35.

148. A kit comprising polatuzumab vedotin for use in combination with lenalidomide and rituximab for treating a human in need thereof having diffuse large B-cell lymphoma (DLBCL) according to the method of any one of claims 88-143.

149. The kit of any one of claims 144-148, wherein the DLBCL is relapsed/refractory DLBCL.

150. An immunoconjugate comprising the formula: 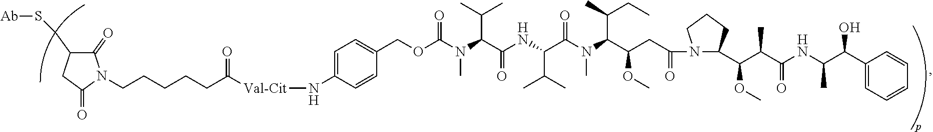
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and
wherein p is between 1 and 8,
for use in a method of treating diffuse large B-cell lymphoma (DLBCL) according to any one of claims 1-60 and 122-143.

151. The immunoconjugate of claim 150, wherein the anti-CD79b antibody comprises (i) a heavy chain variable domain (VH) that comprises the amino acid sequence of SEQ ID NO: 19 and (ii) a light chain variable domain (VL) that comprises the amino acid sequence of SEQ ID NO: 20.

152. An immunoconjugate comprising the formula: 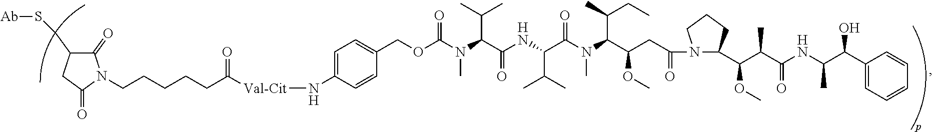
wherein Ab is an anti-CD79b antibody that comprises (i) a heavy chain variable domain (VH) comprising the amino acid sequence of SEQ ID NO: 19 and (ii) a light chain variable domain (VL) comprising the amino acid sequence of SEQ ID NO: 20, and
wherein p is between 2 and 5,
for use in a method of treating diffuse large B-cell lymphoma (DLBCL) according to any one of claims 61-88 and 122-143.

153. The immunoconjugate of any one of claims 150-152, wherein p is between 3 and 4.

154. The immunoconjugate of any one of claims 150-153, wherein the anti-CD79b antibody comprises (i) a heavy chain comprising the amino acid sequence of SEQ ID NO: 36 and (ii) a light chain comprising the amino acid sequence of SEQ ID NO: 35.

155. Polatuzumab vedotin for use in a method of treating diffuse large B-cell lymphoma (DLBCL) according to any one of claims 88-143.

156. The immunoconjugate for use according to any one of claims 150-154, or the polatuzumab vedotin for use according to claim 155, wherein the DLBCL is relapsed/refractory DLBCL.


SEQUENCE LISTING (56 sequences)

SEQ ID NO: 1 - protein, 179 aa, Homo sapiens
RFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 2 - protein, 201 aa, Homo sapiens
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 3 - protein, 112 aa, synthetic construct
  REGION 1..112: note = Synthetic Construct
GPELVKPGASVKISCKASGYAFSYSWMNWVKLRPGQGLEWIGRIFPGDGDTDYNGKFKGKATLTADKSSNTAYMQLTSLTSVDSAVYLCARNVFDGYWLVYWGQGTLVTVSA

SEQ ID NO: 4 - protein, 103 aa, synthetic construct
  REGION 1..103: note = Synthetic Construct
NPVTLGTSASISCRSSKSLLHSNGITYLYWYLQKPGQSPQLLIYQMSNLVSGVPDRFSSSGSGTDFTLRISRVEAEDVGVYYCAQNLELPYTFGGGTKLEIKR

SEQ ID NO: 5 - protein, 6 aa, synthetic construct
  REGION 1..6: note = Synthetic Construct
GYAFSY

SEQ ID NO: 6 - protein, 8 aa, synthetic construct
  REGION 1..8: note = Synthetic Construct
FPGDGDTD

SEQ ID NO: 7 - protein, 10 aa, synthetic construct
  REGION 1..10: note = Synthetic Construct
NVFDGYWLVY

SEQ ID NO: 8 - protein, 16 aa, synthetic construct
  REGION 1..16: note = Synthetic Construct
RSSKSLLHSNGITYLY

SEQ ID NO: 9 - protein, 7 aa, synthetic construct
  REGION 1..7: note = Synthetic Construct
QMSNLVS

SEQ ID NO: 10 - protein, 9 aa, synthetic construct
  REGION 1..9: note = Synthetic Construct
AQNLELPYT

SEQ ID NO: 11 - protein, 119 aa, synthetic construct
  REGION 1..119: note = Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 12 - protein, 115 aa, synthetic construct
  REGION 1..115: note = Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 13 - protein, 448 aa, synthetic construct
  REGION 1..448: note = Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 219 aa, synthetic construct
  REGION 1..219: note = Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 119 aa, synthetic construct
  REGION 1..119: note = Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 119 aa, synthetic construct
  REGION 1..119: note = Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYLCARNVFDGYWLVYWGQGTLVTVSS

SEQ ID NO: 17 - protein, 448 aa, synthetic construct; identical to SEQ ID NO: 13
  REGION 1..448: note = Synthetic Construct

SEQ ID NO: 18 - protein, 219 aa, synthetic construct; identical to SEQ ID NO: 14
  REGION 1..219: note = Synthetic Construct

SEQ ID NO: 19 - protein, 117 aa, Homo sapiens [sequence omitted]

SEQ ID NO: 20 - protein, 112 aa, Homo sapiens
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 21 - protein, 10 aa, synthetic construct
  REGION 1..10: note = Synthetic Construct
GYTFSSYWIE

SEQ ID NO: 22 - protein, 18 aa, synthetic construct
  REGION 1..18: note = Synthetic Construct
GEILPGGGDTNYNEIFKG

SEQ ID NO: 23 - protein, 10 aa, synthetic construct
  REGION 1..10: note = Synthetic Construct
TRRVPIRLDY

SEQ ID NO: 24 - protein, 15 aa, synthetic construct
  REGION 1..15: note = Synthetic Construct
KASQSVDYEGDSFLN

SEQ ID NO: 25 - protein, 7 aa, synthetic construct
  REGION 1..7: note = Synthetic Construct
AASNLES

SEQ ID NO: 26 - protein, 9 aa, synthetic construct
  REGION 1..9: note = Synthetic Construct
QQSNEDPLT

SEQ ID NO: 27 - protein, 25 aa, synthetic construct [sequence omitted]

SEQ ID NO: 28 - protein, 13 aa, synthetic construct
  REGION 1..13: note = Synthetic Construct
WVRQAPGKGLEWI

SEQ ID NO: 29 - protein, 30 aa, synthetic construct
  REGION 1..30: note = Synthetic Construct
RATFSADTSKNTAYLQMNSLRAEDTAVYYC

SEQ ID NO: 30 - protein, 11 aa, synthetic construct
  REGION 1..11: note = Synthetic Construct
WGQGTLVTVSS

SEQ ID NO: 31 - protein, 23 aa, synthetic construct
  REGION 1..23: note = Synthetic Construct
DIQLTQSPSSLSASVGDRVTITC

SEQ ID NO: 32 - protein, 15 aa, synthetic construct
  REGION 1..15: note = Synthetic Construct
WYQQKPGKAPKLLIY

SEQ ID NO: 33 - protein, 32 aa, synthetic construct
  REGION 1..32: note = Synthetic Construct
GVPSRFSGSGSGTDFTLTISSLQPEDFATYYC

SEQ ID NO: 34 - protein, 11 aa, synthetic construct
  REGION 1..11: note = Synthetic Construct
FGQGTKVEIKR

SEQ ID NO: 35 - protein, 218 aa, synthetic construct
  REGION 1..218: note = Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 446 aa, synthetic construct
  REGION 1..446: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 446 aa, synthetic construct
  REGION 1..446: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 218 aa, synthetic construct
  REGION 1..218: note = Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 39 - protein, 447 aa, synthetic construct
  REGION 1..447: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 40 - protein, 119 aa, synthetic construct
  REGION 1..119: note = Synthetic Construct
QVQLVQSGAEVKKPGASVKVSCKVSGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 41 - protein, 119 aa, synthetic construct
  REGION 1..119: note = Synthetic Construct
QVQLVQSGAEVKKPGSSVKVSCKASGYAFSYSWMSWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 42 - protein, 119 aa, synthetic construct; identical to SEQ ID NO: 11
  REGION 1..119: note = Synthetic Construct

SEQ ID NO: 43 - protein, 119 aa, synthetic construct
  REGION 1..119: note = Synthetic Construct
QVQLVQSGAEVKKPGSSVKVSCKASGYAFSYSWISWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 44 - protein, 119 aa, synthetic construct
  REGION 1..119: note = Synthetic Construct
QVQLVQSGAEVKKPGASVKVSCKASGYTFTYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 45 - protein, 119 aa, synthetic construct
  REGION 1..119: note = Synthetic Construct
QVQLVQSGAEVKKPGASVKVSCKASGYTFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 46 - protein, 119 aa, synthetic construct
  REGION 1..119: note = Synthetic Construct
EVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 47 - protein, 119 aa, synthetic construct
  REGION 1..119: note = Synthetic Construct
EVQLVESGGGLVKPGGSLRLSCAASGFAFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 48 - protein, 119 aa, synthetic construct
  REGION 1..119: note = Synthetic Construct
QVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 49 - protein, 119 aa, synthetic construct
  REGION 1..119: note = Synthetic Construct
EVQLVESGAGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 50 - protein, 119 aa, synthetic construct
  REGION 1..119: note = Synthetic Construct
EVQLVESGGGVVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 51 - protein, 119 aa, synthetic construct
  REGION 1..119: note = Synthetic Construct
EVQLVESGGGLKKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 52 - protein, 119 aa, synthetic construct
  REGION 1..119: note = Synthetic Construct
EVQLVESGGGLVKPGSSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 53 - protein, 119 aa, synthetic construct
  REGION 1..119: note = Synthetic Construct
EVQLVESGGGLVKPGGSLRVSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 54 - protein, 119 aa, synthetic construct
  REGION 1..119: note = Synthetic Construct
EVQLVESGGGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 55 - protein, 115 aa, synthetic construct; identical to SEQ ID NO: 12
  REGION 1..115: note = Synthetic Construct

SEQ ID NO: 56 - protein, 447 aa, synthetic construct
  REGION 1..447: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK